(19) United States
(12) Patent Application Publication
(10) Pub. No.: US 2025/0090547 A1
(43) Pub. Date: Mar. 20, 2025

(54) EPA-EE LIPID NANOCOMPOSITE, FORMULATION THEREOF, PREPARATION METHOD THEREFOR, AND APPLICATION THEREOF

(71) Applicant: SHANGHAI INSTITUTE OF MATERIA MEDICA, CHINESE ACADEMY OF SCIENCES, Shanghai (CN)

(72) Inventors: Yong GAN, Shanghai (CN); Xinxin ZHANG, Shanghai (CN); Linmiao GUO, Shanghai (CN); Yunqiu MIAO, Shanghai (CN); Xiaoli WANG, Shanghai (CN); Ying ZHANG, Shanghai (CN); Chunliu ZHU, Shanghai (CN)

(21) Appl. No.: 18/724,128

(22) PCT Filed: Feb. 28, 2023
(86) PCT No.: PCT/CN2023/078639

§ 371 Date: Jun. 25, 2024

(30) Foreign Application Priority Data
Dec. 29, 2021 (CN) 202111640995.4

(51) Int. Cl. A61K 31/557; A61K 9/00; A61K 9/107; A61K 47/24

(52) U.S. Cl. CPC A61K 31/557; A61K 9/0053; A61K 9/1075; A61K 47/24

(57) ABSTRACT

The present application relates to an EPA-EE nano-lipid composition and a formulation, a preparation method, and an application thereof. The EPA-EE nano-lipid composition includes a raw material having a high content of EPA-EE as a primary ingredient and includes an emulsifier containing a highly unsaturated phospholipid. The EPA-EE nano-lipid composition can be prepared into a sub-micron emulsion at nanoscale for use as an oral formulation.

### CROSS-REFERENCE TO RELATED APPLICATIONS

This application claims priority to Chinese Patent Application No. 202111640995.4, filed on Dec. 29, 2021, entitled “EPA-EE NANO-LIPID COMPOSITION, FORMULATION THEREOF, PREPARATION METHOD THEREOF, AND APPLICATION THEREOF”, the content of which is hereby incorporated by reference in its entirety.

### TECHNICAL FIELD

The present application relates to the technical field of oral formulations, particularly relates to an EPA-EE nano-lipid composition, a formulation thereof, a preparation method thereof, and an application thereof.

### BACKGROUND

With the acceleration of life pace, high-sugar and high-fat foods have become popular choices for quick meals and stress relief snacks. On the basis of an imbalanced nutritional structure, due to the factors such as smoking, lack of exercise, and obesity, the incidence of dyslipidemia in China has been steadily increasing each year, leading to a noticeable rise in cardiovascular and cerebrovascular diseases. Relevant surveys indicate significantly elevated levels of total cholesterol (TC), triglycerides (TG), and low-density lipoprotein cholesterol (LDL-C) among residents in China.

Blood viscosity increases in individuals with hyperlipidemia or severe hypertriglyceridemia, resulting in slowed arterial blood flow and increased risk of atherosclerosis. The atherosclerosis is mainly manifested as the deposition of fat and calcium substances on arterial walls, leading to inadequate cardiovascular and cerebrovascular perfusion. In addition, occlusive atherosclerosis is often accompanied with clinical symptoms such as ulcers, pain, and cold sensations. Apolipoproteins are the protein components of plasma lipoproteins, which can bind and transport blood lipids to various tissues of the body for metabolism and utilization, playing a crucial role in development of atherosclerosis. For instance, high-density lipoprotein (HDL) can transport cholesterol deposited in blood vessels, which can then be eliminated from the body through the liver, thereby playing a positive role in mitigating atherosclerosis progression. Conversely, other lipoproteins such as very low-density lipoprotein (VLDL) and low-density lipoprotein (LDL) promote deposition of carried lipids in blood vessels, accelerating plaque formation and potentially leading to cardiovascular diseases such as arterial plaques, myocardial infarction, ischemic heart disease, angina pectoris, and stroke.

Currently, there are no medications available in clinical practice that can directly treat atherosclerosis. Typically, treatments focus on managing diseases associated with its development to prevent plaque deterioration. For instance, statins are currently commonly used lipid-lowering medications in clinical practice, which restrict the liver-based cholesterol synthesis pathway to lower blood lipids and stabilize plaques. Anti-inflammatory medications, anticoagulants, and antihypertensive medications are also utilized in treatment of advanced atherosclerosis. However, these treatments only alleviate disease progression. In addition, prolonged or high-dose usage of various medications can lead to significant toxic side effects on organs such as the liver and kidneys, as well as increase the risk of bleeding. Therefore, developing therapeutic strategies that can safely and effectively stabilize plaques and reverse plaque formation remains a significant clinical challenge in the prevention and treatment of arterial plaques.

Researches have shown that omega-3 polyunsaturated fatty acids (omega-3 PUFAs), primarily including eicosapentaenoic acid (EPA), docosahexaenoic acid (DHA), and α-linolenic acid (ALA), have lipid-regulating effects and can improve health of the circulatory system. Omega-3 PUFAs, derived from deep-sea fish oils, are poorly water-soluble. Common Omega-3 PUFA products on the market are predominantly in the form of soft capsules, such as fish oil soft capsules and eicosapentaenoic acid soft capsules. The patent document CN104856985A provides a composition for EPA ethyl ester (EE) capsules (Vascepa) using high-purity EE type EPA. However, soft capsule formulations have poor absorption rates, especially in fasting conditions, with a bioavailability of less than 20%, hindering efficient absorption of EPA and effective treatment of atherosclerosis. Reported EPA formulations have low purities and poor absorption rates, resulting in low concentrations in blood and rapid metabolism, which cannot maintain necessary concentrations in blood for therapeutic efficacy, leading to suboptimal lipid-lowering and atherosclerosis treatment effects. Therefore, there is a need to develop an EPA formulation that enhances bioavailability while prolonging the duration of effective drug concentration in blood, thereby providing a potential therapeutic medicine for cardiovascular diseases, especially atherosclerosis.

### SUMMARY

In view of this, an objective of the present application is to provide an EPA-EE nano-lipid composition having blood lipid-lowering and arterial plaque-reducing effects, which can be used as an oral formulation and applied in prevention and/or treatment of cardiovascular diseases.

The above objective can be achieved by the following technical solutions.

According to a first aspect of the present application, an eicosapentaenoic acid ethyl ester (EPA-EE) nano-lipid composition is provided. The EPA-EE nano-lipid composition includes the following components in parts by mass: 1 to 30 parts of an EPA-EE raw material, 0.1 to 10 parts of a first emulsifier, 0 to 10 parts of a second emulsifier, 0 to 5 parts of a stabilizer, 0 to 5 parts of a first auxiliary material, and 0 to 15 parts of a second auxiliary material.

In the EPA-EE nano-lipid composition:
the mass content of EPA-EE in the EPA-EE raw material is greater than or equal to 60%;
the first emulsifier is a highly unsaturated phospholipid with an iodine value greater than or equal to 70;
the mass proportion of phosphatidylcholine in the highly unsaturated phospholipid is greater than or equal to 50%;
the composition of the second emulsifier is different from that of the first emulsifier, and the second emulsifier is one or more selected from food-acceptable or pharmaceutically acceptable raw or auxiliary materials;
the stabilizer is a non-ionic polymer;
the first auxiliary material is an auxiliary material that promotes EPA to bind with a lipoprotein;
the second auxiliary material is one or more selected from food-acceptable or pharmaceutically acceptable raw or auxiliary materials and is different from any one of the first emulsifier, the second emulsifier, the stabilizer, and the first auxiliary material.

In some embodiments, the EPA-EE nano-lipid composition further includes water.

In some embodiments, the EPA-EE nano-lipid composition includes the following components in weight percentages based on the total weight of the EPA-EE nano-lipid composition:
the EPA-EE raw material 1% to 30% (w/w);
the first emulsifier 0.1% to 10% (w/w);
the second emulsifier 0% to 10% (w/w);
the stabilizer 0 to 5% (w/w);
the first auxiliary material 0 to 5% (w/w);
the second auxiliary material 0 to 15% (w/w); and
water;
   wherein the mass content of EPA-EE in the EPA-EE raw material is greater than or equal to 60%;
   the first emulsifier is a highly unsaturated phospholipid with an iodine value greater than or equal to 70;
   the mass proportion of phosphatidylcholine in the highly unsaturated phospholipid is greater than or equal to 50%;
   the composition of the second emulsifier is different from that of the first emulsifier, and the second emulsifier is one or more selected from food-acceptable or pharmaceutically acceptable raw or auxiliary materials;
   the stabilizer is a non-ionic polymer;
   the first auxiliary material is an auxiliary material that promotes EPA to bind with a lipoprotein; and
   the second auxiliary material is one or more selected from food-acceptable or pharmaceutically acceptable raw or auxiliary materials and is different from any one of the first emulsifier, the second emulsifier, the stabilizer, and the first auxiliary material;
   the weight percentage of water in the EPA-EE nano-lipid composition is at least 65% (w/w).

The total weight of the EPA-EE nano-lipid composition is 100%, meaning that a sum of the weight percentages of the EPA-EE raw material, the first emulsifier, the second emulsifier, the stabilizer, the first auxiliary material, the second auxiliary material, and water is not more than 100%, and in some embodiments, is 100%.

In some embodiments of the present application, at least one feature from groups (i) and (ii), at least one feature from group (i), or at least one feature from group (ii) is satisfied:
(i) the content of the stabilizer is in a range from 0.1% to 5% (w/w) based on the total weight of the EPA-EE nano-lipid composition;
(ii) the content of the first emulsifier is in a range from 0.01% to 5% (w/w) based on the total weight of the EPA-EE nano-lipid composition.

In some embodiments, the EPA-EE nano-lipid composition includes the following components in weight percentages based on the total weight of the EPA-EE nano-lipid composition:
the EPA-EE raw material 4% to 20% (w/w);
the emulsifier 0.5% to 5% (w/w);
the stabilizer 0.1% to 3% (w/w);
the first auxiliary material 0.1% to 3% (w/w);
the second auxiliary material 0.01% to 10% (w/w); and
water.

In some embodiments of the present application, any one or any combination of the following features is satisfied:
the EPA-EE raw material is an ethyl esterification product of one or more oils selected from a deep-sea fish oil, a seaweed oil, a krill oil, etc.;
the mass content of EPA-EE in the EPA-EE raw material is greater than or equal to 70%;
the iodine value of the first emulsifier (the highly unsaturated phospholipid) is greater than or equal to 90;
the mass proportion of phosphatidylcholine in the highly unsaturated phospholipid is greater than or equal to 70%;
the first emulsifier is one or more selected from soybean phospholipid, sunflower seed phospholipid, and polyene phosphatidylcholine;
the stabilizer is an amphiphilic non-ionic polymer, and the stabilizer is one or more selected from a vitamin lipid polymer derivative, a phospholipid polymer derivative, a fatty acid ester polymer derivative, and a polyoxyethylene-polyoxypropylene ether block copolymer;
optionally, the vitamin lipid polymer derivative is vitamin E polyethylene glycol succinate;
optionally, the phospholipid polymer derivative is a polyethylene glycol-modified synthetic phospholipid;
optionally, the fatty acid ester polymer derivative is a polyethylene glycol-modified fatty acid ester;
a molecular weight of a polyethylene glycol (PEG) unit in the phospholipid polymer derivative is in a range from 400 Da to 6000 Da;
a molecular weight of a PEG unit in the fatty acid ester polymer derivative is in a range from 200 Da to 4000 Da;
the first auxiliary material is one or more selected from an amino acid with a side chain having a negatively charged group, an amino acid derivative with a negatively charged group, and a small peptide with a side chain having a negatively charged group;
the second auxiliary material is one or more selected from an antioxidant, a base oil, a co-emulsifier, a flavoring agent, an interfacial membrane stabilizer, a thickener, and a pH adjuster; and

the EPA-EE nano-lipid composition is in form of submicron emulsion with an average particle size less than or equal to 500 nm.

In some embodiments, any one or any combination of the following features is satisfied:
the iodine value of the first emulsifier is greater than 90, and the first emulsifier is one or more selected from soybean phospholipid, sunflower seed phospholipid, and polyene phosphatidylcholine;
the second emulsifier is one or more selected from a phospholipid different from the first emulsifier, sucrose ester, citric and fatty acid esters of glycerol, fatty acid glyceride, glyceryl monolinoleate, glycerol monostearate, polysorbate, sorbitan fatty acid ester, polyoxyethylene fatty acid ester, Span, alginate, sodium oleate, and caseinate;
the stabilizer includes a terminal group provided by a PEG unit, and the terminal group is OH or methoxy;
the vitamin lipid polymer derivative is one or more selected from d-α-tocopheryl polyethylene glycol 200 succinate, d-α-tocopheryl polyethylene glycol 400 succinate, d-α-tocopheryl polyethylene glycol 1000 succinate, d-α-tocopheryl polyethylene glycol 1500 succinate, d-α-tocopheryl polyethylene glycol 2000 succinate, and d-α-tocopheryl polyethylene glycol 4000 succinate;
the phospholipid polymer derivative is selected from distearoylphosphatidylethanolamine-polyethylene glycol 2000, distearoylphosphatidylethanolamine-polyethylene glycol 5000, dipalmitoylphosphatidylethanolamine-methoxypolyethylene glycol 2000, dipalmitoylphosphatidylethanolamine-methoxypolyethylene glycol 5000, soybean phosphatidylethanolamine-polyethylene glycol monomethyl ether 2000, 1,2-di-myristoyl-rac-glycero-3-methoxypolyethylene glycol 2000, dilauroylphosphatidylethanolamine-polyethylene glycol 2000, and dioleoylphosphatidylethanolamine-polyethylene glycol;
the fatty acid ester polymer derivative is one or more selected from polyethylene glycol 400 oleate, polyethylene glycol 600 oleate, polyethylene glycol 4000 oleate, polyethylene glycol 6000 oleate, polyethylene glycol 400 dioleate, polyethylene glycol 600 dioleate, polyethylene glycol 200 laurate, polyethylene glycol 200 dilaurate, polyethylene glycol 400 laurate, polyethylene glycol 400 dilaurate, polyethylene glycol 400 stearate, and polyethylene glycol 400 distearate;
the polyoxyethylene-polyoxypropylene ether block copolymer is one or more selected from Pluronic L65 and Pluronic F68;
in the first auxiliary material, the amino acid with the side chain having the negatively charged group is one or more selected from aspartic acid, glutamic acid, and taurine;
in the first auxiliary material, the amino acid derivative with the side chain having the negatively charged group is one or more selected from phosphatidylserine, dihexadecyl-glutamate-glutamine, dihexadecyl-glutamate-glutamic acid, dihexadecyl-glutamate-asparagine;
in the first auxiliary material, the small peptide with the side chain having the negatively charged group is glutathione;
in the second auxiliary material, the antioxidant is one or more selected from vitamin E, α-tocopherol, β-tocopherol, γ-tocopherol, mixed tocopherols, α-tocopheryl acetate, β-tocopheryl acetate, γ-tocopheryl acetate, mixed tocopheryl acetates, ascorbic acid, ascorbyl palmitate, ascorbyl stearate, ascorbyl myristate, sodium ascorbate, butylated hydroxyanisole, dibutylated hydroxytoluene, propylgallate, tert-butylhydroquinone, etc.;
in the second auxiliary material, the base oil is one or more selected from soybean oil, olive oil, jojoba oil, sweet almond oil, grapeseed oil, corn oil, walnut oil, sea buckthorn oil, olive oil, coix seed oil, grapeseed oil, ginger oil, coconut oil, camellia oil, rose oil, peppermint oil, lemon oil, medium-chain triglycerides, etc.; and
optionally, the EPA-EE nano-lipid composition is in form of submicron emulsion with an average droplet diameter in a range from 100 nm to 300 nm.

In some embodiments, the EPA-EE nano-lipid composition satisfies any one or any combination of the following features:
the first emulsifier is one or more selected from soybean phospholipid S75, soybean phospholipid S100, sunflower seed phospholipid H100, and polyene phosphatidylcholine;
the second emulsifier is one or more selected from egg yolk lecithin E80, polysorbate 80, glyceryl monolinoleate, and sorbitan oleate 80 (optionally, the second emulsifier is one or more selected from egg yolk lecithin E80, polysorbate 80, and sorbitan oleate 80);
the stabilizer is one or more selected from TPGS, DSPE-PEG, and S40; optionally, the stabilizer is a combination of TPGS and S40, and further optionally, a mixture of TPGS and S40 in equal masses;
the first auxiliary material is one or more selected from phosphatidylserine, sodium glutamate, and taurine; optionally, the first auxiliary material is a combination of taurine and sodium glutamate, and further optionally, a mixture of taurine and sodium glutamate in equal masses;
the second auxiliary material includes the antioxidant and the base oil; optionally, the second auxiliary material is a combination of the antioxidant and the base oil; further optionally, the antioxidant is α-tocopherol, and the base oil is corn oil, olive oil, a medium-chain triglyceride, or a combination thereof; further optionally, the base oil is a combination of corn oil and olive oil; furthermore optionally, the base oil is a mixture of corn oil and olive oil in equal masses.

In some embodiments, the EPA-EE nano-lipid composition includes the following components in parts by mass: 50 to 500 parts by mass of the EPA-EE raw material, 10 to 100 parts by mass of the first emulsifier, 0 to 100 parts by mass of the second emulsifier, 0 to 1.2 parts by mass of the α-tocopherol, 0 to 60 parts by mass of the base oil, and water.

In some embodiments, the EPA-EE nano-lipid composition includes the following components in parts by mass: 50 to 500 parts by mass of the EPA-EE raw material, 10 to 100 parts by mass of the first emulsifier, 0 to 60 parts by mass of the stabilizer, 0 to 50 parts by mass of the first auxiliary material, and water.

According to a second aspect of the present application, an EPA-EE nano-lipid formulation is provided, which includes the EPA-EE nano-lipid composition in the first aspect of the present application. Further, the EPA-EE nano-lipid formulation is an oral formulation.

According to a third aspect of the present application, a preparation method of an EPA-EE nano-lipid formulation is provided. The preparation method can be used to prepare the EPA-EE nano-lipid formulation in the second aspect of the present application.

In some embodiments, the preparation method includes the following steps:
mixing oil phase components including the EPA-EE raw material under a heating condition to prepare an oil phase matrix;
dissolving aqueous phase components into an aqueous solvent to prepare an aqueous phase matrix, or adopting water as the aqueous phase matrix;
mixing and shear-stirring the oil phase matrix and the aqueous phase matrix to prepare an oil-in-water primary emulsion;
subjecting the oil-in-water primary emulsion to high pressure homogenization to prepare a submicron emulsion;
optionally, subjecting the prepared submicron emulsion to filtration, encapsulation, or sterilization.

According to a fourth aspect of the present application, an application of the EPA-EE nano-lipid composition in the first aspect of the present application, the EPA-EE nano-lipid formulation in the second aspect of the present application, or the EPA-EE nano-lipid formulation prepared by the preparation method in the third aspect of the present application is provided. Further, the application includes an application in preparation of a drug for preventing and/or treating a cardiovascular disease or an application in medical foods or health foods.

In some embodiments of the present application, the cardiovascular disease is atherosclerosis.

According to a fifth aspect of the present application, a method for preventing or treating a cardiovascular disease is provided, including administering to a subject a therapeutically effective amount of the EPA-EE nano-lipid composition in the first aspect of the present application, or administering to a subject a therapeutically effective amount of the EPA-EE nano-lipid formulation in the second aspect of the present application, or administering to a subject a therapeutically effective amount of the EPA-EE nano-lipid formulation prepared by the preparation method according to the third aspect of the present application.

The EPA-EE nano-lipid composition provided in the present application, with eicosapentaenoic acid ethyl ester (EPA-EE) as the primary ingredient, includes EPA-EE, an emulsifier, and water, wherein the content of EPA-EE in the EPA-EE raw material is relatively high (e.g., with a mass percentage ≥60%), and thus the proportion of inactive fatty acids is reduced. The composition can be prepared into a submicron emulsion at nanoscale (optionally with an average particle size ≤500 nm), which, as an oral formulation, can maintain the effective blood eicosapentaenoic acid (EPA) concentration for a long period and enhance the oral absorption and bioavailability of EPA. The emulsifier used in the EPA-EE nano-lipid composition includes the highly unsaturated phospholipid (referred to as the first emulsifier, optionally with an iodine value ≥70), enabling improved treatment effects on arterial plaques. The stabilizer introduced into the EPA-EE nano-lipid composition can help in maintaining high blood EPA concentration and enhancing the pharmaceutical efficacy. The lipoprotein binding promoter (referred to as the first auxiliary material) introduced into the EPA-EE nano-lipid composition can promote EPA to bind with the lipoprotein and increase the content of EPA in the lipoprotein, resulting in blood lipid-lowering and atherosclerotic plaque-reducing effects and promoting the application in prevention and/or treatment of cardiovascular diseases, particularly in prevention and/or treatment of atherosclerosis. The nano-lipid formulation for oral administration prepared from the EPA-EE nano-lipid composition with both the stabilizer and the first auxiliary material (i.e., the lipoprotein binding promoter) added can achieve a synergistic effect, which increases the blood EPA concentration and maintains the high blood EPA concentration for a long period, while promoting the binding of the lipoprotein with EPA, increasing the content of EPA in the lipoprotein, accelerating in vivo saturated fatty acid metabolism, enhancing blood lipid-lowering and atherosclerotic plaque-reducing effects. This is significant for efficient blood lipid-lowering and atherosclerosis prevention and/or treatment. The EPA-EE as the active ingredient, the first emulsifier, the stabilizer, the first auxiliary material, and other components in the composition work together to enhance the treatment effect on arterial plaques.

The EPA-EE nano-lipid composition and the formulation (optionally oral formulation) thereof provided in the present application can be applied to the fields such as medical foods, health foods, and pharmaceuticals.

### BRIEF DESCRIPTION OF THE DRAWINGS

In order to illustrate the technical solutions in the embodiments of the present application more clearly, and to understand the present application and its beneficial effects more completely, the accompanying drawings, which are to be used in the description of the embodiments, will be briefly described below.

FIG. 1 shows a histogram of EPA contents in low-density lipoprotein in bloods of rats after continuous 8 weeks of oral administration of different formulation groups, with a dosage of EPA of 400 mg/kg.

FIG. 2 shows a histogram of ratios of plaque area to vessel area in APOE⁻/− mice after continuous 8 weeks of oral administration of different formulation groups.

In the figures, n.s. means P>0.05, “**” means P<0.01, and “***” means P<0.001.

### DETAILED DESCRIPTION

The present application will be described in further detail below in conjunction with the accompany drawings, the embodiments, and the examples. It should be understood that these embodiments and examples are only for illustrating the present application and not intended to limit the scope of the present application. The purpose of providing these embodiments and examples is to provide a more thorough and comprehensive understanding of the disclosed content of the present application. It should also be understood that the present application may be implemented in many different forms and is not limited to the embodiments and examples described herein. Those skilled in the art may make various changes and modifications without departing from the concept of the present application. The obtained equivalent forms also fall within the protection scope of the present application. Furthermore, in the following description, numerous specific details are given in order to provide a full understanding of the present application. However, it should be understood that the present application can be implemented without one or more of these details.

Unless defined otherwise, all technical and scientific terms used herein have the same meaning as commonly understood by one of ordinary skill in the art to which the present application belongs. The terminology used herein in the description of the present application is for the purpose of describing the embodiments and the examples only and is not intended to limit the present application.

### Terms

Unless otherwise stated or there is a contradiction, the terms or phrases used herein have the following meanings.

The terms “and/or”, “or/and”, and “as well as/or” used herein, when used in a list of two or more associated items, mean that any one of the items can be selected, or any or all combinations of the items can be selected. The any or all combinations include a combination of any two of the listed items, a combination of any more of the listed items, or a combination of all of the listed items. It is to be noted that in the present application, when at least three items are connected by at least two conjunction combinations selected from “and/or”, “or/and”, and “as well as/or”, the technical solutions undoubtedly include all technical solutions that are connected by “logical AND” and also undoubtedly includes all technical solutions that are connected by “logical OR”. For example, “A and/or B” includes three parallel solutions, i.e., A, B, and A+B. For another example, “A, and/or B, and/or C, and/or D” includes any one of A, B, C, D (i.e., the technical solutions connected by “logical OR”), and combinations of any or all of A, B, C, D, such as a combination of any two or three of A, B, C, D and a combination of A, B, C, and D (i.e., the technical solutions connected by “logical AND”).

In the present application, when at least three features are connected by at least two conjunction combinations selected from “and/or”, “or/and”, and “as well as/or”, it is equivalent to the expression “including one or more of the features”. For example, “TA, and/or TB, and/or TC, and/or TD” is equivalent to “including one or more of the following features: TA, TB, TC, and TD”.

In the present application, unless otherwise specifically defined, “multiple” and “more” refer to a quantity equal to or greater than 2. For example, “one or more” means one or two or more.

In the present application, unless otherwise specified, “one or more” refers to any one of the listed items or any combination of the listed items. Likewise, other expressions indicating “one or more”, such as “one or multiple”, should be understood in the same manner unless otherwise specified. As used herein, terms such as “a combination thereof”, “any combination thereof”, and “any combination manner thereof” includes all suitable combinations of any two or more of the listed items.

As used herein, “suitable” mentioned in “suitable combination”, “suitable manner” “any suitable manner”, etc., is defined based on the ability to implement the technical solutions of the present application, to solve the technical problems as described in the present application, and to achieve the expected technical effects as described in the present application.

As used in the present application, “having”, “containing”, “comprising”, and “including” are synonymous, which are inclusive or open-ended, not excluding additional or unlisted members, elements, or method steps.

As used herein, “preferred”, “preferably”, “better”, “advisable”, “for example”, “such as”, “as an example”, and “exemplary” are only used to describe more effective or detailed embodiments or examples. They indicate a relationship between the preceding and following technical solutions, but should not be understood as limiting the scope of the present application. In the present application, unless otherwise specified, “A (such as B)” indicates that B is a non-limiting example of A, and it should be understood that A is not limited to B.

In the present application, “further”, “furthermore”, “particularly”, and the like are only for descriptive purposes and indicate differences in content, but should not be construed as limiting the protection scope of the present application.

In the present application, “optionally”, “optional” and “option” mean that the feature being modified is not mandatory, indicating a choice between with or without the feature. If there are multiple “optionally” in a same technical solution, unless otherwise specified or there is a contradiction or mutual restriction, each “optionally” is independent from each other. In the present application, the expressions such as “optionally comprising” and “optionally including” mean “including or not including”; “optional component X” means that the component X may be present or absent.

In the present application, terms such as “first”, “second”, “third”, and “fourth” in “first aspect”, “second aspect”, “third aspect”, “fourth aspect”, “first auxiliary material”, “second auxiliary material”, etc. are only for descriptive purposes and should not be understood as indicating or implying relative importance or quantity, nor as implying the importance or quantity of the indicated technical features. Moreover, “first”, “second”, “third”, “fourth”, etc. are only for the purpose of non-exhaustive enumeration and description, and it should be understood that they do not constitute a closed limitation on the quantity.

In the present application, an open-ended description for the technical features encompasses not only a close-ended technical solution consisting of the features, but also an open-ended technical solution including the features.

In the present application, when a numerical interval (i.e., a numerical range) is mentioned, unless otherwise specified, suitable values are considered as continuously distributed within this numerical interval and include two numerical endpoints (i.e., the minimum value and the maximum value) of the numerical interval, as well as every value between the two numerical endpoints. Unless otherwise specified, when a numerical interval only points to integers within the numerical interval, the numerical interval includes the two endpoint integers of the numerical range and every integer between the two endpoints. In addition, when multiple ranges are provided to describe a characteristic or a feature, these ranges can be combined. In other words, unless otherwise indicated, ranges disclosed herein should be understood to include any or all sub-ranges subsumed therein.

In the present application, when a numerical value or a numerical range is mentioned, unless otherwise specified, it should be understood that the numerical range encompasses reasonable approximations of the two endpoints. A person skilled in the art can understand that the fluctuations brought by these approximations are all included within the interval covered by the indicated numerical range. Therefore, in the present application, unless otherwise specified, “N1” and “about N1” have the same meaning and can be used interchangeably, and “N1 to N2” and “about N1 to about N2” have the same meaning and can be used interchangeably, where N1 and N2 are two different numerical values. Due to one or more factors such as reasonable deviations allowed in the field, instrument precision, and so on, the approximate values within the approximation range should also be considered within the scope of the numerical range. For example, “a temperature of 20° C. to 30° C.” should be understood as “about 20° C. to about 30° C.”. Furthermore, taking an endpoint of “20° C.” with an approximation of 1° C. as an example, the approximate values such as 19° C. and 19.5° C. within the approximation range of “about 20° C.” should also be included in the range of 20° C. to 30° C.

In the present application, when an approximation is involved, unless otherwise specified, the fluctuation range is generally ±10%, but can also be ±8%, ±5%, ±3%, etc. An approximation in the present application provides both the stated numerical value and the numerical range represented by that approximation. For instance, “about 200 nm” provides both the technical solution of “being 200 nm” and the technical solution of the numerical range defined by “200 nm±a fluctuation range”.

In the present application, when a temperature parameter is mentioned, unless otherwise specifically stated, not only a thermostatic process but also a variation within a certain temperature interval is allowed. It should be understood that the thermostatic process allows for temperature fluctuations within the accuracy range of the instrument, such as fluctuations within the range of ±5° C., ±4° C., ±3° C., ±2° C., or +1° C.

In the present application, the term “room temperature” generally refers to 4° C. to 35° C., preferably 20° C.±5° C. In some embodiments of the present application, “room temperature” refers to 20° C. to 30° C.

In the present application, both % (w/w) and wt % refer to a weight percentage.

All documents mentioned in the present application are incorporated by reference in this application as if each document is individually indicated to be incorporated by reference. Unless there is a conflict with the objects and/or technical solutions of the present application, the cited documents involved in the present application are incorporated in their entireties and for all purposes. When a document is cited in this application, the definitions of relevant technical features, terms, nouns, phrases, etc. in the cited document are also cited. When a document is cited in the present application, the examples and preferred modes of the related technical features that are cited can also be incorporated into this application by reference, provided that the present application can be implemented. It should be understood that when the referenced content conflicts with the description in the present application, the present application shall prevail or the referenced content shall be amended adaptively according to the description in the present application.

The masses or weights of the relevant components mentioned in the present application not only refer to the specific contents of the components, but also indicate the proportional relationship between the masses or weights of the components. Therefore, the enlargement or reduction in scale of the contents of the relevant components in the present application is within the scope disclosed by the present application. Specifically, the masses or weights described in the present application may be in units well known in the chemical industry such as μg, mg, g, kg, etc.

In the present application, unless otherwise explicitly stated, the sequence of the steps is not strictly limited, and these steps can be executed in orders other than that in the description. Moreover, any step can include multiple sub-steps or multiple stages. These sub-steps or stages are not necessarily executed at the same time, but can be executed at different times. These sub-steps or stages are not necessarily performed sequentially, but can be performed in turn or alternately or simultaneously with some of other steps or sub-steps or stages of other steps.

Unless otherwise specifically defined, abbreviations used herein have the following meanings: PUFA refers to polyunsaturated fatty acid; omega-3 PUFA refers to omega-3 polyunsaturated fatty acid, EPA refers to eicosapentaenoic acid, DHA refers to docosahexaenoic acid, and PC refers to phosphatidylcholine.

In the present application, the unsaturation degree of a phospholipid is primarily characterized by an iodine value, which, unless otherwise specified, refers to an average iodine value.

As used in the present application, unless otherwise specified, the “highly unsaturated phospholipid” refers to a phospholipid with an iodine value greater than or equal to 70, which can be a phospholipid with a higher iodine value, such as a phospholipid with an iodine value greater than or equal to 90.

In the present application, “submicron emulsion” refers to an emulsion containing droplets with an average particle size ranging from 100 nm to 1000 nm. In some embodiments, the average particle size in any submicron emulsion is independently less than or equal to 500 nm.

In the present application, when a molecular weight is mentioned, unless otherwise specified, the molecular weight refers to either a number-average molecular weight or a weight-average molecular weight, and typically refers to the weight-average molecular weight unless otherwise specified.

In the present application, “small peptide” refers to a peptide with two or three amino acid units and a molecular weight smaller than or equal to 1000 Daltons.

As used in the present application, “medium-chain triglyceride”, also known as “medium-chain triacylglycerol” is abbreviated as MCT, wherein “medium-chain” refers to the moderate length of the fatty acid chain (containing 6, 8, or 12 carbon atoms) thereof. In national food safety standards, the medium-chain triglyceride can be used as a food raw material or an emulsifier.

In the present application, “long-chain moiety” in the stabilizer can preferably refer to a polyethylene glycol segment for a vitamin lipid polymer derivative, a phospholipid polymer derivative, or a fatty acid ester polymer derivative.

In the present application, “polyethylene glycol (PEG)” and “polyoxyethylene (POE)” have the same meaning and can be used interchangeably. For a molecular weight of PEG, unless otherwise specifically defined, it refers to either a number-average molecular weight or a weight-average molecular weight, and typically refers to the weight-average molecular weight unless otherwise specified.

In the present application, unless otherwise specified, “above” and “below” each independently include the stated number.

In the present application, “≥” and “greater than or equal to” have the same meaning and can be used interchangeably, both indicating greater than or equal to. “≤” and “less than or equal to” have the same meaning and can be used interchangeably, both indicating less than or equal to.

### First Aspect of the Present Application

According to a first aspect of the present application, an EPA-EE nano-lipid composition with both improved bioavailability and prolonged duration of effective concentration in blood is provided. The EPA-EE nano-lipid composition, with a raw material having a high content of EPA-EE as the primary ingredient, can be prepared into a submicron emulsion at nanoscale (optionally with an average particle size <500 nm), which, as an oral formulation, can maintain an effective blood EPA concentration for a long period, thereby improving the oral absorption and bioavailability of EPA.

According to some embodiments of the first aspect of the present application, an eicosapentaenoic acid ethyl ester (EPA-EE) nano-lipid composition is provided. The EPA-EE nano-lipid composition includes the following components in parts by mass: 1 to 30 parts of an EPA-EE raw material, 0.1 to 10 parts of a first emulsifier, 0 to 10 parts of a second emulsifier, 0 to 5 parts of a stabilizer, 0 to 5 parts of a first auxiliary material, and 0 to 15 parts of a second auxiliary material.

Further,
the mass content of EPA-EE in the EPA-EE raw material is greater than or equal to 60%;
the first emulsifier is a highly unsaturated phospholipid with an iodine value greater than or equal to 70;
the mass proportion of phosphatidylcholine in the highly unsaturated phospholipid is greater than or equal to 50%;
the composition of the second emulsifier is different from that of the first emulsifier, and the second emulsifier is one or more selected from food-acceptable or pharmaceutically acceptable raw or auxiliary materials;
the stabilizer is a non-ionic polymer;
the first auxiliary material is an auxiliary material that promotes EPA to bind with a lipoprotein;
the second auxiliary material is one or more selected from food-acceptable or pharmaceutically acceptable raw or auxiliary materials and is different from any one of the first emulsifier, the second emulsifier, the stabilizer, and the first auxiliary material;
further, the total weight of the EPA-EE nano-lipid composition can be 900 to 1100 parts by mass, optionally 1000 parts by mass.

In the present application, unless otherwise stated, a specific weight expressed by “1 part by mass” is not specifically limited and can be any suitable value such as 1 g, 0.5 g, 1 mg, 0.5 mg, 1 μg, etc.

In some embodiments, the EPA-EE nano-lipid composition further includes water;
optionally, the amount of water in the EPA-EE nano-lipid composition is greater than or equal to 65 parts by mass; further optionally, the amount of water in the EPA-EE nano-lipid composition is 65 to 89.8 parts by mass; yet further optionally, the amount of water in the EPA-EE nano-lipid composition is 65 to 80 parts by mass; yet further optionally, the amount of water in the EPA-EE nano-lipid composition is 65 to 75 parts by mass;
optionally, the total amount of the EPA-EE nano-lipid composition is 100 parts by mass.

According to some embodiments of the first aspect of the present application, the EPA-EE nano-lipid composition includes the following components in weight percentages based on the total weight of the EPA-EE nano-lipid composition:
the EPA-EE raw material 1% to 30% (w/w);
the first emulsifier 0.1% to 10% (w/w);
the second emulsifier 0% to 10% (w/w);
the stabilizer 0 to 5% (w/w);
the first auxiliary material 0 to 5% (w/w);
the second auxiliary material 0 to 15% (w/w); and
water.

Further, the mass content of EPA-EE in the EPA-EE raw material is greater than or equal to 60%;
the first emulsifier is a highly unsaturated phospholipid with an iodine value greater than or equal to 70;
the mass proportion of phosphatidylcholine in the highly unsaturated phospholipid is greater than or equal to 50%;
the composition of the second emulsifier is different from that of the first emulsifier, and the second emulsifier is one or more selected from food-acceptable or pharmaceutically acceptable raw or auxiliary materials; the stabilizer is a non-ionic polymer for stabilizing the emulsion and effectively prolonging the blood EPA concentration;
the first auxiliary material is an auxiliary material that promotes EPA to bind with a lipoprotein;
the second auxiliary material is one or more selected from food-acceptable or pharmaceutically acceptable raw or auxiliary materials, and is different from any one of the first emulsifier, the second emulsifier, the stabilizer, and the first auxiliary material.

It should be understood that water is present in the EPA-EE nano-lipid composition in a suitable amount. In some embodiments, the weight percentage of water in the EPA-EE nano-lipid composition is at least 65% (w/w), and the total weight percentage of the above components in the EPA-EE nano-lipid composition is not more than 100%.

In some embodiments, the total weight percentage of the EPA-EE raw material, the first emulsifier, the second emulsifier, the stabilizer, the first auxiliary material, the second auxiliary material, and water is not more than 100%, optionally, is 100%.

In some embodiments of the present application, the EPA-EE nano-lipid composition includes, in weight percentages, 1% to 30% (w/w) of the EPA-EE raw material, 0.1% to 10% (w/w) of the highly unsaturated phospholipid (referred to as the first emulsifier), 0% to 10% (w/w) of an additional emulsifier (referred to as the second emulsifier), 0% to 10% of an auxiliary material that promotes EPA to bind with a lipoprotein (referred to as the first auxiliary material), 0% to 40% of an additional food and/or pharmaceutically acceptable raw or auxiliary material (referred to as the second auxiliary material), and water in a suitable amount. The EPA-EE raw material provides a high content of active EPA-EE, the first emulsifier provides the highly unsaturated phospholipid with the high iodine value, the stabilizer is adopted to enhance the stability of the nano-lipid composition system, and the first auxiliary material provides the lipoprotein binding promoter for EPA. The various components work collectively together to improve the oral absorption and bioavailability of EPA. The second auxiliary material can be one or more selected from additional food-acceptable raw or auxiliary materials and additional pharmaceutically acceptable raw or auxiliary materials.

The EPA-EE nano-lipid composition provided in the present application contains EPA-EE, the emulsifier, and water, wherein the high content of EPA-EE is provided by the EPA-EE raw material (e.g., the mass percentage of EPA-EE in the EPA-EE raw material is greater than or equal to 60%). The EPA-EE nano-lipid composition can be prepared into a submicron emulsion at nanoscale (optionally with an average particle size <500 nm), which, when used as an oral formulation, can maintain the effective EPA concentration in blood for a long period, thereby enhancing the oral absorption and bioavailability of EPA.

Through extensive explorations and researches, the inventors have found that EPA is an active fatty acid playing a crucial role in treating cardiovascular diseases. The binding between EPA and low-density lipoprotein can prevent the formation of oxidized low-density lipoprotein, which is crucial for EPA to exhibit its efficacy in lowering blood lipids. In addition, the EPA formulation containing EPA as the active ingredient need to provide a high EPA concentration (or exposure level) in blood and maintain a relatively long exposure period to achieve effective binding between EPA and low-density lipoprotein. The significant reduction in oxidized low-density lipoprotein is conducive to alleviating inflammatory reaction and endothelial cell damage at atherosclerotic sites, thereby promoting the treatment of atherosclerosis.

In the EPA-EE nano-lipid composition as described above, the first emulsifier is adopted to emulsify the formulation. Additionally, the unsaturation degree of the phospholipid can affect the pharmaceutical efficacy. The highly unsaturated phospholipid is more beneficial for atherosclerosis treatment, and the phospholipid with the iodine value above 70 contributes to improvement of cardiovascular health. In some embodiments of the present application, in addition to the first emulsifier (the highly unsaturated phospholipid), an additional emulsifier (referred to as the second emulsifier) can be included in the EPA-EE nano-lipid composition to provide flexibility in controlling emulsification. The content of the second emulsifier can be 0 (indicating the absence of the second emulsifier).

In the EPA-EE nano-lipid composition as described above, the content of the stabilizer can be 0 (indicating the absence of the stabilizer). When the content of the stabilizer is not 0, introducing the stabilizer (also referred to as an EPA stabilizer) into the EPA-EE nano-lipid composition can help in maintaining a high EPA concentration in blood and enhancing the pharmaceutical efficacy.

In the EPA-EE nano-lipid composition as described above, the content of the first auxiliary material (also referred to as the lipoprotein binding promoter) can be 0 (indicating the absence of the first auxiliary material). When the content of the first auxiliary material is not 0, introducing the lipoprotein binding promoter into the EPA-EE nano-lipid composition can promote the binding between EPA and the lipoprotein and increase the content of EPA in the lipoprotein, thereby enhancing the blood lipid-lowering effect and the arterial plaque-reducing effect and facilitating the application of the composition in the prevention and/or treatment of cardiovascular diseases, particularly in the prevention and/or treatment of atherosclerosis.

In the present application, unless otherwise specified, “prevention and/or treatment” includes “prevention”, “treatment”, and “prevention and treatment”, and “prevention and treatment” indicates the abilities of both preventing and treating.

In the EPA-EE nano-lipid composition as described above, when neither the content of the stabilizer nor the content of the first auxiliary material is 0, i.e., when both the stabilizer and the first auxiliary material are included, the nano-lipid formulation for oral administration prepared from the EPA-EE nano-lipid composition can achieve a synergistic effect, which increases the blood EPA concentration and maintains the high blood EPA concentration for a long period, while promoting the binding of the lipoprotein with EPA, increasing the content of EPA in the lipoprotein, accelerating in vivo saturated fatty acid metabolism, enhancing blood lipid-lowering and atherosclerotic plaque-reducing effects. This is significant for efficient blood lipid-lowering and atherosclerosis prevention and/or treatment.

In some embodiments of the present application, the EPA-EE nano-lipid composition includes the stabilizer and/or the first auxiliary material. That is, the EPA-EE nano-lipid composition includes at least one of the stabilizer or the first auxiliary material.

In some embodiments of the present application, the EPA-EE nano-lipid composition includes the EPA-EE raw material, the first emulsifier, water, and the stabilizer, and optionally further includes the second emulsifier, the first auxiliary material, and the second auxiliary material.

In some embodiments of the present application, the EPA-EE nano-lipid composition includes the EPA-EE raw material, the first emulsifier, water, and the first auxiliary material, and optionally further includes the second emulsifier, the stabilizer, and the second auxiliary material.

In some embodiments of the present application, the EPA-EE nano-lipid composition includes the EPA-EE raw material, the first emulsifier, water, the stabilizer, and the first auxiliary material, and optionally further includes the second emulsifier, and the second auxiliary material.

In some embodiments of the present application, the EPA-EE nano-lipid composition includes the EPA-EE raw material, the first emulsifier, water, the stabilizer, the first auxiliary material, and the second auxiliary material, and optionally further includes the second emulsifier.

### EPA-EE Raw Material

In the present application, the EPA-EE nano-lipid composition includes the EPA-EE raw material. In some embodiments of the present application, the content of the EPA-EE raw material in the EPA-EE nano-lipid composition is 1% to 30% by weight, and further can be 4% to 20% by weight, and as non-limiting examples, can be any one of the following percentages or in a percentage range formed between any two of the following percentages: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, etc., such as 15% to 30%.

The EPA-EE raw material of the present application can provide a high content of EPA. In the present application, a mass proportion (i.e., purity) of EPA-EE in the EPA-EE raw material can be greater than or equal to 60%, and further can be greater than or equal to 70%. If the content of EPA-EE in the EPA-EE raw material is relatively low (e.g. <40%), after administration, the required exposure level of the active substance for the treatment cannot be achieved. The composition of the present application can encapsulate a high concentration of EPA-EE, so that the formulated composition can meet the required dose of EPA.

In some embodiments of the present application, the EPA-EE raw material is derived from one or more oils selected from a deep-sea fish oil, a seaweed oil, a krill oil, etc.

In some embodiments of the present application, the EPA-EE raw material is an ethyl esterification product of one or more oils selected from a deep-sea fish oil, a seaweed oil, a krill oil, etc. Taken the EPA-EE raw material derived from a deep-sea fish oil as an example, in some embodiments of the present application, the EPA-EE raw material is obtained by ethyl esterification (EE treatment) of the deep-sea fish oil. EPA in fish oil is mainly present in the form of triglyceride. The deep-sea fish oil can be subjected to concentration, hydrolysis, and separation to obtain EPA, which can be then subjected to pre-esterification by reacting EPA with ethanol and concentrated sulfuric acid, followed by a transesterification reaction, which converts eicosapentaenoic acid glyceride into EE form. Through separation, a raw material containing eicosapentaenoic acid ethyl ester (EPA-EE) can be obtained, which is the EPA-EE raw material of the present application. In the obtained raw material, the purity of EPA-EE can be greater than or equal to 60% by mass, and optionally can be greater than 60% by mass. In some preferred embodiments, the purity of EPA-EE in the obtained raw material is greater than or equal to 70%, and further can be greater than 70%.

In some embodiments, the mass proportion of EPA-EE in the EPA-EE raw material is any one of the following percentages or in a percentage range formed between any two of the following percentages: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 95%, 97%, etc.

In some embodiments of the present application, the EPA-EE raw material is selected from any one or any combination of the following products purchased from KinOmega Inc.: 6015 EE EPA60%+DHA12%, KinOmega 7010 EE EPA70%+DHA8%, K85EE Omega-3-acid-EE (EPA EE 86227-47-6), Maxomega EPA 97 EE, etc.

### Emulsifier

In the present application, the EPA-EE nano-lipid composition includes an emulsifier that can form the composition into an emulsion formulation, such as an oral emulsion formulation. The EPA-EE nano-lipid composition of the present application in a unique nano-lipid formulation is capable of encapsulating a high concentration of EPA-EE, so as to meet the dosage requirement of EPA for effective blood lipid-lowering and atherosclerosis treatment in vivo upon oral administration.

In some embodiments of the present application, the content of the emulsifier in the EPA-EE nano-lipid composition is 0.10% to 20% by weight, further can be 0.10% to 10% by weight, yet further can be 0.5% to 5% by weight, and as non-limiting examples, can be any one of the following percentages or in a percentage range formed between any two of the following percentages: 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 2%, etc., the percentage ranges can be such as 0.5% to 10%, 1% to 20%, 1% to 12%, 2% to 12%, etc.

### Highly Unsaturated Phospholipid (First Emulsifier)

The EPA-EE nano-lipid composition of the present application includes a highly unsaturated phospholipid, also referred to as a first emulsifier, which is a phospholipid with an iodine value greater than or equal to 70. In some embodiments, the first emulsifier can be a mixture of two or more phospholipid components, wherein the iodine value of each phospholipid component is greater than or equal to 70, further can be greater than or equal to 80, yet further can be greater than or equal to 90, yet further can be greater than or equal to 100. In some embodiments of the present application, the iodine value of the highly unsaturated phospholipid can be any one of 70, 71, 72, 74, 75, 76, 77, 78, 79, 80, 81, 82, 83, 84, 85, 86, 87, 88, 89, 90, 92, 94, 95, 96, 97, 98, 99, 100, 101, 102, 103, 105, 106, 108, 110, 112, 113, etc., or a range formed between any two of the above mentioned iodine values, for example 85 to 113, 85 to 110, 85 to 105, 85 to 102, 85 to 100, 85 to 95, 90 to 113, 90 to 110, 90 to 105, 90 to 102, 90 to 100, 94 to 97, 95 to 113, 95 to 110, 95 to 105, etc. Any individual iodine value or any iodine value at an endpoint of any range as mentioned above may encompass any suitable fluctuation range such as +1, +2, etc. When the iodine value is greater than 80, an even better treatment effect on arterial plaques can be achieved.

In some embodiments of the present application, the first emulsifier (the highly unsaturated phospholipid) is one or more selected from soybean phospholipid, sunflower seed phospholipid, polyene phosphatidylcholine, etc.

In some embodiments of the present application, the mass proportion of phosphatidylcholine in the phospholipid (in the highly unsaturated phospholipid) of the first emulsifier is greater than or equal to 50%, further can be greater than or equal to 60%, yet further can be greater than or equal to 70%.

In some non-limiting embodiments, the mass content of the phosphatidylcholine in the first emulsifier (the highly unsaturated phospholipid) is, for example, any one of the following percentages or in a percentage range formed between any two of the following percentages: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 85%, 90%, 95%, 98%, etc.

The inventors have also found that the unsaturation degree of the phosphatidylcholine also affects the pharmaceutical efficacy. The phosphatidylcholine with a relatively high unsaturation degree is better for the atherosclerosis treatment. The unsaturation degree of the phosphatidylcholine can likewise be characterized by an iodine value. A higher iodine value indicates a higher unsaturation degree. When the iodine value is greater than 80, a better treatment of atherosclerotic plaques can be achieved. In some embodiments, the iodine value of the phosphatidylcholine is greater than or equal to 80, further can be greater than or equal to 90, yet further can be greater than or equal to 100. In some embodiments of the present application, the iodine value of the phosphatidylcholine is any one of 80, 81, 82, 83, 84, 85, 86, 87, 88, 89, 90, 92, 94, 95, 96, 97, 98, 99, 100, 101, 102, 103, 105, 106, 108, 110, 112, 113, etc., or in a range formed between any two of the above-mentioned iodine values, for example 85 to 113, 85 to 110, 85 to 105, 85 to 102, 85 to 100, 85 to 95, 90 to 113, 90 to 110, 90 to 105, 90 to 102, 90 to 100, 94 to 97, 95 to 113, 95 to 110, 95 to 105, etc. Any individual iodine value or any iodine value at an endpoint of any range as mentioned above may encompass any suitable fluctuation range such as +1, +2, etc. In some embodiments of the present application, the content of the phosphatidylcholine (PC) in the phospholipid is greater than or equal to 50%, and the iodine value of the phosphatidylcholine is greater than 80, which can achieve a better treatment effect on arterial plaques. In some embodiments of the present application, the phosphatidylcholine is one or more selected from soybean phospholipid S75, soybean phospholipid S100, sunflower seed phospholipid H100, and polyene phosphatidylcholine.

In some embodiments, the mass proportion of the phosphatidylcholine in the highly unsaturated phospholipid is greater than or equal to 50%, and the iodine value of the phosphatidylcholine is greater than or equal to 80.

In some embodiments, the iodine value of the first emulsifier (the highly unsaturated phospholipid) is greater than or equal to 80 (which may further be), and the mass proportion of the phosphatidylcholine (PC) in the highly unsaturated phospholipid is greater than or equal to 50%, which can achieve a better treatment effect on atherosclerotic plaques.

In some embodiments, the iodine value of the first emulsifier is greater than 90, and the first emulsifier is one or more selected from soybean phospholipid, sunflower seed phospholipid, and polyene phosphatidylcholine.

Any one of the phospholipid components in the present application can be an individual phospholipid molecule, or a derivative of a phospholipid molecule, or a modified phospholipid.

The phospholipid component in the EPA-EE nano-lipid composition is not limited to being provided by the first emulsifier, but may also be provided by the second emulsifier. However, the phospholipid component provided by the second emulsifier is not the phospholipid with the iodine value greater than or equal to 70. The phospholipid component in the second emulsifier can also be the modified phospholipid as described above.

The first emulsifier can also simultaneously perform other functions in the EPA-EE nano-lipid composition, e.g., the first emulsifier can also simultaneously serve as the first auxiliary material. For example, the first emulsifier can be a liver-targeting molecule modified with the highly unsaturated phospholipid, or the first emulsifier can be the highly unsaturated phospholipid modified with PEG.

In some embodiments, the mass proportion of the phospholipid components each with the iodine value greater than or equal to 70 in all phospholipid components of the EPA-EE nano-lipid composition is greater than 90%.

In some embodiments, the mass proportion of the phospholipid components each with the iodine value greater than or equal to 90 in all phospholipid components of the EPA-EE nano-lipid composition is greater than 90%.

In some preferred embodiments of the present application, the first emulsifier is one or more selected from soybean phospholipid S75, soybean phospholipid S100, sunflower seed phospholipid H100, and polyene phosphatidylcholine.

In some embodiments of the present application, the content of the first emulsifier in the EPA-EE nano lipid composition is 1% to 10% by mass, further can be 5% to 10% by mass, and as non-limiting examples, can be any one of the following percentages or in a percentage range formed between any two of the following percentages: 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, 3.5%, 4%, 4.2%, 4.5%, 4.6%, 4.8%, 5%, 5.5%, 6%, 6.5%, 7%, 7.5%, 8%, 8.5%, 9%, 9.5%, 10%, etc., the percentage ranges can be such as 1% to 5%, 5% to 10%, etc.

The type and amount features of the first emulsifier can be combined in any suitable manner, including, but not limited to, the combinations specifically enumerated herein, which includes, but is not limited to, the combinations set forth in the embodiments given below.

### Second Emulsifier

The second emulsifier in the present application does not include a same component as that in the first emulsifier. That is, the composition of the second emulsifier is different from the composition of the first emulsifier.

In some embodiments of the present application, the emulsifier components in the EPA-EE nano-lipid composition can include an additional emulsifier (also referred to as the second emulsifier) other than the first emulsifier to flexibly control the emulsification. In some embodiments of the present application, the second emulsifier is one or more selected from other phospholipids, such as egg yolk phospholipids, which are different from that the phospholipid in the first emulsifier, sucrose ester, citric and fatty acid esters of glycerol, fatty acid glyceride, glyceryl monolinoleate, glycerol monostearate, polysorbate, sorbitan fatty acid ester, polyoxyethylene fatty acid ester, Span, alginate, sodium oleate, and caseinate. In some embodiments of the present application, the second emulsifier is one or more selected from other phospholipids, such as egg yolk phospholipids, which are different from that in the first emulsifier, sucrose ester, citric and fatty acid esters of glycerol, fatty acid glyceride, polysorbate, sorbitan fatty acid ester, polyoxyethylene fatty acid ester, Span, alginate, and caseinate.

In some embodiments of the present application, the content of the second emulsifier in the EPA-EE nano lipid composition is 0% to 10% by weight, further can be 0% to 5% by weight, and as non-limiting examples, can be any one of the following percentages or in a percentage range formed between any two of the following percentages: 0.01%, 0.02%, 0.03%, 0.04%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, 3.5%, 4%, 4.2%, 4.5%, 4.6%, 4.8%, 5%, 5.5%, 6%, 6.5%, 7%, 7.5%, 8%, 8.5%, 9%, 9.5%, 10%, etc., the percentage ranges can be such as 1% to 2%, 2% to 10%, 1% to 5%, 5% to 10%, etc.

In some embodiments, the second emulsifier is an emulsifier that includes no highly unsaturated phospholipid.

In some embodiments, the second emulsifier includes a phospholipid component that is different from those in the first emulsifier, i.e., not the highly unsaturated phospholipid.

In some embodiments, the second emulsifier includes phospholipid components all of which are saturated phospholipid.

In some embodiments, the second emulsifier includes no phosphatidylcholine component.

In some embodiments, the second emulsifier includes no phospholipid component.

In some embodiments of the present application, the second emulsifier is one or more selected from egg yolk lecithin E80, glyceryl monooleate, polysorbate 80, sorbitan oleate 80, and sodium oleate. Further, the second emulsifier can be one or more selected from egg yolk lecithin E80, polysorbate 80, and sorbitan oleate 80.

The type and amount features of the second emulsifier can be combined in any suitable manner, including, but not limited to, the combinations specifically enumerated herein, which includes, but is not limited to, the combinations set forth in the embodiments given below.

### Stabilizer

In the present application, the EPA-EE nano-lipid composition optionally includes a stabilizer which can stabilize EPA and maintain the EPA concentration in blood, and therefore, can also be referred to as an EPA stabilizer. In the case of an oral formulation, after the oral absorption, the stabilizer becomes a constituent of chyle together with EPA. The stabilizer used in the present application can form a hydrated film on the surface of the chyle to mask the hydrophobic binding sites that interact with opsonins. The long-chain moiety (the polymer chain) in the stabilizer can form steric hindrance on the surface of the chyle, effectively avoiding the recognition and phagocytosis of the chyle by the reticuloendothelial system. Therefore, the prepared nano-lipid formulation can prolong the circulation time of EPA in blood, maintaining the effective drug concentration in blood, allowing EPA to exert the blood lipid-lowering effect for a long period, thereby achieving a significant treatment effect on atherosclerotic plaques. In contrast, a conventional EPA formulation has an inferior treatment effect on atherosclerotic plaques.

In some embodiments of the present application, the EPA-EE nano-lipid composition does not include the stabilizer.

In some embodiments of the present application, the EPA-EE nano-lipid composition includes the stabilizer, and the stabilizer is a non-ionic polymer, and further is an amphiphilic non-ionic polymer.

In some embodiments of the present application, the stabilizer is one or more selected from of a vitamin lipid polymer derivative, a phospholipid polymer derivative, a fatty acid ester polymer derivative, a polyoxyethylene-polyoxypropylene ether block copolymer, etc., all of which can achieve the aforementioned stabilization effect on EPA.

When a terminal group of the stabilizer is provided by a PEG unit, the terminal group provided by the PEG unit can be OH or methoxy. When the terminal group is methoxy, it may be referred to as mPEG.

In some embodiments of the present application, the non-ionic polymer is a polyethylene glycol derivative, and is further an amphiphilic polyethylene glycol derivative, wherein the molecular weight of the PEG unit is mainly dependent on various factors such as the stabilizing effect, the particle size of the formulation, and the drug release. In some embodiments of the present application, the non-ionic polymer is one or more selected from of a vitamin lipid polymer derivative, a phospholipid polymer derivative, a fatty acid ester polymer derivative, etc., and further, the molecular weight of the PEG unit is 200 Da to 6000 Da, and further can be 400 Da to 6000 Da. As non-limiting examples, the average molecular weight of the PEG unit is about 200 Da, 300 Da, 400 Da, 500 Da, 600 Da, 700 Da, 800 Da, 1000 Da, 1200 Da, 1300 Da, 1400 Da, 1500 Da, 1600 Da, 1800 Da, 2000 Da, 2200 Da, 2400 Da, 2500 Da, 2600 Da, 2800 Da, 3000 Da, 3200 Da, 3300 Da, 3400 Da, 3500 Da, 4000 Da, 4200 Da, 4400 Da, 4500 Da, 5000 Da, 5500 Da, 6000 Da, etc., and “about” indicates that there may be a variation within a certain range, such as +10%. For example, “about 1000” means 1000±10% (numerically equivalent to 9000 to 1100). The numerical values such as 2000, 200, 400, 600, 4000, 6000, etc. are the molecular weights of the PEG block, which can be number-average molecular weights or weight-average molecular weights.

In some embodiments of the present application, the vitamin unit in the vitamin lipid polymer derivative can independently and optionally be vitamin E. In some embodiments of the present application, the vitamin lipid polymer derivative is a vitamin lipid polyethylene glycol derivative. In some embodiments of the present application, the vitamin lipid polymer derivative is a vitamin E polyethylene glycol succinate (also known as a vitamin E succinic acid polyethylene glycol ester). In some embodiments of the present application, the molecular weight of the PEG unit in the vitamin lipid polymer derivative is 200 Da to 4000 Da. In some embodiments, non-limiting examples of the vitamin lipid polymer derivative are d-α-tocopheryl polyethylene glycol 200 succinate, d-α-tocopheryl polyethylene glycol 400 succinate, d-α-tocopheryl polyethylene glycol 1000 succinate, d-α-tocopheryl polyethylene glycol 1500 succinate, d-α-tocopheryl polyethylene glycol 2000 succinate, d-α-tocopheryl polyethylene glycol 4000 succinate, etc.

In the present application, “vitamin E polyethylene glycol succinate” and “vitamin E succinic acid polyethylene glycol ester” have the same meaning and can be used interchangeably.

In some embodiments of the present application, the phospholipid polymer derivative is a synthetic phospholipid modified with polyethylene glycol. Further, the molecular weight of the PEG unit in the phospholipid polymer derivative can be 400 Da to 6000 Da, such as 400 Da, 500 Da, 600 Da, 700 Da, 800 Da, 900 Da, 1000 Da, 1500 Da, 2000 Da, 2500 Da, 3000 Da, 3500 Da, 4000 Da, 5000 Da, 6000 Da, etc.

In some embodiments of the present application, the phospholipid unit in the phospholipid polymer derivative can independently and optionally includes a phosphatidylethanolamine unit.

In some embodiments of the present application, the phospholipid polymer derivative is selected from phosphatidylethanolamine-polyethylene glycol (PE-PEG), which can optionally contain a C₁₂ to C₂₀ fatty acyl group (e.g., a stearoyl group), and further can be a C₁₂ to C₂₀ fatty acyl phosphatidylethanolamine-polyethylene glycol. In some embodiments of the present application, the phospholipid polymer derivative is one or more selected from distearoyl phosphatidylethanolamine-polyethylene glycol (DSPE-PEG), dipalmitoyl phosphatidylethanolamine-methoxypolyethylene glycol (DPPE-mPEG), soybean phosphatidylethanolamine-polyethylene glycol monomethyl ether, 1,2-di-myristoyl-rac-glycero-3-methoxypolyethylene glycol, dilauroylphosphatidylethanolamine-polyethylene glycol, and dioleoyl phosphatidylethanolamine-polyethylene glycol. In some embodiments of the present application, the phospholipid polymer derivative is one or more selected from distearoyl phosphatidylethanolamine-polyethylene glycol 2000, distearoyl phosphatidylethanolamine-polyethylene glycol 5000, dipalmitoyl phosphatidylethanolamine-methoxypolyethylene glycol 2000, dipalmitoyl phosphatidylethanolamine-methoxypolyethylene glycol 5000, soybean phosphatidylethanolamine-polyethylene glycol monomethyl ether 2000, 1,2-dimyristoyl-rac-glycero-3-methoxypolyethylene glycol 2000, dilauroylphosphatidylethanolamine-polyethylene glycol 2000, and dioleoyl phosphatidylethanolamine-polyethylene glycol.

In some embodiments, the terminal group of the phospholipid polymer derivative is provided by the PEG unit, and the terminal group is OH or methoxy. In some embodiments, the fatty acid ester unit in the fatty acid ester polymer derivative can independently and optionally be a C₁₂ to C₂₀ fatty acid ester unit (having 12 to 20 carbon atoms, e.g., 12, 14, 16, 18, 20). A single molecule of any fatty acid ester unit can, independently, includes one, two, or more fatty acid chains, depending on factors such as the type of the ester. In some embodiments, the terminal group of the fatty acid ester polymer derivative is provided by the PEG unit, and the terminal group is OH or methoxy.

In some embodiments of the present application, the fatty acid ester polymer derivative is a fatty acid ester modified with polyethylene glycol. Further, the molecular weight of the PEG unit in the fatty acid ester polymer derivative can be 200 Da to 4000 Da, such as 200 Da, 300 Da, 400 Da, 500 Da, 600 Da, 700 Da, 800 Da, 900 Da, 1000 Da, 1500 Da, 2000 Da, 2500 Da, 3000 Da, 3500 Da, 4000 Da, etc.

In some embodiments of the present application, the fatty acid ester polymer derivative is one or more selected from polyethylene glycol-C₁₂ to C₂₀ fatty acid ester, polyethylene glycol-di-C₁₂ to C₂₀ fatty acid ester, etc. In some embodiments of the present application, the fatty acid ester polymer derivative is one or more selected from polyethylene glycol 400 oleate, polyethylene glycol 600 oleate, polyethylene glycol 4000 oleate, polyethylene glycol 6000 oleate, polyethylene glycol 400 dioleate, polyethylene glycol 600 dioleate, polyethylene glycol 200 laurate, polyethylene glycol 200 dilaurate, polyethylene glycol 400 laurate, polyethylene glycol 400 dilaureate, polyethylene glycol 400 stearate, polyethylene glycol 400 distearate, etc.

In some embodiments of the present application, the polyoxyethylene-polyoxypropylene ether block copolymer is a two-block copolymer. In some embodiments of the present application, the average molecular weight of the polyoxyethylene-polyoxypropylene ether block copolymer is 3000 Da to 10000 Da, such as 3500 Da, 8350 Da, etc. In some embodiments of the present application, the mass content of polyoxyethylene blocks is 50% to 80%. In some embodiments of the present application, the polyoxyethylene-polyoxypropylene ether block copolymer is poloxamer, which is commercially available, and further, the poloxamer can be Pluronic L65 (the content of polyoxyethylene is 50%, the average molecular weight is 3500 Da), Pluronic F68 (the content of polyoxyethylene is 80%, the average molecular weight is 8350 Da), etc. In some embodiments of the present application, the stabilizer includes the polyoxyethylene-polyoxypropylene ether block copolymer. In some embodiments of the present application, the stabilizer is the polyoxyethylene-polyoxypropylene ether block copolymer.

In some embodiments, the EPA-EE nano-lipid composition is a PEG-modified lipid composition, and further, the weight percentage of the PEG-modified raw material in the EPA-EE nano-lipid composition is 0.01% to 10%, such as 0.05%, 0.1%, 0.15%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, etc.

In some embodiments of the present application, the weight percentage of the stabilizer in the EPA-EE nano-lipid composition is 0 to 5% (w/w), which can further be 0% to 5% (w/w), which can further be 0.1% to 3% (w/w), and for example, can be any one of the following percentages or in a percentage range formed between any two of the following percentages: 0%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, 3.5%, 3.5% 4%, 4.2%, 4.5%, 4.6%, 4.8%, 5%, etc., such as 0% to 5% (w/w).

In some embodiments of the present application, the stabilizer is one or more of TPGS, DSPE-PEG, or S40. In some embodiments, the stabilizer is a combination of TPGS and S40, and further can be a mixture of TPGS and S40 in equal masses.

The type and amount features of the stabilizer can be combined in suitable manner, including, but not limited to, the combinations specifically enumerated herein, which includes, but are not limited to, the combinations set forth in the embodiments given below.

### First Auxiliary Material

In the present application, the EPA-EE nano-lipid composition optionally includes a lipoprotein binding promoter, also referred to as a first auxiliary material. The first auxiliary material can promote EPA to bind with a lipoprotein, and is a competitive lipoprotein binding auxiliary material. The first auxiliary material can interact with positively charged residues at the polar-nonpolar interface of the amphiphilic helix of the lipoprotein to promote EPA therein to bind with the low-density lipoprotein. In the present application, increasing the content of the EPA in the lipoprotein and decreasing the content of the saturated fatty acid in the lipoprotein are both conducive to reducing the formation of oxidized lipoproteins, reducing the accumulation of saturated fatty acids in the inner wall of the blood vessel, reducing the damage of endothelial cells, and enhancing the treatment effect on atherosclerosis.

In some embodiments of the present application, the EPA-EE nano-lipid composition does not include the first auxiliary material.

In some embodiments of the present application, the first auxiliary material is one or more selected from an amino acid with a side chain having a negatively charged group, an amino acid derivative with a negatively charged group, a small peptide with a side chain having a negatively charged group, etc. In some embodiments, the amino acid with the side chain having the negatively charged group is one or more selected from aspartic acid, glutamic acid, taurine, etc. In some embodiments, the amino acid derivative with the side chain having the negatively charged group is one or more selected from phosphatidylserine, dihexadecyl-glutamate-glutamine, dihexadecyl-glutamate-glutamic acid, dihexadecyl-glutamate-asparagine, etc. In some embodiments, the small peptide with the side chain having the negatively charged group is selected from glutathione.

In some non-limiting embodiments of the present application, the first auxiliary material is one or more selected from aspartic acid, glutamic acid, taurine, phosphatidylserine, dihexadecyl-glutamate-glutamine, dihexadecyl-glutamate-glutamic acid, dihexadecyl-glutamate-asparagine, glutathione, etc.

In some embodiments of the present application, the weight percentage of the first auxiliary material in the EPA-EE nano-lipid composition is 0 to 5% (w/w), further can be 0.1% to 5% (w/w), and yet further can be 0.1% to 3% (w/w), and for example, can be any one of the following percentages or in a percentage range formed between any two of the following percentages: 0%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, 3.5%, 4%, 4.2%, 4.5%, 4.6%, 4.8%, 5%, etc., such as 0.1% to 5% (w/w).

In some embodiments of the present application, the first auxiliary material is one or more selected from phosphatidylserine, sodium glutamate, and taurine.

In some embodiments of the present application, the first auxiliary material is a combination of taurine and sodium glutamate, and further can be a mixture of taurine and sodium glutamate in equal masses.

In some embodiments of the present application, the EPA-EE nano-lipid composition includes both the aforementioned stabilizer and the aforementioned first auxiliary material. In this case, through the synergy between the raw material containing the high content of EPA-EE, the stabilizer, and the first auxiliary material, the formulation of the composition can maintain the EPA concentration in plasma, increase the exposure level of EPA, and promote the binding of EPA with the low density lipoprotein, which can improve the blood lipid-lowering and atherosclerosis treatment effects of EPA and achieve significant treatment effects which the existing reported formulations cannot reach.

In some embodiments of the present application, the content of the stabilizer is 0.1% to 5% (w/w) based on the total weight of the EPA-EE nano-lipid composition. Exemplary contents of the stabilizer can refer to the above description.

In some embodiments of the present application, the content of the first auxiliary material is 0.1% to 5% (w/w) based on the total weight of the EPA-EE nano-lipid composition. Exemplary contents of the first auxiliary material can refer to the above description.

In some embodiments of the present application, the content of the stabilizer is 0.1% to 5% (w/w) and the content of the first auxiliary material is 0.1% to 5% (w/w) based on the total weight of the EPA-EE nano-lipid composition. Exemplary contents of the stabilizer and the first auxiliary material can refer to the above description.

The type and amount features of the first auxiliary material can be combined in a suitable manner, including but not limited to the combinations specifically enumerated herein, which includes, but is not limited to, the combinations set forth in the embodiments given below.

### Second Auxiliary Material

In the present application, the EPA-EE nano-lipid composition optionally further includes an additional auxiliary material (referred to as the second auxiliary material) other than the first auxiliary material.

The second auxiliary material in the present application is different from any one of the first emulsifier, the second emulsifier, the stabilizer, and the first auxiliary material.

In some embodiments of the present application, the second auxiliary material includes one or more selected from an antioxidant, a base oil, a flavoring agent, an interfacial membrane stabilizer, a pH adjuster, etc.

In some embodiments of the present application, the second auxiliary material includes one or more selected from a base oil (mainly referring to an oil other than EPA and its derivatives), an antioxidant, a co-emulsifier, a pH adjuster, a thickener, a flavoring agent, etc.

In some embodiments of the present application, the weight percentage of the second auxiliary material in the EPA-EE nano-lipid composition is 0 to 15% (w/w), and further can be 0.01% to 10% (w/w), and for example, can be any one of the following percentages or in a percentage range formed between any two of the following percentages: 0.01%, 0.02%, 0.03%, 0.04%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.2%, 2.4%, 2.5%, 2.6%, 2.8%, 3%, 3.5%, 4%, 4.2%, 4.5%, 4.6%, 4.8%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, etc., such as 0.01% to 15% (w/w).

In some embodiments of the present application, the EPA-EE nano-lipid composition optionally includes an antioxidant. In some embodiments of the present application, the antioxidant is one or more selected from vitamin E, α-tocopherol, β-tocopherol, γ-tocopherol, mixed tocopherols, α-tocopheryl acetate, β-tocopheryl acetate, γ-tocopheryl acetate, mixed tocopheryl acetates, ascorbic acid (vitamin C), ascorbyl palmitate, ascorbyl stearate, ascorbyl myristate, sodium ascorbate, butylated hydroxyanisole (BHA), dibutylated hydroxytoluene (BHT), propylgallate (PG), tert-butylhydroquinone (TBHQ), etc. In some embodiments of the present application, the antioxidant is one or more selected from vitamin E, α-tocopherol, γ-tocopherol, mixed tocopherols, α-tocopheryl acetate, γ-tocopheryl acetate, mixed tocopheryl acetates, ascorbic acid (Vitamin C), ascorbyl palmitate, ascorbyl stearate, ascorbyl myristate, sodium ascorbate, butylated hydroxyanisole (BHA), dibutylated hydroxytoluene (BHT), propylgallate (PG), tert-butylhydroquinone (TBHQ), etc. In some embodiments of the present application, the mass content of the antioxidant in the EPA-EE nano-lipid composition is 0 to 1%, such as 0%, 0.1%, 0.2%, 0.3%, 0.4%, 0.5%, 0.6%, 0.7%, 0.8%, 0.9%, 1%, etc. In some embodiments, the antioxidant is α-tocopherol.

In some embodiments of the present application, the EPA-EE nano-lipid composition optionally includes a base oil. In some embodiments of the present application, the base oil refers primarily to an oil other than EPA and its derivatives. In some embodiments of the present application, the base oil is one or more selected from soybean oil, olive oil, jojoba oil, sweet almond oil, grapeseed oil, corn oil, walnut oil, sea buckthorn oil, olive oil, coix seed oil, grapeseed oil, ginger oil, coconut oil, camellia oil, rose oil, peppermint oil, lemon oil, medium-chain triglycerides (e.g., glycerides of C₈₋₁₀ fatty acids), etc. In some embodiments of the present application, the mass content of the base oil in the EPA-EE nano-lipid composition is 0% to 1%, such as 0%, 0.1%, 0.2%, 0.3%, 0.4%, 0.5%, 0.6%, 0.7%, 0.8%, 0.9%, 1%, etc.

In some embodiments, the base oil is one or more selected from corn oil, olive oil, and medium-chain triglycerides. In some embodiments, the base oil is corn oil, olive oil, or a combination thereof. In some embodiments, the base oil is a combination of corn oil and olive oil, and further, can be a mixture of corn oil and olive oil in equal masses. In some embodiments, the base oil is olive oil or a medium-chain triglyceride. In some embodiments, the base oil is a medium-chain triglyceride. Reference can be made to the individual formulations in Tables 2-4.

In some embodiments of the present application, the second auxiliary material includes the antioxidant and the base oil, and further, can be a combination of the antioxidant and the base oil. The types and the amounts of the antioxidant and the base oil can be respectively as defined in any suitable embodiments.

In some embodiments, the second auxiliary material is a combination of an antioxidant and a base oil. Further, the antioxidant is α-tocopherol, and the base oil is one or more selected from corn oil, olive oil, and a medium-chain triglyceride. In some of these embodiments, the base oil is corn oil, olive oil, or a combination thereof, is further a combination of corn oil and olive oil, and is yet further a mixture of corn oil and olive oil in equal masses. In some other of these embodiments, the base oil is olive oil or a medium-chain triglyceride. In some other of these embodiments, the second auxiliary material is a combination of α-tocopherol and a medium-chain triglyceride.

In some embodiments of the present application, the EPA-EE nano-lipid composition optionally includes a co-emulsifier. In some embodiments of the present application, the co-emulsifier is one or more selected from casein, sodium caseinate, sodium polyacrylate, etc.

In some embodiments of the present application, the EPA-EE nano-lipid composition optionally includes a pH adjuster. The pH adjuster is primarily configured to adjust the pH condition of the aqueous phase in preparation of the EPA-EE nano-lipid composition. In some embodiments of the present application, the pH adjuster is one or more selected from citric acid, sodium citrate, potassium citrate, acetic acid, sodium acetate, phosphoric acid, phosphate, hydrochloric acid, citric acid, sodium citrate, lactic acid, tartaric acid, malic acid, DL-malic acid, fumaric acid, metatartaric acid, L(+)-tartaric acid, glacial acetic acid, acetic acid, adipic acid, monosodium fumarate, calcium lactate, sodium acetate, calcium hydroxide, potassium hydroxide, sodium hydroxide, etc.

In some embodiments of the present application, the EPA-EE nano-lipid composition optionally includes an interfacial membrane stabilizer. In some embodiments of the present application, the interfacial membrane stabilizer is one or more selected from glycerol, propylene glycol, mannitol, oleic acid, sodium oleate, cholesterol, etc.

In some embodiments of the present application, the EPA-EE nano-lipid composition optionally includes a thickener. In some embodiments of the present application, the thickener is one or more selected from carrageenan, xanthan gum, carbomer, etc.

In some embodiments of the present application, the EPA-EE nano-lipid composition optionally includes a flavoring agent. In some embodiments of the present application, the flavoring agent is one or more selected from sucrose, fructose, sucralose, neotame, erythritol, mogrosides, natural flavors, natural perfumes, menthol, etc.

In some embodiments of the present application, an EPA-EE nano-lipid composition includes EPA-EE (in high concentration, provided by a high-purity EPA-EE raw material), a stabilizer having the effect of maintaining the EPA concentration in blood, a first auxiliary material having the effect of facilitating the binding of the EPA with the lipoprotein, an emulsifier, the antioxidant, and other auxiliary materials for regulating the mouthfeel and taste of the nano-lipid formulation.

In some embodiments of the present application, an EPA-EE nano-lipid composition includes the following components, based on the total weight of the EPA-EE nano-lipid composition: 4% to 20% (w/w) of EPA-EE, 0.1% to 10% (w/w) of a first emulsifier, 0.01% to 10% (w/w) of a second emulsifier, and water as balance to 100% (w/w). Further, the following components can be included: 0% to 5% (w/w) of a stabilizer (having the effect of maintaining the EPA concentration in blood), 0% to 5% (w/w) of a first auxiliary material (having the effect of facilitating the binding of EPA with the lipoprotein), and 0% to 15% (w/w) of a second auxiliary material. The second auxiliary material can be one or more of additional food-acceptable or pharmaceutically acceptable raw or auxiliary materials, and optionally is, a pharmaceutically acceptable auxiliary material. As a non-limiting example, the EPA-EE nano-lipid composition includes 0% to 5% (w/w) of an antioxidant.

The type and amount features of the second auxiliary material can be combined in suitable manners, including, but not limited to, the combinations specifically enumerated herein, which includes, but is not limited to, the combinations set forth in the embodiments below.

### Water

In the present application, the EPA-EE nano-lipid composition necessarily includes water as a solvent, so that the EPA-EE nano-lipid composition can be prepared into a water-based formulation that can be easily administered to a patient.

In the present application, the water in the EPA-EE nano-lipid composition can be deionized water, distilled water, sterile water, etc., as long as it is suitable for the preparation of the pharmaceutical formulation. The water is present in the EPA-EE nano-lipid composition in an appropriate amount. The amount of water is at least 65% (w/w). The appropriate amount of water ensures a suitable oil-to water phase ratio in the EPA-EE nano-lipid composition such that an oil-in-water structure can be formed. In some embodiments of the present application, the weight percentage of water in the EPA-EE nano-lipid composition can be, for example, any one of the following percentages or in a percentage range formed between any two of the following percentages: 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 89.1%, 89.2%, 89.3%, 89.4%, 89.5%, 89.6%, 89.7%, 89.8%, etc.

In the present application, the features such as the type and amount of the EPA-EE raw material, the type and amount of the first emulsifier, the type and amount of the second emulsifier, the type and amount of the stabilizer, the type and amount of the first auxiliary material, the type and amount of the second auxiliary material, and the type and amount of the water can be combined in any suitable manners, including, but not limited to, the combinations specifically enumerated herein, which include, but are not limited to, the combinations set forth in the embodiments below.

### Some Embodiments

In some embodiments of the present application, based on the total weight of the EPA-EE nano-lipid composition, the EPA-EE nano-lipid composition includes the following components in weight percentages:
the EPA-EE raw material 1% to 30% (w/w);
the first emulsifier 0.1% to 10% (w/w);
the second emulsifier 0.01% to 10% (w/w);
the stabilizer 0% to 5% (w/w);
the first auxiliary material 0% to 5% (w/w);
the second auxiliary material 0% to 15% (w/w); and
water (an appropriate amount of water, which for example, includes, but is not limited to, those described above).

In some embodiments of the present application, based on the total weight of the EPA-EE nano-lipid composition, the EPA-EE nano-lipid composition includes the following components in weight percentages:
the EPA-EE raw material 4% to 20% (w/w);
the first emulsifier 0.5% to 5% (w/w);
the second emulsifier 0% to 10% (w/w);
the stabilizer 0.1% to 3% (w/w);
the first auxiliary material 0.1% to 3% (w/w);
the second auxiliary material 0.01% to 10% (w/w); and
water (an appropriate amount of water, which, for example, includes, but is not limited to, those described above).

Furthermore, the EPA-EE nano-lipid composition can achieve a maximum blood drug concentration greater than 700 μg/mL within 2 hours in a rat upon oral administration at a dose of 400 mg/kg.

In some embodiments of the present application, the EPA-EE nano-lipid composition satisfies at least one feature from groups (i) and (ii), at least one feature from group (i), or at least one feature from group (ii):
(i) based on the total weight of the EPA-EE nano-lipid composition, the content of the stabilizer is 0.01% to 5%, optionally 0.05% to 5% (w/w), further optionally 0.05% to 2% (w/w), yet further optionally 0.05% to 1% (w/w), yet further optionally 0.1% to 0.2% (w/w); alternatively, the content of the stabilizer is optionally 0.1% to 5% (w/w), further optionally 0.1% to 2% (w/w), yet further optionally 0.1% to 1% (w/w);
(ii) based on the total weight of the EPA-EE nano-lipid composition, the content of the first auxiliary material is 0.01% to 5% (w/w), optionally, 0.05% to 5% (w/w), further optionally 0.05% to 2% (w/w), yet further optionally 0.05% to 1% (w/w), yet further optionally 0.1% to 0.2% (w/w); alternatively, the content of the first auxiliary material is optionally 0.1% to 5% (w/w), further optionally 0.1% to 2% (w/w), yet further optionally 0.1% to 1% (w/w).

In some embodiments of the present application, the EPA-EE nano-lipid composition satisfies any one or any combination of the following features:
based on the total weight of the EPA-EE nano-lipid composition, the weight proportion of the second emulsifier is 0.1% to 10%, optionally 0.2% to 8%, further optionally 0.4% to 8%, yet further optionally 0.4% to 6%, yet further optionally 0.5% to 5%, yet further optionally 1% to 5%, yet further optionally 1% to 2%; or the weight proportion of the second emulsifier is 0.2% to 10%, further optionally 0.2% to 6%, yet further optionally 0.2% to 5%, yet further optionally 0.2% to 2%; or the weight proportion of the second emulsifier is 0.5% to 10%, further optionally 0.5% to 8%, yet further optionally 0.5% to 6%, yet further optionally 0.5% to 2%, yet further optionally 0.5% to 1.5%; or the weight proportion of the second emulsifier is 1% to 10%, further optionally 1% to 8%, yet further optionally 1% to 6%, yet further optionally 1% to 5%;
based on the total weight of the EPA-EE nano-lipid composition, the weight proportion of the stabilizer is 0.1% to 5%, optionally 0.1% to 4%, further optionally 0.1% to 3%, yet further optionally 0.1% to 2%, yet further optionally 0.2% to 2%, yet further optionally 0.4% to 2%, yet further optionally 0.5% to 2%, yet further optionally 1% to 2%; or the weight proportion of the stabilizer is 0.2% to 4%, optionally 0.2% to 3%; or the weight proportion of the stabilizer is 0.4% to 4%, optionally 0.4% to 3%; or the weight proportion of the stabilizer is 0.5% to 4%, optionally 0.5% to 3%; or the weight proportion of the stabilizer is 1% to 4%, optionally 1% to 3%;
based on the total weight of the EPA-EE nano-lipid composition, the weight proportion of the first auxiliary material is 0.1% to 5%, optionally 0.1% to 4%, further optionally 0.1% to 3%, yet further optionally 0.1% to 2%, yet further optionally 0.2% to 2%, yet further optionally 0.4% to 2%, yet further optionally 0.5% to 2%, yet further optionally 1% to 2%; or the weight proportion of the stabilizer is 0.2% to 4%, optionally 0.2% to 3%; or the weight proportion of the stabilizer is 0.4% to 4%, optionally 0.4% to 3%; or the weight proportion of the stabilizer is 0.5% to 4%, optionally 0.5% to 3%; or the weight proportion of the stabilizer is 1% to 4%, optionally 1% to 3%;
based on the total weight of the EPA-EE nano-lipid composition, the weight proportion of the second auxiliary material is 0.01% to 15%, optionally 0.01% to 10%, further optionally 0.01% to 8%, yet further optionally 0.01% to 6%, yet further optionally 0.01% to 5.5%, yet further optionally 0.1% to 5.5%, yet further optionally 0.5% to 5.5%, yet further optionally 1% to 5.5%, yet further optionally 2% to 5.5%, yet further optionally 3% to 5.5%; or the weight proportion of the second auxiliary material is 0.1% to 15%, yet further optionally 0.1% to 10%, yet further optionally 0.1% to 8%, yet further optionally 0.1% to 6%, yet further optionally 0.1% to 5%, yet further optionally 0.1% to 4%; or the weight proportion of the second auxiliary material is 0.5% to 15%, yet further optionally 0.5% to 10%, yet further optionally 0.5% to 8%, yet further optionally 0.5% to 6%, yet further optionally 0.5% to 5%, yet further optionally 0.5% to 4%; or the weight proportion of the second auxiliary material is 1% to 15%, yet further optionally 1% to 10%, yet further optionally 1% to 8%, yet further optionally 1% to 6%, yet further optionally 1% to 5%, yet further optionally 1% to 4%; or the weight proportion of the second auxiliary material is 2% to 15%, yet further optionally 2% to 10%, yet further optionally 2% to 8%, yet further optionally 2% to 6%, yet further optionally 2% to 5%, yet further optionally 2% to 4%; or the weight proportion of the second auxiliary material is 3% to 15%, yet further optionally 3% to 10%, yet further optionally 3% to 8%, yet further optionally 3% to 6%, yet further optionally 3% to 5%, yet further optionally 3% to 4%.

In some embodiments of the present application, the EPA-EE nano-lipid composition satisfies any one or any combination of the following features:
the EPA-EE raw material is an ethyl esterification product of one or more oils selected from a deep-sea fish oil, a seaweed oil, a krill oil, etc.;
the mass content of EPA-EE in the EPA-EE raw material is greater than or equal to 70%, optionally greater than or equal to 80%; or the mass content of EPA-EE in the EPA-EE raw material is 60% to 97%, optionally 70% to 97%, further optionally 80% to 97%;
the iodine value of the highly unsaturated phospholipid is greater than or equal to 80, optionally greater than or equal to 85, further optionally greater than or equal to 95; or the iodine value of the highly unsaturated phospholipid is 80 to 113, optionally 85 to 113, further optionally 90 to 113, yet further optionally 95 to 113; or the iodine value of the highly unsaturated phospholipid is 70 to 113, optionally 70 to 110, further optionally 70 to 105, yet further optionally 70 to 102, yet further optionally 70 to 100, yet further optionally 70 to 95, yet further optionally 70 to 90; or the iodine value of the highly unsaturated phospholipid is 80 to 113, optionally 80 to 110, further optionally 80 to 105, yet further optionally 80 to 102, yet further optionally 80 to 100, yet further optionally 80 to 97, yet further optionally 80 to 97, yet further optionally 80 to 95; or the iodine value of the highly unsaturated phospholipid is 85 to 113, optionally 85 to 110, further optionally 85 to 105, yet further optionally 85 to 102, yet further optionally 85 to 100, yet further optionally 85 to 97; or the iodine value of the highly unsaturated phospholipid is 90 to 113, optionally 90 to 110, further optionally 90 to 105, yet further optionally 90 to 102, yet further optionally 90 to 97, yet further optionally 94 to 97;
the mass proportion of phosphatidylcholine in the highly unsaturated phospholipid is greater than or equal to 60%, optionally greater than or equal to 70%; or the mass proportion of phosphatidylcholine in the highly unsaturated phospholipid is 50% to 98%, optionally 60% to 98%, further optionally 70% to 98%; or the mass proportion of phosphatidylcholine in the highly unsaturated phospholipid is 50% to 94%, optionally 60% to 94%, further optionally 70% to 94%;
the first emulsifier is one or more selected from soybean phospholipid, sunflower seed phospholipid, and polyene phosphatidylcholine;
the stabilizer is an amphiphilic non-ionic polymer, and the stabilizer is one or more selected from a vitamin lipid polymer derivative, a phospholipid polymer derivative, a fatty acid ester polymer derivative, and a polyoxyethylene-polyoxypropylene ether block copolymer;
optionally, the vitamin lipid polymer derivative is a vitamin E polyethylene glycol succinate (i.e., a vitamin E succinic acid polyethylene glycol ester);
optionally, the phospholipid polymer derivative is a polyethylene glycol-modified synthetic phospholipid;
optionally, the fatty acid ester polymer derivative is a polyethylene glycol-modified fatty acid ester;
the molecular weight of the polyethylene glycol (PEG) unit in the phospholipid polymer derivative is 400 Da to 6000 Da, optionally 1000 Da to 6000 Da, further optionally 1000 Da to 5000 Da, yet further optionally 1000 Da to 4000 Da, yet further optionally 1000 Da to 3500 Da, yet further optionally 1000 Da to 3000 Da, yet further optionally 1500 Da to 2500 Da, yet further optionally 1600 Da to 2400 Da, yet further optionally 1800 Da to 2200 Da; or the molecular weight of the PEG unit in the phospholipid polymer derivative is 400 Da to 5000 Da, optionally 4000 Da to 4000 Da, further optionally 400 Da to 3000 Da, yet further optionally 400 Da to 2000 Da; the molecular weight refers to the number-average molecular weight or the weight-average molecular weight, optionally the weight-average molecular weight;
the molecular weight of the PEG unit in the fatty acid ester polymer derivative is 200 Da to 4000 Da, optionally 1000 Da to 6000 Da, further optionally 1000 Da to 5000 Da, yet further optionally 1000 Da to 4000 Da, yet further optionally 1000 Da to 3500 Da, yet further optionally 1000 Da to 3000 Da, yet further optionally 1500 Da to 2500 Da, yet further optionally 1600 Da to 2400 Da, yet further optionally 1800 Da to 2200 Da; or the molecular weight of the PEG unit in the fatty acid ester polymer derivative is 400 Da to 5000 Da, optionally 400 Da to 4000 Da, further optionally 400 Da to 3000 Da, yet further optionally 400 Da to 2000 Da; the molecular weight refers to the number-average molecular weight or the weight-average molecular weight, optionally the weight-average molecular weight;
the first auxiliary material is one or more selected from an amino acid with a side chain having a negatively charged group, an amino acid derivative with a negatively charged group, and a small peptide with a side chain having a negatively charged group;
the second auxiliary material is one or more selected from an antioxidant, a base oil, a co-emulsifier, a flavoring agent, an interfacial membrane stabilizer, a thickener, and a pH adjuster;
the EPA-EE nano-lipid composition is in form of submicron emulsion containing droplets with an average particle size less than or equal to 500 nm, optionally 10 nm to 500 nm, further optionally 100 nm to 500 nm, yet further optionally 100 nm to 300 nm, yet further optionally 150 nm to 250 nm, or the average particle size of the droplets is less than or equal to 300 nm, optionally less than or equal to 250 nm.

In some embodiments of the present application, the EPA-EE nano-lipid composition satisfies any one or any combination of the following features:
the iodine value of the first emulsifier is greater than 90, and the first emulsifier is one or more selected from soybean phospholipid, sunflower seed phospholipid, and polyene phosphatidylcholine;
the second emulsifier is one or more selected from a phospholipid different from the first emulsifier, sucrose ester, citric and fatty acid esters of glycerol, fatty acid glyceride, glyceryl monolinoleate, glycerol monostearate, polysorbate, sorbitan fatty acid ester, polyoxyethylene fatty acid ester, Span, alginate, sodium oleate, and caseinate; optionally, the second emulsifier is one or more selected from a phospholipid different from the first emulsifier, sucrose ester, citric acid and acid esters of glycerol, fatty acid glyceride, polysorbate, fatty acid sorbitan, polyoxyethylene fatty acid ester, Span, alginate, and caseinate;
the stabilizer includes a terminal group provided by a PEG unit, and the terminal group is OH or methoxy;
the vitamin lipid polymer derivative is one or more selected from d-α-tocopheryl polyethylene glycol 200 succinate, d-α-tocopheryl polyethylene glycol 400 succinate, d-α-tocopheryl polyethylene glycol 1000 succinate, d-α-tocopheryl polyethylene glycol 1500 succinate, d-α-tocopheryl polyethylene glycol 2000 succinate, and d-α-tocopheryl polyethylene glycol 4000 succinate;
the phospholipid polymer derivative is selected from distearoylphosphatidylethanolamine-polyethylene glycol 2000, distearoylphosphatidylethanolamine-polyethylene glycol 5000, dipalmitoylphosphatidylethanolamine-methoxypolyethylene glycol 2000, dipalmitoylphosphatidylethanolamine-methoxypolyethylene glycol 5000, soybean phosphatidylethanolamine-polyethylene glycol monomethyl ether 2000, 1,2-di-myristoyl-rac-glycero-3-methoxypolyethylene glycol 2000, dilauroylphosphatidylethanolamine-polyethylene glycol 2000, and dioleoylphosphatidylethanolamine-polyethylene glycol;
the fatty acid ester polymer derivative is one or more selected from polyethylene glycol 400 oleate, polyethylene glycol 600 oleate, polyethylene glycol 4000 oleate, polyethylene glycol 6000 oleate, polyethylene glycol 400 dioleate, polyethylene glycol 600 dioleate, polyethylene glycol 200 laurate, polyethylene glycol 200 dilaurate, polyethylene glycol 400 laurate, polyethylene glycol 400 dilaurate, polyethylene glycol 400 stearate, and polyethylene glycol 400 distearate;
the polyoxyethylene-polyoxypropylene ether block copolymer is one or more selected from Pluronic L65 and Pluronic F68;
in the first auxiliary material, the amino acid with the side chain having the negatively charged group is one or more selected from aspartic acid, glutamic acid, and taurine;
in the first auxiliary material, the amino acid derivative with the side chain having the negatively charged group is one or more selected from phosphatidylserine, dihexadecyl-glutamate-glutamine, dihexadecyl-glutamate-glutamic acid, dihexadecyl-glutamate-asparagine;
in the first auxiliary material, the small peptide with the side chain having the negatively charged group is glutathione;
in the second auxiliary material, the antioxidant is one or more selected from vitamin E, α-tocopherol, β-tocopherol, γ-tocopherol, mixed tocopherols, α-tocopheryl acetate, β-tocopheryl acetate, γ-tocopheryl acetate, mixed tocopheryl acetates, ascorbic acid, ascorbyl palmitate, ascorbyl stearate, ascorbyl myristate, sodium ascorbate, butylated hydroxyanisole, dibutylated hydroxytoluene, propylgallate, and tert-butylhydroquinone;
in the second auxiliary material, the base oil is one or more selected from soybean oil, olive oil, jojoba oil, sweet almond oil, grapeseed oil, corn oil, walnut oil, sea buckthorn oil, olive oil, coix seed oil, grapeseed oil, ginger oil, coconut oil, camellia oil, rose oil, peppermint oil, lemon oil, medium-chain triglycerides, etc.

In some embodiments of the present application, the EPA-EE nano-lipid composition satisfies any one or any combination of the following features:
the EPA-EE raw material is an ethyl esterification product of one or more oils selected from a deep-sea fish oil, a seaweed oil, and a krill oil;
the mass content of EPA-EE in the EPA-EE raw material is greater than or equal to 70%, optionally greater than or equal to 80%; or the mass content of EPA-EE in the EPA-EE raw material is 60% to 97%, optionally 70% to 97%, further optionally 80% to 97%;
the iodine value of the highly unsaturated phospholipid is greater than or equal to 80, optionally greater than or equal to 85, further optionally greater than or equal to 90, further optionally greater than or equal to 95; or the iodine value of the highly unsaturated phospholipid is 80 to 113, optionally 85 to 113, further optionally 90 to 113, yet further optionally 95 to 113; or the iodine value of the highly unsaturated phospholipid is 70 to 113, optionally 70 to 110, further optionally 70 to 105, yet further optionally 70 to 102, yet further optionally 70 to 100, yet further optionally 70 to 95, yet further optionally 70 to 90; or the iodine value of the highly unsaturated phospholipid is 80 to 113, optionally 80 to 110, further optionally 80 to 105, yet further optionally 80 to 102, yet further optionally 80 to 100, yet further optionally 80 to 97, yet further optionally 80 to 97, yet further optionally 80 to 95; or the iodine value of the highly unsaturated phospholipid is 85 to 113, optionally 85 to 110, further optionally 85 to 105, further optionally 85 to 102, yet further optionally 85 to 100, yet further optionally 85 to 97; or the iodine value of the highly unsaturated phospholipid is 90 to 113, optionally 90 to 110, further optionally 90 to 105, further optionally 90 to 102, yet further optionally 90 to 97, yet further optionally 94 to 97;
the mass proportion of phosphatidylcholine in the highly unsaturated phospholipid is greater than or equal to 60%, optionally greater than or equal to 70%; or the mass proportion of phosphatidylcholine in the highly unsaturated phospholipid is 50% to 98%, optionally 60% to 98%, further optionally 70% to 98%; or the mass proportion of phosphatidylcholine in the highly unsaturated phospholipid is 50% to 94%, optionally 60% to 94%, further optionally 70% to 94%;
the first emulsifier is one or more selected from soybean phospholipid, sunflower seed phospholipid, and polyene phosphatidylcholine;
the stabilizer is an amphiphilic non-ionic polymer, and the stabilizer is one or more selected from a vitamin lipid polymer derivative, a phospholipid polymer derivative, a fatty acid ester polymer derivative, and a polyoxyethylene-polyoxypropylene ether block copolymer;
optionally, the vitamin lipid polymer derivative is a vitamin E polyethylene glycol succinate;
optionally, the phospholipid polymer derivative is a polyethylene glycol-modified synthetic phospholipid;
optionally, the fatty acid ester polymer derivative is a polyethylene glycol-modified fatty acid ester;
the molecular weight of the PEG unit in the phospholipid polymer derivative is 400 Da to 6000 Da, optionally 1000 Da to 6000 Da, further optionally 1000 Da to 5000 Da, yet further optionally 1000 Da to 4000 Da, yet further optionally 1000 Da to 3500 Da, yet further optionally 1000 Da to 3000 Da, yet further optionally 1500 Da to 2500 Da, yet further optionally 1600 Da to 2400 Da, yet further optionally 1800 Da to 2200 Da; or the molecular weight of the PEG unit in the phospholipid polymer derivative is 400 Da to 5000 Da, optionally 4000 Da to 4000 Da, further optionally 400 Da to 3000 Da, yet further optionally 400 Da to 2000 Da; the molecular weight refers to the number-average molecular weight or the weight-average molecular weight, optionally the weight-average molecular weight;
the molecular weight of the PEG unit in the fatty acid ester polymer derivative is 200 Da to 4000 Da, optionally 1000 Da to 6000 Da, further optionally 1000 Da to 5000 Da, yet further optionally 1000 Da to 4000 Da, yet further optionally 1000 Da to 3500 Da, yet further optionally 1000 Da to 3000 Da, yet further optionally 1500 Da to 2500 Da, yet further optionally 1600 Da to 2400 Da, yet further optionally 1800 Da to 2200 Da; or the molecular weight of the PEG unit in the fatty acid ester polymer derivative is 400 Da to 5000 Da, optionally 400 Da to 4000 Da, further optionally 400 Da to 3000 Da, yet further optionally 400 Da to 2000 Da; the molecular weight refers to the number-average molecular weight or the weight-average molecular weight, optionally the weight-average molecular weight;
the first auxiliary material is one or more selected from an amino acid with a side chain having a negatively charged group, an amino acid derivative with a negatively charged group, and a small peptide with a side chain having a negatively charged group;
the second auxiliary material is one or more selected from an antioxidant, a base oil, a co-emulsifier, a flavoring agent, an interfacial membrane stabilizer, a thickener, and a pH adjuster; and
the EPA-EE nano-lipid composition is in form of submicron emulsion containing droplets with an average particle size less than or equal to 500 nm, optionally 10 nm to 500 nm, further optionally 100 nm to 500 nm, yet further optionally 100 nm to 300 nm, yet further optionally 150 nm to 250 nm; or the average particle size of the droplets is less than or equal to 300 nm, optionally less than or equal to 250 nm.

In some embodiments, the EPA-EE nano-lipid composition satisfies any one or any combination of the following features:
the iodine value of the first emulsifier is greater than 90, and the first emulsifier is one or more selected from soybean phospholipid, sunflower seed phospholipid, and polyene phosphatidylcholine;
the second emulsifier is one or more selected from a phospholipid different from the first emulsifier, sucrose ester, citric and fatty acid esters of glycerol, fatty acid glyceride, glyceryl monolinoleate, glycerol monostearate, polysorbate, sorbitan fatty acid ester, polyoxyethylene fatty acid ester, Span, alginate, sodium oleate, and caseinate;
the stabilizer includes a terminal group provided by the PEG unit, and the terminal group is OH or methoxy;
the vitamin lipid polymer derivative is one or more selected from d-α-tocopheryl polyethylene glycol 200 succinate, d-α-tocopheryl polyethylene glycol 400 succinate, d-α-tocopheryl polyethylene glycol 1000 succinate, d-α-tocopheryl polyethylene glycol 1500 succinate, d-α-tocopheryl polyethylene glycol 2000 succinate, and d-α-tocopheryl polyethylene glycol 4000 succinate;
the phospholipid polymer derivative is selected from distearoylphosphatidylethanolamine-polyethylene glycol 2000, distearoylphosphatidylethanolamine-polyethylene glycol 5000, dipalmitoylphosphatidylethanolamine-methoxypolyethylene glycol 2000, dipalmitoylphosphatidylethanolamine-methoxypolyethylene glycol 5000, soybean phosphatidylethanolamine-polyethylene glycol monomethyl ether 2000, 1,2-di-myristoyl-rac-glycero-3-methoxypolyethylene glycol 2000, dilauroylphosphatidylethanolamine-polyethylene glycol 2000, and dioleoylphosphatidylethanolamine-polyethylene glycol;
the fatty acid ester polymer derivative is one or more selected from polyethylene glycol 400 oleate, polyethylene glycol 600 oleate, polyethylene glycol 4000 oleate, polyethylene glycol 6000 oleate, polyethylene glycol 400 dioleate, polyethylene glycol 600 dioleate, polyethylene glycol 200 laurate, polyethylene glycol 200 dilaurate, polyethylene glycol 400 laurate, polyethylene glycol 400 dilaurate, polyethylene glycol 400 stearate, and polyethylene glycol 400 distearate;
the polyoxyethylene-polyoxypropylene ether block copolymer is one or more selected from Pluronic L65 and Pluronic F68;
in the first auxiliary material, the amino acid with the side chain having the negatively charged group is one or more selected from aspartic acid, glutamic acid, and taurine;
in the first auxiliary material, the amino acid derivative with the side chain having the negatively charged group is one or more selected from phosphatidylserine, dihexadecyl-glutamate-glutamine, dihexadecyl-glutamate-glutamic acid, dihexadecyl-glutamate-asparagine;
in the first auxiliary material, the small peptide with the side chain having the negatively charged group is glutathione;
in the second auxiliary material, the antioxidant is one or more selected from vitamin E, α-tocopherol, β-tocopherol, γ-tocopherol, mixed tocopherols, α-tocopheryl acetate, β-tocopheryl acetate, γ-tocopheryl acetate, mixed tocopheryl acetates, ascorbic acid, ascorbyl palmitate, ascorbyl stearate, ascorbyl myristate, sodium ascorbate, butylated hydroxyanisole, dibutylated hydroxytoluene, propylgallate, and tert-butylhydroquinone;
in the second auxiliary material, the base oil is one or more selected from soybean oil, olive oil, jojoba oil, sweet almond oil, grapeseed oil, corn oil, walnut oil, sea buckthorn oil, olive oil, coix seed oil, grapeseed oil, ginger oil, coconut oil, camellia oil, rose oil, peppermint oil, lemon oil, and medium-chain triglycerides.

In some embodiments, the EPA-EE nano-lipid composition satisfies any one or any combination of the following features:
the first emulsifier is one or more selected from soybean phospholipid S75, soybean phospholipid S100, sunflower seed phospholipid H100, and polyene phosphatidylcholine;
the second emulsifier is one or more selected from egg yolk lecithin E80, polysorbate 80, and sorbitan oleate 80;
the stabilizer is one or more selected from TPGS, DSPE-PEG, and S40; optionally, the stabilizer is a combination of TPGS and S40, and further optionally, a mixture of TPGS and S40 in equal masses;
the first auxiliary material is one or more selected from phosphatidylserine, sodium glutamate, and taurine; optionally, the first auxiliary material is a combination of taurine and sodium glutamate, and further optionally, a mixture of taurine and sodium glutamate in equal masses;
the second auxiliary material includes an antioxidant and a base oil; optionally, the second auxiliary material is a combination of the antioxidant and the base oil; further optionally, the antioxidant is α-tocopherol, and the base oil is corn oil, olive oil, a medium-chain triglyceride, or a combination thereof; further optionally, the base oil is a combination of corn oil and olive oil; yet further optionally, the base oil is a mixture of corn oil and olive oil in equal masses.

In some embodiments, the EPA-EE nano-lipid composition includes the following components in parts by mass: 50 to 500 parts by mass of the EPA-EE raw material, 10 to 100 parts by mass of the first emulsifier, 0 to 100 parts by mass of the second emulsifier, 0 to 1.2 parts by mass of the α-tocopherol, 0 to 60 parts by mass of the base oil, and water.

Optionally, one or more of the following features is included:
the total weight of the EPA-EE nano-lipid composition is 900 to 1100 parts by mass, further optionally 1000 parts by mass;
the EPA-EE raw material takes 100 to 400 parts by mass, further optionally 100 to 300 parts by mass;
the first emulsifier takes 10 to 100 parts by mass, further optionally 10 to 50 parts by mass, yet further optionally 20 to 50 parts by mass;
the first emulsifier includes one or more selected from soybean phospholipid, sunflower seed phospholipid, and polyene phosphatidylcholine, further optionally the first emulsifier is soybean phospholipid, sunflower seed phospholipid, polyene phosphatidylcholine, or a combination thereof;
the second emulsifier takes 10 to 100 parts by mass, further optionally 10 to 50 parts by mass, yet further optionally 20 to 50 parts by mass;
the second emulsifier includes one or more selected from egg yolk lecithin, polysorbate, and sorbitan oleate 80, further optionally the second emulsifier is egg yolk lecithin, polysorbate, sorbitan oleate 80, or a combination thereof;
the α-tocopherol takes 0.1 to 1 part by mass, further optionally 0.2 to 1 part by mass, yet further optionally 0.4 to 1 part by mass, yet further optionally 0.5 to 1 part by mass;
the base oil takes 30 to 50 parts by mass; and
the base oil includes at least one of corn oil or olive oil, optionally the base oil is any one or a combination of corn oil and olive oil.

In some embodiments, the EPA-EE nano-lipid composition is any one of the following compositions each in 1000 parts by mass:
[Composition 1-1]: 100 parts by mass of EPA-EE 60, 10 parts by mass of soybean phospholipid Lipoid S75, and the balance of water;
[Composition 1-2]: 100 parts by mass of EPA-EE 60, 10 parts by mass of soybean phospholipid Lipoid S100, and the balance of water;
[Composition 1-3]: 100 parts by mass of EPA-EE 60, 10 parts by mass of sunflower seed phospholipid Lipoid H100, and the balance of water;
[Composition 1-4]: 100 parts by mass of EPA-EE 60, 10 parts by mass of polyene phosphatidylcholine, and the balance of water;
[Composition 1-5]: 100 parts by mass of EPA-EE 60, 10 parts by mass of soybean phospholipid Lipoid S75, and water;
[Composition 1-6]: 200 parts by mass of EPA-EE 60, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and the balance of water;
[Compositions 1-7]: 200 parts by mass of EPA-EE 60, 50 parts by mass of soybean phospholipid Lipoid S100, 10 parts by mass of polysorbate 80, 0.5 parts by mass of α-tocopherol, 30 parts by mass of corn oil, and the balance of water;
[Compositions 1-8]: 200 parts by mass of EPA-EE 60, 50 parts by mass of sunflower seed phospholipid Lipoid H100, 10 parts by mass of polysorbate 80, 0.5 parts by mass of α-tocopherol, 30 parts by mass of corn oil, and the balance of water;
[Compositions 1-9]: 200 parts by mass of EPA-EE 60, 50 parts by mass of polyene phosphatidylcholine, 10 parts by mass of sorbitan oleate 80, 0.5 parts by mass of α-tocopherol, 30 parts by mass of olive oil, and the balance of water;
[Compositions 1-10]: 200 parts by mass of EPA-EE 60, 50 parts by mass of soybean phospholipid Lipoid S75, 10 parts by mass of sorbitan oleate 80, 0.5 parts by mass of α-tocopherol, 30 parts by mass of olive oil, and the balance of water;
[Compositions 1-11]: 300 parts by mass of EPA-EE 60, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and the balance of water;
[Compositions 1-12]: 300 parts by mass of EPA-EE 60, 100 parts by mass of soybean phospholipid Lipoid S100, 20 parts by mass of polysorbate 80, 1 part by mass of α-tocopherol, 50 parts by mass of corn oil, and the balance of water;
[Compositions 1-13]: 300 parts by mass of EPA-EE 60, 100 parts by mass of sunflower seed phospholipid Lipoid H100, 20 parts by mass of polysorbate 80, 1 part by mass of α-tocopherol, 50 parts by mass of corn oil, and the balance of water;
[Compositions 1-14]: 300 parts by mass of EPA-EE 60, 100 parts by mass of polyene phosphatidylcholine, 20 parts by mass of sorbitan oleate 80, 1 part by mass of α-tocopherol, 50 parts by mass of olive oil, and the balance of water;
[Compositions 1-15]: 300 parts by mass of EPA-EE 60, 100 parts by mass of soybean phospholipid Lipoid S75, 20 parts by mass of sorbitan oleate 80, 1 part by mass of α-tocopherol, 50 parts by mass of olive oil, and the balance of water;
[Composition 2-1]: 100 parts by mass of EPA-EE 80, 10 parts by mass of soybean phospholipid Lipoid S75, and the balance of water;
[Composition 2-2]: 100 parts by mass of EPA-EE 80, 10 parts by mass of soybean phospholipid Lipoid S100, and the balance of water;
[Composition 2-3]: 100 parts by mass of EPA-EE 80, 10 parts by mass of sunflower seed phospholipid Lipoid H100, and the balance of water;
[Composition 2-4]: 100 parts by mass of EPA-EE 80, 10 parts by mass of polyene phosphatidylcholine, and the balance of water;
[Composition 2-5]: 100 parts by mass of EPA-EE 80, 10 parts by mass of soybean phospholipid Lipoid S75, and water;
[Composition 2-6]: 200 parts by mass of EPA-EE 80, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and the balance of water;
[Compositions 2-7]: 200 parts by mass of EPA-EE 80, 50 parts by mass of soybean phospholipid Lipoid S100, 10 parts by mass of polysorbate 80, 0.5 parts by mass of α-tocopherol, 30 parts by mass of corn oil, and the balance of water;
[Compositions 2-8]: 200 parts by mass of EPA-EE 80, 50 parts by mass of sunflower seed phospholipid Lipoid H100, 10 parts by mass of polysorbate 80, 0.5 parts by mass of α-tocopherol, 30 parts by mass of corn oil, and the balance of water;
[Compositions 2-9]: 200 parts by mass of EPA-EE 80, 50 parts by mass of polyene phosphatidylcholine, 10 parts by mass of sorbitan oleate 80, 0.5 parts by mass of α-tocopherol, 30 parts by mass of olive oil, and the balance of water;
[Compositions 2-10]: 200 parts by mass of EPA-EE 80, 50 parts by mass of soybean phospholipid Lipoid S75, 10 parts by mass of sorbitan oleate 80, 0.5 parts by mass of α-tocopherol, 30 parts by mass of olive oil, and the balance of water;
[Compositions 2-11]: 300 parts by mass of EPA-EE 80, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and the balance of water;
[Compositions 2-12]: 300 parts by mass of EPA-EE 80, 100 parts by mass of soybean phospholipid Lipoid S100, 20 parts by mass of polysorbate 80, 1 part by mass of α-tocopherol, 50 parts by mass of corn oil, and the balance of water;
[Composition 2-13]: 300 parts by mass of EPA-EE 80, 100 parts by mass of sunflower seed phospholipid Lipoid H100, 20 parts by mass of polysorbate 80, 1 part by mass of α-tocopherol, 50 parts by mass of corn oil, and the balance of water;
[Composition 2-14]: 300 parts by mass of EPA-EE 80, 100 parts by mass of polyene phosphatidylcholine, 20 parts by mass of sorbitan oleate 80, 1 part by mass of α-tocopherol, 50 parts by mass of olive oil, and the balance of water;
[Composition 2-15]: 300 parts by mass of EPA-EE 80, 100 parts by mass of soybean phospholipid Lipoid S75, 20 parts by mass of sorbitan oleate 80, 1 part by mass of α-tocopherol, 50 parts by mass of olive oil, and the balance of water;
[Composition 3-1]: 100 parts by mass of EPA-EE 97, 10 parts by mass of soybean phospholipid Lipoid S75, and the balance of water;
[Composition 3-2]: 100 parts by mass of EPA-EE 97, 10 parts by mass of soybean phospholipid Lipoid S100, and the balance of water;
[Composition 3-3]: 100 parts by mass of EPA-EE 97, 10 parts by mass of sunflower seed phospholipid Lipoid H100, and the balance of water;
[Composition 3-4]: 100 parts by mass of EPA-EE 97, 10 parts by mass of polyene phosphatidylcholine, and the balance of water;
[Composition 3-5]: 100 parts by mass of EPA-EE 97, 10 parts by mass of soybean phospholipid Lipoid S75, and water;
[Composition 3-6]: 200 parts by mass of EPA-EE 97, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and the balance of water;
[Compositions 3-7]: 200 parts by mass of EPA-EE 97, 50 parts by mass of soybean phospholipid Lipoid S100, 10 parts by mass of polysorbate 80, 0.5 parts by mass of α-tocopherol, 30 parts by mass of corn oil, and the balance of water;
[Compositions 3-8]: 200 parts by mass of EPA-EE 97, 50 parts by mass of sunflower seed phospholipid Lipoid H100, 10 parts by mass of polysorbate 80, 0.5 parts by mass of α-tocopherol, 30 parts by mass of corn oil, and the balance of water;
[Compositions 3-9]: 200 parts by mass of EPA-EE 97, 50 parts by mass of polyene phosphatidylcholine, 10 parts by mass of sorbitan oleate 80, 0.5 parts by mass of α-tocopherol, 30 parts by mass of olive oil, and the balance of water;
[Compositions 3-10]: 200 parts by mass of EPA-EE 97, 50 parts by mass of soybean phospholipid Lipoid S75, 10 parts by mass of sorbitan oleate 80, 0.5 parts by mass of α-tocopherol, 30 parts by mass of olive oil, and the balance of water;
[Compositions 3-11]: 300 parts by mass of EPA-EE 97, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and the balance of water;
[Composition 3-12]: 300 parts by mass of EPA-EE 97, 100 parts by mass of soybean phospholipid Lipoid S100, 20 parts by mass of polysorbate 80, 1 part by mass of α-tocopherol, 50 parts by mass of corn oil, and the balance of water;
[Composition 3-13]: 300 parts by mass of EPA-EE 97, 100 parts by mass of sunflower seed phospholipid Lipoid H100, 20 parts by mass of polysorbate 80, 1 part by mass of α-tocopherol, 50 parts by mass of corn oil, and the balance of water;
[Composition 3-14]: 300 parts by mass of EPA-EE 97, 100 parts by mass of polyene phosphatidylcholine, 20 parts by mass of sorbitan oleate 80, 1 part by mass of α-tocopherol, 50 parts by mass of olive oil, and the balance of water;
[Compositions 3-15]: 300 parts by mass of EPA-EE 97, 100 parts by mass of soybean phospholipid Lipoid S75, 20 parts by mass of sorbitan oleate 80, 1 part by mass of α-tocopherol, 50 parts by mass of olive oil, and the balance of water.

Examples of the EPA-EE nano-lipid composition include, but are not limited to, those enumerated in Example 1, wherein 1 g may be considered as 1 part by mass.

In some embodiments, the EPA-EE nano-lipid composition includes 50 to 500 parts by mass of the EPA-EE raw material (further can be 100 to 400 parts by mass, yet further can be 100 to 300 parts by mass), 10 to 100 parts by mass of the first emulsifier (such as 10, 20, 30, 40, 50 parts by mass, which can be selected from soybean phospholipids such as Lipoid S75 and Lipoid S100, sunflower seed phospholipid, polyene phosphatidylcholine, etc.), 0 to 100 parts by mass of the second emulsifier (such as 0, 10, 20, 30, 40, 50 parts by mass, which can be selected from egg yolk lecithin such as E80, polysorbate such as polysorbate 80, sorbitan oleate, etc.), 0 to 1.2 parts by mass of α-tocopherol (which further can be 0.1 to 1 part by mass, yet further can be 0.5 to 1 part by mass), 0 to 60 parts by mass of the base oil (which further can be 30 to 50 parts by mass, and can be selected from corn oil and olive oil), and water (in an appropriate amount). Further, the total weight of the EPA-EE nano-lipid composition can be 900 to 1100 parts by mass (optionally can be 1000 parts by mass). The types, formats, models, and amounts of individual components can be further referred to Example 1.1.

In some embodiments of the present application, the EPA-EE nano-lipid composition includes the following components: 50 to 500 parts by mass of the EPA-EE raw material, 10 to 100 parts by mass of the first emulsifier, 0 to 60 parts by mass of the stabilizer, 0 to 50 parts by mass of the first auxiliary material, 20 to 40 parts by mass of the second auxiliary material, and water.

Optionally, the EPA-EE nano-lipid composition further includes one or more of the following features:
the total weight of the EPA-EE nano-lipid composition is 900 to 1100 parts by mass, further optionally 1000 parts by mass;
the EPA-EE raw material takes 100 to 400 parts by mass, further optionally 100 to 200 parts by mass;
the first emulsifier takes 10 to 50 parts by mass;
the first emulsifier includes soybean phospholipid, further optionally is soybean phospholipid;
the stabilizer takes 10 to 50 parts by mass, further optionally 10 to 20 parts by mass;
the stabilizer includes one or more selected from TPGS, DSPE-PEG, and S40, further optionally TPGS, DSPE-PEG, S40, or any combination thereof;
the first auxiliary material takes 10 to 30 parts by mass, further optionally 10 to 20 parts by mass;
the first auxiliary material includes one or more selected from phosphatidylserine, sodium glutamate, and taurine; further optionally, the first auxiliary material is phosphatidylserine, sodium glutamate, taurine, or any combination thereof;
the second auxiliary material is a combination of an antioxidant and a base oil; optionally, the antioxidant is α-tocopherol, and the base oil is corn oil, olive oil, medium-chain triglyceride, or a combination thereof; further optionally, the base oil is a combination of corn oil and olive oil, or the base oil is olive oil or medium-chain triglyceride; further, the base oil is a mixture of corn oil and olive oil in equal masses; further optionally, the second auxiliary material consists of 1 to 2 parts by mass of the antioxidant and 25 to 35 parts by mass of the base oil.

In some embodiments of the present application, the EPA-EE nano-lipid composition in 1000 parts by mass is a combination of any one of the following compositions and the second auxiliary material, wherein the second auxiliary material is a combination of 1 part by mass of α-tocopherol and 30 parts by mass of olive oil, or the second auxiliary material is a combination of 2 parts by mass of α-tocopherol and 30 parts by mass of a medium-chain triglyceride:
[A1]: 100 parts by mass of EPA-EE 60, 10 parts by mass of soybean phospholipid Lipoid S75, 10 parts by mass of TPGS, and the balance of water;
[A2]: 100 parts by mass of EPA-EE 60, 10 parts by mass of DSPE-PEG, 10 parts by mass of sodium glutamate, and the balance of water;
[A3]: 100 parts by mass of EPA-EE 60, 10 parts by mass of S40, 10 parts by mass of phosphatidylserine, and the balance of water;
[A4]: 100 parts by mass of EPA-EE 60, 10 parts by mass of S40, 10 parts by mass of taurine, and the balance of water;
[A5]: 100 parts by mass of EPA-EE 60, 10 parts by mass of a mixture of TPGS and S40 in equal masses, 10 parts by mass of a mixture of taurine and sodium glutamate in equal masses, and the balance of water;
[A6]: 100 parts by mass of EPA-EE 60, 10 parts by mass of soybean phospholipid Lipoid S75, 10 parts by mass of TPGS, 10 parts by mass of phosphatidylserine, and the balance of water;
[A7]: 100 parts by mass of EPA-EE 60, 10 parts by mass of soybean phospholipid Lipoid S75, 10 parts by mass of DSPE-PEG, 10 parts by mass of sodium glutamate, and the balance of water;
[A8]: 100 parts by mass of EPA-EE 60, 10 parts by mass of soybean phospholipid Lipoid S75, 10 parts by mass of S40, 10 parts by mass of phosphatidylserine, and the balance of water;
[A9]: 100 parts by mass of EPA-EE 60, 10 parts by mass of soybean phospholipid Lipoid S75, 10 parts by mass of S40, 10 parts by mass of taurine, and the balance of water;
[A10]: 100 parts by mass of EPA-EE 60, 10 parts by mass of soybean phospholipid Lipoid S75, 10 parts by mass of a mixture of TPGS and S40 in equal masses, 10 parts by mass of a mixture of taurine and sodium glutamate in equal masses, and the balance of water;
[A11]: 200 parts by mass of EPA-EE 60, 100 parts by mass of soybean phospholipid Lipoid S75, 20 parts by mass of TPGS, 20 parts by mass of phosphatidylserine, and the balance of water;
[A12]: 200 parts by mass of EPA-EE 60, 100 parts by mass of soybean phospholipid Lipoid S75, 20 parts by mass of DSPE-PEG, 20 parts by mass of sodium glutamate, and the balance of water;
[A13]: 200 parts by mass of EPA-EE 60, 100 parts by mass of soybean phospholipid Lipoid S75, 20 parts by mass of S40, 20 parts by mass of phosphatidylserine, and the balance of water;
[A14]: 200 parts by mass of EPA-EE 60, 100 parts by mass of soybean phospholipid Lipoid S75, 20 parts by mass of S40, 20 parts by mass of taurine, and the balance of water;
[A15]: 200 parts by mass of EPA-EE 60, 100 parts by mass of soybean phospholipid Lipoid S75, 20 parts by mass of a mixture of TPGS and S40 in equal masses, 20 parts by mass of a mixture of taurine and sodium glutamate in equal masses, and the balance of water;
[B1]: 100 parts by mass of EPA-EE 80, 10 parts by mass of soybean phospholipid Lipoid S75, 10 parts by mass of TPGS, and the balance of water;
[B2]: 100 parts by mass of EPA-EE 80, 10 parts by mass of DSPE-PEG, 10 parts by mass of sodium glutamate, and the balance of water;
[B3]: 100 parts by mass of EPA-EE 80, 10 parts by mass of S40, 10 parts by mass of phosphatidylserine, and the balance of water;
[B4]: 100 parts by mass of EPA-EE 80, 10 parts by mass of S40, 10 parts by mass of taurine, and the balance of water;
[B5]: 100 parts by mass of EPA-EE 80, 10 parts by mass of a mixture of TPGS and S40 in equal masses, 10 parts by mass of a mixture of taurine and sodium glutamate in equal masses, and the balance of water;
[B6]: 100 parts by mass of EPA-EE 80, 10 parts by mass of soybean phospholipid Lipoid S75, 10 parts by mass of TPGS, 10 parts by mass of phosphatidylserine, and the balance of water;
[B7]: 100 parts by mass of EPA-EE 80, 10 parts by mass of soybean phospholipid Lipoid S75, 10 parts by mass of DSPE-PEG, 10 parts by mass of sodium glutamate, and the balance of water;
[B8]: 100 parts by mass of EPA-EE 80, 10 parts by mass of soybean phospholipid Lipoid S75, 10 parts by mass of S40, 10 parts by mass of phosphatidylserine, and the balance of water;
[B9]: 100 parts by mass of EPA-EE 80, 10 parts by mass of soybean phospholipid Lipoid S75, 10 parts by mass of S40, 10 parts by mass of taurine, and the balance of water;
[B10]: 100 parts by mass of EPA-EE 80, 10 parts by mass of soybean phospholipid Lipoid S75, 10 parts by mass of a mixture of TPGS and S40 in equal masses, 10 parts by mass of a mixture of taurine and sodium glutamate in equal masses, and the balance of water;
[B11]: 200 parts by mass of EPA-EE 80, 100 parts by mass of soybean phospholipid Lipoid S75, 20 parts by mass of TPGS, 20 parts by mass of phosphatidylserine, and the balance of water;
[B12]: 200 parts by mass of EPA-EE 80, 100 parts by mass of soybean phospholipid Lipoid S75, 20 parts by mass of DSPE-PEG, 20 parts by mass of sodium glutamate, and the balance of water;
[B13]: 200 parts by mass of EPA-EE 80, 100 parts by mass of soybean phospholipid Lipoid S75, 20 parts by mass of S40, 20 parts by mass of phosphatidylserine, and the balance of water;
[B14]: 200 parts by mass of EPA-EE 80, 100 parts by mass of soybean phospholipid Lipoid S75, 20 parts by mass of S40, 20 parts by mass of taurine, and the balance of water;
[B15]: 200 parts by mass of EPA-EE 80, 100 parts by mass of soybean phospholipid Lipoid S75, 20 parts by mass of a mixture of TPGS and S40 in equal masses, 20 parts by mass of a mixture of taurine and sodium glutamate in equal masses, and the balance of water;
[C1]: 100 parts by mass of EPA-EE 97, 10 parts by mass of soybean phospholipid Lipoid S75, 10 parts by mass of TPGS, and the balance of water;
[C2]: 100 parts by mass of EPA-EE 97, 10 parts by mass of DSPE-PEG, 10 parts by mass of sodium glutamate, and the balance of water;
[C3]: 100 parts by mass of EPA-EE 97, 10 parts by mass of S40, 10 parts by mass of phosphatidylserine, and the balance of water;
[C4]: 100 parts by mass of EPA-EE 97, 10 parts by mass of S40, 10 parts by mass of taurine, and the balance of water;
[C5]: 100 parts by mass of EPA-EE 97, 10 parts by mass of a mixture of TPGS and S40 in equal masses, 10 parts by mass of a mixture of taurine and sodium glutamate in equal masses, and the balance of water;
[C6]: 100 parts by mass of EPA-EE 97, 10 parts by mass of soybean phospholipid Lipoid S75, 10 parts by mass of TPGS, 10 parts by mass of phosphatidylserine, and the balance of water;
[C7]: 100 parts by mass of EPA-EE 97, 10 parts by mass of soybean phospholipid Lipoid S75, 10 parts by mass of DSPE-PEG, 10 parts by mass of sodium glutamate, and the balance of water;
[C8]: 100 parts by mass of EPA-EE 97, 10 parts by mass of soybean phospholipid Lipoid S75, 10 parts by mass of S40, 10 parts by mass of phosphatidylserine, and the balance of water;
[C9]: 100 parts by mass of EPA-EE 97, 10 parts by mass of soybean phospholipid Lipoid S75, 10 parts by mass of S40, 10 parts by mass of taurine, and the balance of water;
[C10]: 100 parts by mass of EPA-EE 97, 10 parts by mass of soybean phospholipid Lipoid S75, 10 parts by mass of a mixture of TPGS and S40 in equal masses, 10 parts by mass of a mixture of taurine and sodium glutamate in equal masses, and the balance of water;
[C11]: 200 parts by mass of EPA-EE 97, 100 parts by mass of soybean phospholipid Lipoid S75, 20 parts by mass of TPGS, 20 parts by mass of phosphatidylserine, and the balance of water;
[C12]: 200 parts by mass of EPA-EE 97, 100 parts by mass of soybean phospholipid Lipoid S75, 20 parts by mass of DSPE-PEG, 20 parts by mass of sodium glutamate, and the balance of water;
[C13]: 200 parts by mass of EPA-EE 97, 100 parts by mass of soybean phospholipid Lipoid S75, 20 parts by mass of S40, 20 parts by mass of phosphatidylserine, and the balance of water;
[C14]: 200 parts by mass of EPA-EE 97, 100 parts by mass of soybean phospholipid Lipoid S75, 20 parts by mass of S40, 20 parts by mass of taurine, and the balance of water;
[C15]: 200 parts by mass of EPA-EE 97, 100 parts by mass of soybean phospholipid Lipoid S75, 20 parts by mass of a mixture of TPGS and S40 in equal masses, 20 parts by mass of a mixture of taurine and sodium glutamate in equal masses, and the balance of water.

In some embodiments of the present application, the EPA-EE nano-lipid composition includes 50 to 500 parts by mass of the EPA-EE raw material (further can be 100 to 400 parts by mass, yet further can be 100 to 200 parts by mass), 10 to 100 parts by mass of the first emulsifier (which further can be such as 10, 20, 30, 40, 50 parts by mass, and can be selected from soybean phospholipid such as Lipoid S75), 0 to 60 parts by mass of the stabilizer (which further can be 10 to 50 parts by mass, yet further can be 10 to 20 parts by mass, and can be selected from TPGS, DSPE-PEG, S40, etc.), 0 to 50 parts by mass of the first auxiliary material (which further can be 10 to 30 parts by mass, yet further can be 10 to 20 parts by mass, and can be selected from phosphatidylserine, sodium glutamate, taurine, etc.), and water (in an appropriate amount). Further, the total weight of the EPA-EE nano-lipid composition can be 900 to 1100 parts by mass (such as 1000 parts by mass). The types, formats/models, and amounts of individual components can be further referred to Example 1.2.

In the above embodiments, the types and amounts of the individual components can be referred to the above description, and can be independent from each other.

In the above embodiments, the individual components can be independent from each other. The “appropriate amount” of water is adapted for emulsification with a suitable particle size.

In the above embodiments, the total weight of the EPA-EE nano-lipid composition can be about 1000 parts by mass (reference can be made to Formulation Example 1).

In some embodiments of the present application, the EPA-EE nano-lipid composition is in form of submicron emulsion, and furthermore, with an average particle size less than or equal to 500 nm. In some embodiments of the present application, the EPA-EE nano-lipid composition is a highly dispersed drug carrier (nano-lipid carrier) with an average particle size ranging from 10 nm to 500 nm, which facilitates the absorption of EPA by intestinal epithelial cells, allowing EPA to enter the mesenteric capillaries and reach the body circulation, thereby improving the oral bioavailability and increasing the blood concentration of EPA. In some embodiments of the present application, the average particle size of the droplets in the submicron emulsion is less than 500 nm, and furthermore, the average particle size can be less than or equal to 300 nm (e.g., 100 nm to 300 nm), further can be less than or equal to 250 nm, yet further can be about 200 nm. In some non-limiting embodiments of the present application, the average particle size of the droplets in the submicron emulsion is about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 400 nm, about 450 nm, or about 500 nm.

In some embodiments of the present application, the droplet size of the submicron emulsion is ranged from 100 nm to 300 nm, which can effectively reduce the blood lipids and help in reducing atherosclerotic plaque formation.

For any of the above embodiments of the EPA-EE nano-lipid composition including the first auxiliary material, the first auxiliary material can be further omitted, as long as the following effects can be achieved: the effective EPA concentration in blood can be maintained for a long period, or the oral absorption and bioavailability of EPA can be improved.

### Second Aspect of the Present Application

According to a second aspect of the present application, an EPA-EE nano-lipid formulation is provided, including the EPA-EE nano-lipid composition described in the first aspect of the present application. The EPA-EE nano-lipid formulation is one of the formulations of the EPA-EE nano-lipid composition.

The EPA-EE nano-lipid formulation has a unique nano-lipid composition for carrying a high concentration of EPA-EE, so as to meet the dosage requirement of EPA for effective lipid-lowering and atherosclerosis treatment in vivo upon oral administration.

The lipid formulation can include highly dispersed nanoscale droplets (e.g., the droplets with an average particle size of 10 nm to 500 nm), which can facilitate the absorption of EPA by the intestinal epithelial cells, allowing EPA to enter the mesenteric capillaries and reach the body circulation, thereby improving the oral bioavailability and the EPA concentration in blood. In some non-limiting embodiments of the present application, the average particle size of the droplets in the EPA-EE nano-lipid formulation is about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 400 nm, about 450 nm, or about 500 nm.

In some embodiments, the EPA-EE nano-lipid formulation is an oral formulation.

In some embodiments of the present application, the EPA-EE nano-lipid formulation is an oral emulsion. In some embodiments of the present application, the average particle size of the droplets in the oral emulsion is less than 500 nm, and further can be less than or equal to 300 nm, yet further can be less than or equal to 250 nm, yet further can be about 200 nm. The non-limiting embodiments of the particle size of the droplets in the oral emulsion can refer to the above embodiments of the particle size in the lipid formulation, such as 100 nm to 300 nm.

In some embodiments of the present application, the EPA-EE nano-lipid formulation has an oil-in-water structure, in which the oil components such as the EPA-EE ester are in the oil phase.

The EPA-EE nano-lipid formulation can be obtained by using the preparation method in the third aspect of the present application.

In some embodiments of the present application, the EPA-EE nano-lipid formulation can achieve the following effect: after oral administration of 400 mg/kg of the formulation to a rat, the maximum blood drug concentration is reached within 2 hours and is greater than 550 μg/mL, and optionally greater than 700 μg/mL within 2 hours.

In some embodiments of the present application, the EPA-EE nano-lipid formulation can achieve the following effect: after oral administration of 400 mg/kg of the formulation to a rat, the EPA concentration in blood (including serum or plasma) is maintained above 200 μg/mL for over 2.5 h, and is maintained above 100 μg/mL for over 9 h. Further, the EPA concentration is maintained above 200 g/mL for over 3 h, and is maintained above 100 μg/mL for over 10 h.

### Third Aspect of the Present Application

According to a third aspect of the present application, a preparation method of an EPA-EE nano-lipid formulation is provided, which can be used to prepare the EPA-EE nano-lipid formulation in the second aspect of the present application.

The preparation method can be any one selected from emulsification, high-pressure homogenization, high-shear method, ultrasonic emulsification, microfluidization, etc.

In some embodiments of the present application, the preparation method includes the following steps:
S100, preparation of oil phase matrix (optionally under the protection of an inert gas): Oil phase components including the EPA-EE raw material are mixed uniformly under a heating condition (the heating temperature can be 50° C. to 70° C., for example, 50° C., 55° C., 60° C., 65° C., 70° C.) to prepare an oil phase matrix.
S200, preparation of aqueous phase matrix (optionally under the protection of an inert gas): Aqueous phase components are dissolved into an aqueous solvent until clear to prepare an aqueous phase matrix, or water is used as the aqueous phase matrix. Optionally, the aqueous phase can be pre-heated to a temperature (optionally the same or similar as the temperature for preparing the oil phase, such as 50° C. to 70° C., for example, 50° C., 55° C., 60° C., 65° C., 70° C.).
S300, preparation of primary emulsion: The oil phase matrix and the aqueous phase matrix are mixed together (optionally under a heating condition, further optionally, at 50° C. to 70° C., for example, 50° C., 55° C., 60° C., 65° C., 70° C.), shear-stirred and added with water to a predetermined volume to prepare an oil-in-water primary emulsion.
S400, preparation of submicron emulsion: The oil-in-water primary emulsion is subjected to high-pressure homogenization to form a submicron emulsion. Optionally, the average particle size of the submicron emulsion is less than or equal to 500 nm, further optionally less than or equal to 300 nm, or can be 100 nm to 300 nm.

The oil phase components refer to lipid-soluble components. The aqueous phase components refer to water-soluble components. The amphiphilic components can be as either the oil phase components or the aqueous phase components.

The primary emulsion and the submicron emulsion are non-limiting examples of the formulation of the EPA-EE nano-lipid composition of the present application.

In some embodiments of the present application, the prepared submicron emulsion is further subjected to filtration.

In some embodiments of the present application, the prepared submicron emulsion is further subjected to encapsulation.

In some embodiments of the present application, the prepared submicron emulsion is further subjected to sterilization.

In some embodiments of the present application, the prepared submicron emulsion is further subjected to packaging.

In some embodiments of the present application, the prepared submicron emulsion is further subjected to any one or more steps of filtration, encapsulation, and sterilization.

The above steps of filtration, encapsulation, sterilization, and packaging are each independently optional. Optionally, step S400 is followed by S500, post-processing (optionally under the protection of an inert gas): The submicron emulsion is subjected to filtration, encapsulation, and sterilization to obtain a sterilized EPA-EE nano-lipid formulation. The obtained formulation can be used as an oral emulsion.

In some embodiments, in the step of mixing the oil phase components including the EPA-EE raw material under the heating condition, the heating temperature is 50° C. to 70° C.

In some embodiments, in the step of mixing the oil phase matrix with the aqueous phase matrix, the mixing is performed at 50° C. to 70° C.

In some embodiments, a pH adjuster is added to the oil-in-water primary emulsion before subjecting the oil-in-water primary emulsion to high-pressure homogenization.

In some embodiments, the pressure of the high-pressure homogenization is 200 bar to 800 bar; further optionally, the high pressure homogenization is performed once or many times.

In some embodiments, the pH value of the EPA-EE nano-lipid formulation is 7 to 8.

Nitrogen gas can be used in the inert gas protection in the above preparation process.

The order of the above steps of the preparation method is not limited, unless otherwise specifically indicated. For example, the steps of S100 and S200 are not distinguished in order, but both are before S300. For another example, the order of encapsulation and sterilization is not restricted.

As used herein, “aqueous solvent” refers to a pharmaceutically acceptable solvent for the aqueous phase components, and the aqueous solvent can be water or a mixture of water and other solvents.

In some embodiments, the pH adjuster is mixed with water to obtain the aqueous solvent for subsequent preparation, and the pH adjuster is configured to adjust the pH value of the aqueous solvent. The pH value of the aqueous solvent is adjusted according to the preparation requirement for the final emulsion, according to which the suitable pH adjuster is selected. In some embodiments, the pH value of the final emulsion is 7 to 8.

In some embodiments, the pH adjuster is included in the raw materials for the preparation, and the pH value after the dispersion in S300 is adjusted. The pH value of the system is suitable to achieve stable and suitable particle size for medicinal use (in particular, for oral medicinal use) without affecting the performance of the drug activity. For example, the pH value can be adjusted to 7 to 8.

In some embodiments of the present application, the EPA-EE nano-lipid formulation can be prepared using a combination of a stator-rotor shearer and a high-pressure homogenizer or microfluidizer. The operating parameters can be adjusted according to the required particle size. In some embodiments, the rotation speed of the stator-rotor shearer is 7000 rpm to 10000 rpm, for example, 7000 rpm, 8000 rpm, 9000 rpm, 10000 rpm. The stirring time can be 3 min to 5 min, for example, 3 min, 4 min, 5 min. In some embodiments, the pressure of the high-pressure homogenization is 200 bar to 800 bar, for example, 200 bar, 300 bar, 400 bar, 500 bar, 600 bar, 700 bar, 800 bar. The homogenization can be performed once or many times, preferably many times, for example, 3 to 10 times, for another example, 2 times, 3 times, 4 times, 5 times, 6 times, 7 times, 8 times, 9 times, 10 times.

In some non-limiting embodiments of the present application, the preparation method includes the following steps: mixing EPA-EE with other lipid-soluble components to obtain a mixture as an oil phase matrix (also referred to as an oil phase), and preheating to a temperature such as 50° C. to 70° C.; adding the water soluble components of the EPA-EE nano-lipid composition to water, and preheating to a temperature (which can be the same or similar as the temperature for preparing the oil phase, for example, 50° C. to 70° C.) to obtain an aqueous solution as an aqueous phase matrix (also referred to as an aqueous phase); mixing the preheated oil phase matrix and the aqueous solution uniformly under stirring by using a stator-rotor shearer to obtain a primary emulsion, and treating the obtained primary emulsion with a high-pressure homogenizer to prepare a submicron emulsion with a certain particle size; subjecting or not subjecting the obtained submicron emulsion to filtration; filling the submicron emulsion into a bottle for oral administration and sealing the bottle under nitrogen protection, and sterilizing at an elevated temperature (optionally 100° C. to 121° C.) to obtain a sterilized EPA-EE nano-lipid formulation.

In some non-limiting embodiments of the present application, the preparation method includes the following steps:

S100: The oil phase components are mixed and stirred under the protection of an inert gas to form a homogeneous oil solution, which is heated in water bath to 50° C. to 70° C. to obtain a preheated oil phase; in some embodiments, the inert gas is nitrogen.

S200: The aqueous phase components of the composition are mixed under the protection of an inert gas, stirred until dissolved to form a homogeneous aqueous solution, which is heated in water bath to 50° C. to 70° C. to obtain a preheated aqueous phase; in some embodiments, the inert gas is nitrogen;

S300: The preheated aqueous phase is mixed with the oil phase, sheared or high-pressure homogenized to form an oil-in-water submicron emulsion (an unsterilized EPA-EE nano-lipid formulation).

Further, the obtained submicron emulsion can be heated in water bath to 50° C. to 70° C., then filtered, sterilized, and packaged under the protection of an inert gas to obtain a sterilized EPA-EE nano-lipid formulation.

In the case of specifying the types and the amounts of the raw materials, the person skilled in the art can implement the above preparation method in accordance with the above guidelines to obtain the EPA-EE nano-lipid formulation of the present application.

### Fourth Aspect of the Present Application

According to a fourth aspect of the present application, an application of the EPA-EE nano-lipid composition described in the first aspect of the present application, or the EPA-EE nano-lipid formulation described in the second aspect of the present application, or the EPA-EE nano-lipid formulation obtained by the preparation method described in the third aspect of the present application is provided. Further, the application includes an application in preparation of a drug for preventing and/or treating a disease, in particular a cardiovascular disease, and further includes an application in medical foods or health foods.

In the present application, unless otherwise specified, “drug for preventing and/or treating a cardiovascular disease” includes “drug for preventing a cardiovascular disease”, “drug for treating a cardiovascular disease”, and “drug for preventing and treating a cardiovascular disease”; “drug for preventing and treating a cardiovascular disease” refers to a drug capable of preventing and treating a cardiovascular disease.

In the present application, unless otherwise specified, “drug for preventing and/or treating a cardiovascular disease” refers to a drug capable of preventing cardiovascular disease, a drug capable of treating cardiovascular disease, or a drug capable of preventing and treating cardiovascular disease.

In some embodiments of the present application, an application of the EPA-EE nano-lipid composition in the first aspect of the present application, or the EPA-EE nano-lipid formulation in the second aspect of the present application, or the EPA-EE nano-lipid formulation obtained by the preparation method in the third aspect of the present application in preparation of a drug is provided. Further, the drug can be used for preventing and/or treating a fat accumulation related disease.

In some embodiments of the present application, the cardiovascular disease is atherosclerosis.

As demonstrated by the inventors' experiments, the EPA-EE nano-lipid formulation prepared in the present application contains a high concentration of EPA-EE, which enhances the oral absorption of EPA. Compared to directly ingesting fish oil, the peak time of EPA is advanced by 1 hour, and the bioavailability of EPA is also increased. With the stabilizer that maintains the EPA concentration in blood and the first auxiliary material that promotes the binding of EPA to lipoproteins, the EPA-EE nano-lipid formulation can reduce or delay the rapid clearance of EPA in the plasma. This prolongs the interaction time between EPA and lipid components in the plasma, and the content of EPA in lipoproteins is increased by EPA-EE actively binding to lipoproteins. Compared with ordinary fish oil or fish oil capsules, the EPA-EE nano-lipid formulation prepared in the present application has higher bioavailability, can fully exert blood lipid-lowering and atherosclerosis treating effects of EPA, thereby promoting the restoration of normal physiological state of the organism.

### Fifth Aspect of the Present Application

According to a fifth aspect of the present application, a method for preventing and/or treating a cardiovascular disease is provided, including administering to a patient in need thereof a therapeutically effective amount of the EPA-EE nano-lipid composition (in the first aspect) or the EPA-EE nano-lipid formulation (in the second or third aspect) as described in the present application. Further, the cardiovascular disease can be atherosclerosis.

As used herein, “therapeutically effective amount” refers to the amount of the EPA-EE nano-lipid (or the amount of EPA) of the present application that leads to a biological or medical response in an individual, e.g., the amount of the EPA-EE nano-lipid (or an amount of EPA) of the present application that leads to a physiologically and/or pharmacologically positive effect in an individual. The physiologically and/or pharmacologically positive effect includes, but is not limited to, reducing or inhibiting enzyme or protein activity or ameliorating symptoms, alleviating conditions, slowing or delaying disease progression, preventing disease, etc.

In the present application, unless otherwise specified, “physiologically and/or pharmacologically” refers to at least one of “physiologically”, “pharmacologically”, and “physiologically and pharmacologically”.

As used herein, “pharmaceutically acceptable” refers to any one or any combination of those agents, materials, compositions, and dosage forms that are suitable for administration to a patient within the bounds of reasonable medical judgment and that are commensurate with a reasonable “benefit/risk ratio”.

As used herein, “patient” refers to an animal, optionally a mammal, and further optionally a human. The term “mammal” refers primarily to a warm-blooded vertebrate mammal, including, but not limited to: a cat, a dog, a rabbit, a bear, a fox, a wolf, a monkey, a deer, a rat, a pig, a cow, a sheep, a horse, and human.

As used herein, unless otherwise specified, “administering” refers to administering the EPA-EE nano-lipid formulation of the present application.

In some embodiments of the present application, the subject is a rat, which is administered with the formulation at a dosage of 400 mg/kg, in single or multiple doses.

In some embodiments of the present application, the EPA-EE nano-lipid formulation of the present application is orally administered to a rat. The maximum blood drug concentration is achieved within 2 hours after the oral administration of 400 mg/kg to the rat, and the maximum blood drug concentration is greater than 550 μg/mL, in some embodiments, greater than 700 μg/mL.

In some embodiments of the present application, the EPA-EE nano-lipid formulation of the present application is orally administered to a rat. The EPA concentration in blood (including serum or plasma) after the oral administration of 400 mg/kg of the formulation to the rat is maintained above 200 g/mL for over 2.5 h, and maintained above 100 μg/mL for over 9 h. Further, the EPA concentration is maintained above 200 μg/mL for over 3 h, and maintained above 100 μg/mL for over 10 h.

### Sixth Aspect of the Present Application

According to a sixth aspect of the present application, a method for preventing and/or treating a cardiovascular disease is provided, including administering to a subject a therapeutically effective amount of the EPA-EE nano-lipid composition in the first aspect of the present application, or administering to a subject a therapeutically effective amount of the EPA-EE nano-lipid formulation in the second aspect of the present application, or administering to a subject a therapeutically effective amount of the EPA-EE nano-lipid formulation obtained by the preparation method in the third aspect of the present application.

In some conditions, the novel therapy can satisfy any one or any combination of the following features:
the cardiovascular disease is one or more events selected from the group consisting of hyperlipidemia, severe hypertriglyceridemia, extreme hypertriglyceridemia, atherosclerosis, occlusive atherosclerosis accompanied by ulceration and/or pain and coldness, carotid artery plaque, myocardial infarction, ischemic heart attack, ischemic attack, acute angina pectoris, hospitalization for acute angina pectoris, stroke, and hospitalization for a cardiovascular event; optionally, the cardiovascular disease is atherosclerosis;
the administration is by oral;
the subject is a patient suffering from the cardiovascular disease;
the subject is a mammal;
optionally, the subject is a human, or alternatively, the subject is a rat, and further optionally, the dosage of administration is 400 mg/kg, and further optionally, the administration is performed once or multiple times;
alternatively, the subject is a beagle, and further optionally, the dosage of administration is 120 mg/kg, and further optionally, the administration is performed once or multiple times.

In some embodiments, the nano-lipid formulation is administered to a male rat at a dosage of 400 mg/kg in terms of EPA-EE.

Further, the method can satisfy one or more of the following features:
from 0 to 24 hours after the administration, the area under the curve of the blood EPA concentration is greater than or equal to 2500 μg/mL, or the maximum blood EPA concentration is greater than or equal to 700 μg/mL;
from 0 to 24 hours after the administration, the area under the curve of the blood EPA concentration is greater than or equal to 3000 μg/mL, or the maximum blood EPA concentration is greater than or equal to 1400 μg/mL;
from 0 to 24 hours after the administration, the area under the curve of the blood EPA concentration is greater than or equal to 4000 μg/mL;
the blood EPA concentration is maintained above 200 μg/mL over 3 hours, and maintained above 100 g/mL over 6 hours; optionally, the blood EPA concentration is maintained above 100 g/mL over 10 hours.

In some embodiments, the nano-lipid formulation is administered to a male beagle at a dosage of 120 mg/kg in terms of EPA-EE.

Further, the method can satisfy one or more of the following features:
from 0 to 48 hours after the administration, the area under the curve of the blood EPA concentration is greater than or equal to 1500 μg/mL, or the maximum blood EPA concentration is greater than or equal to 100 μg/mL;
from 0 to 48 hours after the administration, the area under the curve of the blood EPA concentration is greater than or equal to 1900 μg/mL;
the blood EPA concentration is maintained above 60 μg/mL over 4 hours.

The embodiments of the present application will be described in detail below in combination with the examples. It should be understood that these examples are only for illustrating the present application and not for limiting the scope of the present application, and that these examples are only for exemplifying the present application to illustrate the composition of the formulation, the preparation method, and the functions and effects of the present application, and they should not be construed as limiting the scope of the present application in any form. Improvements and modifications can also be made without departing from the technical principles of the present application, which shall also be considered as within the scope of protection of the present application. For the experimental methods in the following examples in which detailed conditions are not specified, reference is given in preference to the guidelines given in the present application, and may also be made in accordance with the experimental manuals or conventional conditions in the art, and may also be made in accordance with the conditions recommended by the manufacturer, or with reference to the experimental methods known in the art.

In the following examples, when the measurement parameters of the raw material components are involved, there may be minor deviations within the weighing accuracy, unless otherwise specified. When temperature and time parameters are involved, acceptable deviations due to instrumental testing accuracy or operational precision are allowed.

In the following examples, unless otherwise specified, “room temperature” refers to 20° C. to 30° C.

In the following examples, unless otherwise specified, TPGS refers to vitamin E polyethylene glycol succinate.

Information on EPA-EE, phospholipids, and other raw materials used in the following examples and comparative examples is shown in Table 1.

TABLE 1
EPA-EE, phospholipids, and other raw materials used in the examples
and comparative examples of the present application

| Reagent type | Format and model | Manufacturer | Description |
|---|---|---|---|
| EPA-EE 60 | 6015 EE EPA60% + | KinOmega Corp. | 60 g of EPA-EE per 100 g |
| | DHA12% | | |
| EPA-EE 70 | KinOmega 7010 EE | KinOmega Corp. | 70 g of EPA-EE per 100 g |
| | EPA70% + DHA8% | | |
| EPA-EE 80 | K85EE | BASF Corp. | 80 g of EPA-EE per 100 g |
| | Omega-3-acid-EE | | |
| | (EPA EE | | |
| | 86227-47-6) | | |
| EPA-EE 97 | Maxomega EPA 97 | BASF Corp. | 97 g of EPA-EE per 100 g |
| | EE | | |
| Ordinary fish oil | Content of fish oil | BY-HEALTH Co., | 18.3% of EPA |
| | soft capsules | Ltd. | |
| EPA-EE capsules | Marketed product | Amarin | EPA-EE capsules with an EPA purity |
| | Vascepa | Corporation, US | of more than 97% |
| First emulsifier | Soybean | Lipoid, Germany | 70% of phosphatidylcholine, with the |
| | phospholipid Lipoid | | iodine value of 85 to 95 |
| | S75 | | |
| First emulsifier | Soybean | Lipoid, Germany | 94% of phosphatidylcholine, with the |
| | phospholipid Lipoid | | iodine value of 85 to 102 |
| | S100 | | |
| First emulsifier | Sunflower seed | Lipoid, Germany | 94% of phosphatidylcholine, with the |
| | phospholipid Lipoid | | iodine value of 95 to 113 |
| | H100 | | |
| First emulsifier | Polyene | Cargill, US | With the iodine value of 94 to 97 |
| | phosphatidylcholine | | |
| Second | Egg yolk lecithin | Lipoid, Germany | With the iodine value of 60 to 70 |
| emulsifier | E80 | | |
| Second | Polysorbate 80 | Shandong | Light yellow liquid |
| emulsifier | (Tween 80) | Ruisheng | |
| | | Pharmaceutical | |
| | | Excipient Co., Ltd. | |
| Second | Sorbitan oleate | Zhejiang Longyou | Light yellow liquid |
| emulsifier | (Span 80) | Juxing Medical | |
| | | Technology Co., | |
| | | LTD | |
| Second | Glyceryl | GATTEFOSSE | Light yellow liquid or yellow liquid |
| emulsifier | monolinoleate | SAS | |
| Second | Sodium oleate | Xi'an Libon | White powder |
| emulsifier | | Pharmaceutical | |
| | | Co., LTD | |
| Stabilizer | Vitamin E | Nanjing Weir | White powder, with the molecular |
| | polyethylene glycol | Pharmaceutical | weight of the polyethylene glycol unit |
| | succinate (TPGS) | Group Co., LTD | of 400 to 2000 |
| | | PMC Isochem | White solid, with the molecular |
| | | | weight of the polyethylene glycol unit |
| | | | of 1000 |
| Stabilizer | DSPE-PEG | Avito (Shanghai) | White powder, with the molecular |
| | | Pharmaceutical | weight of the polyethylene glycol unit |
| | | Technology Co., | of 2000 |
| | | LTD | |
| Stabilizer | Polyoxyethylene | Jaffa Lion | White solid |
| | stearate S40 | (Shanghai) | |
| | | Trading Co., LTD | |
| Stabilizer | Pluronic F68 | BASF SE | White solid |
| First auxiliary | Taurine | Hunan Xinlufang | White powder |
| material | | Pharmaceutical | |
| | | Co., LTD | |
| First auxiliary | Sodium glutamate | Hunan Xinlufang | White particles |
| material | | Pharmaceutical | |
| | | Co., LTD | |
| First auxiliary | Phosphatidylserine | Avito (Shanghai) | Light yellow solid |
| material | | Pharmaceutical | |
| | | Technology Co., | |
| | | LTD | |
| Antioxidant | α-tocopherol | Braun Medical | Light yellow, viscous, and oily liquid |
| | | (Shanghai) | |
| | | International Trade | |
| | | Co., LTD | |
| Base oil | Corn oil | Shandong | Light yellow oily liquid |
| | | Ruisheng | |
| | | Pharmaceutical | |
| | | Excipient Co., Ltd. | |
| Base oil | Olive oil | Luofu | Light yellow oily liquid |
| | | Pharmaceutical | |
| | | Technology | |
| | | (Shanghai) Co., | |
| | | LTD | |
| Base oil | Medium-chain | Liaoning Xinxing | Light yellow oily liquid |
| | triglyceride | Pharmaceutical | |
| | | Co., LTD | |

In the following examples, EPA-EE refers to eicosapentaenoic acid ethyl ester, and PC refers to phosphatidylcholine. In the following examples, the particle size was measured using the Zetasizer Nano ZS 90 (Malvern) laser particle size analyzer.

### I. FORMULATION EXAMPLES

### Example 1.1. Preparation of EPA-EE Nano-Lipid Formulations Containing EPA-EE and an Emulsifier

### 1.1.1. Raw Materials

Eicosapentaenoic acid-EE nano-lipid formulations (also referred to as EPA-EE nano-lipid formulations) were prepared using the raw materials in the amounts shown in Table 2. EPA-EE 60 was from KinOmega Corp., with the model of 6015 EE EPA 60%+DHA 12%, containing 60 g of EPA-EE per 100 g of EPA-EE 60. EPA-EE 80 was from BASF Corp. with the model of K85EE Omega-3-acid-EE (EPA EE 86227-47-6), containing 80 g of EPA-EE per 100 g of EPA-EE 80. EPA-EE 97 was from BASF Corp. with the model of Maxomega EPA 97 EE, containing about 97 g of EPA-EE per 100 g of EPA-EE 97. Information on the raw materials also refers to Table 1.

TABLE 2
Compositions and particle sizes of different EPA-EE nano-lipid formulations

| | | First | Second | Anti- | Base | | Particle |
|---|---|---|---|---|---|---|---|
| Formulation | EPA-EE | emulsifier | emulsifier | oxidant | oil | | size |
| No. | 60 (g) | (g) | (g) | (g) | (g) | Water | (nm) |
| 1-1 to 1-5 | 100 | 10 | 0 | 0 | 0 | balance | 150~260 |
| | | | | | | to 1 kg | |
| 1-6 to 1-10 | 200 | 50 | 10 | 0.5 | 30 | balance | 115~250 |
| | | | | | | to 1 kg | |
| 1-11 to 1-15 | 300 | 100 | 20 | 1 | 50 | balance | 175~280 |
| | | | | | | to 1 kg | |
| | | First | Second | Anti- | Base | | Particle |
| Formulation | EPA-EE | emulsifier | emulsifier | oxidant | oil | | size |
| No. | 80 (g) | (g) | (g) | (g) | (g) | Water | (nm) |
| 2-1 to 2-5 | 100 | 10 | 0 | 0 | 0 | balance | 150~260 |
| | | | | | | to 1 kg | |
| 2-6 to 2-10 | 200 | 50 | 10 | 0.5 | 30 | balance | 180~290 |
| | | | | | | to 1 kg | |
| 2-11 to 2-15 | 300 | 100 | 20 | 1 | 50 | balance | 190~260 |
| | | | | | | to 1 kg | |
| | | First | Second | Anti- | Base | | Particle |
| Formulation | EPA-EE | emulsifier | emulsifier | oxidant | oil | | size |
| No. | 97 (g) | (g) | (g) | (g) | (g) | Water | (nm) |
| 3-1 to 3-5 | 100 | 10 | 0 | 0 | 0 | balance | 150~260 |
| | | | | | | to 1 kg | |
| 3-6 to 3-10 | 200 | 50 | 10 | 0.5 | 30 | balance | 150~250 |
| | | | | | | to 1 kg | |
| 3-11 to 3-15 | 300 | 100 | 100 | 1 | 50 | balance | 170~260 |
| | | | | | | to 1 kg | |

In formulations 1-1, 1-6, and 1-11, formulations 2-1, 2-6, and 2-11, and formulations 3-1, 3-6, and 3-11, the first emulsifier was soybean phospholipid Lipoid S75, the second emulsifier was egg yolk lecithin E80, the antioxidant was α-tocopherol, and the base oil was a mixture of corn oil and olive oil in equal masses.

In formulations 1-2, 1-7, and 1-12, formulations 2-2, 2-7, and 2-12, and formulations 3-2, 3-7, and 3-12, the first emulsifier was soybean phospholipid Lipoid S100, the second emulsifier was polysorbate 80, the antioxidant was α-tocopherol, and the base oil was corn oil.

In formulations 1-3, 1-8, and 1-13, formulations 2-3, 2-8, and 2-13, and formulations 3-3, 3-8, and 3-13, the first emulsifier was sunflower seed phospholipid Lipoid HT00, the second emulsifier was polysorbate 80, the antioxidant was α-tocopherol, and the base oil was corn oil.

In formulations 1-4, 1-9, and 1-14, formulations 2-4, 2-9, and 2-14, and formulations 3-4, 3-9, and 3-14, the first emulsifier was polyene phosphatidylcholine, the second emulsifier was sorbitan oleate 80, the antioxidant was α-tocopherol, and the base oil was olive oil.

In formulations 1-5, 1-10, and 1-15, formulations 2-5, 2-10, and 2-15, and formulations 3-5, 3-10, and 3-15, the first emulsifier was soybean phospholipid Lipoid S75, the second emulsifier was sorbitan oleate 80, the antioxidant was α-tocopherol, and the base oil was olive oil.

In Table 2, “particle size” refers to the average particle size of the droplets in various batches of emulsions prepared with the same formulation and the same process.

### 1.1.2. Preparation

The oil-soluble components such as EPA-EE, the emulsifier, the antioxidant, etc. were introduced to a container according to the amounts in Table 2, preheated to about 50° C. to 70° C., and stirred vigorously until well dispersed, thereby forming an oil phase matrix. The water-soluble components such as a pH adjuster and a flavoring agent were introduced to another container according to the amounts in Table 2, preheated to about 50° C. to 70° C., and stirred vigorously until uniformly dispersed, thereby forming an aqueous phase matrix. Under stirring with a stator-rotor shearer at 7000 rpm to 10000 rpm, the oil phase matrix was added to the aqueous phase matrix and stirred for 3 min to 5 min until uniformly dispersed to form a milky-white primary emulsion, and water was added as a balance to obtain a total amount of 1000 mL. Then the primary emulsion was homogenized and emulsified at a pressure in the range from 200 bars to 800 bars for 6 to 10 times until the average particle size of the sample was below 300 nm. Then the emulsion was subjected to filtration with a 0.65 μm microporous filter membrane. The particle sizes of the obtained emulsions were tested and the results are shown in Table 2. The obtained emulsions were encapsulated with nitrogen filled, and sterilized at an elevated temperature of 115° C. for 30 min to obtain nano-lipid formulations, which were stored at room temperature.

### Example 1.2. Preparation of EPA-EE Nano-Lipid Formulations Containing Different Emulsifiers, Stabilizers, or Auxiliary Materials that Promotes EPA to Bind with Lipoproteins

The components were weighted according to the amounts in Table 3, and the nano-lipid formulations were prepared according to the method described in section 1.1.2 of Example 1.1. The prepared nano-lipid formulations were stored at room temperature.

TABLE 3
Compositions and particle sizes of different EPA-EE nano-lipid formulations

| | EPA- | First | | First | | Particle |
|---|---|---|---|---|---|---|
| Formulation | EE 60 | emulsifier | Stabilizer | auxiliary | | size |
| No. | (g) | (g) | (g) | material (g) | Water | (nm) |
| 4-1 to 4-5 | 100 | 10 | 10 | 0 | balance to 1 kg | 70~140 |
| 4-6 to 4-10 | 100 | 10 | 10 | 10 | balance to 1 kg | 170~290 |
| 4-11 to 4-15 | 200 | 100 | 20 | 20 | balance to 1 kg | 160~280 |
| | EPA- | First | | First | | Particle |
| Formulation | EE 80 | emulsifier | Stabilizer | auxiliary | | size |
| No. | (g) | (g) | (g) | material (g) | Water | (nm) |
| 5-1 to 5-5 | 100 | 10 | 10 | 0 | balance to 1 kg | 160~240 |
| 5-6 to 5-10 | 100 | 10 | 10 | 10 | balance to 1 kg | 160~260 |
| 5-11 to 5-15 | 200 | 100 | 20 | 20 | balance to 1 kg | 170~290 |
| | EPA- | First | | First | | Particle |
| Formulation | EE 97 | emulsifier | Stabilizer | auxiliary | | size |
| No. | (g) | (g) | (g) | material (g) | Water | (nm) |
| 6-1 to 6-5 | 100 | 10 | 10 | 0 | balance to 1 kg | 60~150 |
| 6-6 to 6-10 | 100 | 10 | 10 | 10 | balance to 1 kg | 120~180 |
| 6-11 to 6-15 | 200 | 100 | 20 | 20 | balance to 1 kg | 90~230 |

The formulations in Table 3 each further included 1 g of α-tocopherol as an antioxidant and 30 g of olive oil in addition to the above components.

In formulations 4-1, 4-6, and 4-11, formulations 5-1, 5-6, and 5-11, and formulations 6-1, 6-6, and 6-11, the first emulsifier was soybean phospholipid Lipoid S75, the stabilizer was TPGS1000, and the first auxiliary material was phosphatidylserine.

In formulations 4-2, 4-7, and 4-12, formulations 5-2, 5-7, and 5-12, and formulations 6-2, 6-7, and 6-12, the first emulsifier was soybean phospholipid Lipoid S75, the stabilizer was DSPE-PEG, and the first auxiliary material was sodium glutamate.

In formulations 4-3, 4-8, and 4-13, formulations 5-3, 5-8, and 5-13, and formulations 6-3, 6-8, and 6-13, the first emulsifier was soybean phospholipid Lipoid S75, the stabilizer was S40, and the first auxiliary material was phosphatidylserine.

In formulations 4-4, 4-9, and 4-14, formulations 5-4, 5-9, and 5-14, and formulations 6-4, 6-9, and 6-14, the first emulsifier was soybean phospholipid Lipoid S75, the stabilizer was S40, and the first auxiliary material was taurine.

In formulations 4-5, 4-10, and 4-15, formulations 5-5, 5-10, and 5-15, and formulations 6-5, 6-10, and 6-15, the first emulsifier was soybean phospholipid Lipoid S75, the stabilizer was a mixture of TPGS1000 and S40 in equal masses, and the first auxiliary material was a mixture of taurine and sodium glutamate in equal masses.

TABLE 4
Compositions and particle sizes of different EPA-EE nano-lipid formulations

| | EPA- | First | | First | | Particle |
|---|---|---|---|---|---|---|
| Formulation | EE 60 | emulsifier | Stabilizer | auxiliary | | size |
| No. | (g) | (g) | (g) | material (g) | Water | (nm) |
| 7-1 to 7-5 | 100 | 10 | 10 | 0 | balance to 1 kg | 70~150 |
| 7-6 to 7-10 | 100 | 10 | 10 | 10 | balance to 1 kg | 160~280 |
| 7-11 to 7-15 | 200 | 100 | 20 | 20 | balance to 1 kg | 170~270 |
| | EPA- | First | | First | | Particle |
| Formulation | EE 80 | emulsifier | Stabilizer | auxiliary | | size |
| No. | (g) | (g) | (g) | material (g) | Water | (nm) |
| 8-1 to 8-5 | 100 | 10 | 10 | 0 | balance to 1 kg | 150~220 |
| 8-6 to 8-10 | 100 | 10 | 10 | 10 | balance to 1 kg | 150~240 |
| 8-11 to 8-15 | 200 | 100 | 20 | 20 | balance to 1 kg | 160~270 |
| | EPA- | First | | First | | Particle |
| Formulation | EE 97 | emulsifier | Stabilizer | auxiliary | | size |
| No. | (g) | (g) | (g) | material (g) | Water | (nm) |
| 9-1 to 9-5 | 100 | 10 | 10 | 0 | balance to 1 kg | 60~140 |
| 9-6 to 9-10 | 100 | 10 | 10 | 10 | balance to 1 kg | 110~180 |
| 9-11 to 9-15 | 200 | 100 | 20 | 20 | balance to 1 kg | 100~220 |

The formulations in Table 4 each further included 2 g of α-tocopherol as an antioxidant and 30 g of a medium chain triglyceride as a base oil in addition to the above components.

In formulations 7-1, 7-6, and 7-11, formulations 8-1, 8-6, and 8-11, and formulations 9-1, 9-6, and 9-11, the first emulsifier was soybean phospholipid Lipoid S75, the stabilizer was TPGS1000, and the first auxiliary material was phosphatidylserine.

In formulations 7-2, 7-7, and 7-12, formulations 8-2, 8-7, and 8-12, and formulations 9-2, 9-7, and 9-12, the first emulsifier was soybean phospholipid Lipoid S75, the stabilizer was DSPE-PEG, and the first auxiliary material was sodium glutamate.

In formulations 7-3, 7-8, and 7-13, formulations 8-3, 8-8, and 8-13, and formulations 9-3, 9-8, and 9-13, the first emulsifier was soybean phospholipid Lipoid S75, the stabilizer was S40, and the first auxiliary material was phosphatidylserine.

In formulations 7-4, 7-9, and 7-14, formulations 8-4, 8-9, and 8-14, and formulations 9-4, 9-9, and 9-14, the first emulsifier was soybean phospholipid Lipoid S75, the stabilizer was S40, and the first auxiliary material was taurine.

In formulations 7-5, 7-10, and 7-15, formulations 8-5, 8-10, and 8-15, and formulations 9-5, 9-10, and 9-15, the first emulsifier was soybean phospholipid Lipoid S75, the stabilizer was a mixture of TPGS1000 and S40 in equal masses, and the first auxiliary material was a mixture of taurine and sodium glutamate in equal masses.

The “particle size” in tables 3 and 4 refers to the average particle size of the droplets in various batches of the emulsions prepared with the same formulation and process, and has the same meaning in the following examples.

### Example 1.3. Preparation of EPA-EE Nano-Lipid Formulations Using a Phospholipid with a Low Iodine Value

100 g of EPA-EE (97%) and 10 g of soybean powder phospholipid (with an iodine value of 64.5) were mixed, added with water to 1 kg, and prepared into a nano-lipid formulation according to the method of Example 1.1. The measured particle size of the prepared formulation was ranged from 750 nm to 900 nm, which is much higher than the particle size of other formulations in Examples 1 to 2, so the subsequent research work of Example 1.3 was terminated.

The TPGS used in Tables 2, 3, and 4, i.e., the TPGS used in the compositions for the formulations in individual tables were from the same manufacturer.

### Comparative Examples

Comparative Example 1 (referred to as D1): 100 g of ordinary fish oil (with an EPA content of 18.2%) and 10 g of soybean phospholipid were mixed, added with water to 1 kg, and prepared into a nano-lipid formulation according to the method of Example 1. The formulation was stored at room temperature until the evaluation was conducted.

Comparative Example 2 (referred to as D2): market product Vascepa®, EPA-EE capsules with a purity of above 97% developed by Amarin corporation (US).

Comparative Example 3 (referred to as D3): 2.12 g of water, 18 g of polyoxyethylene (20) sorbitan oleate ester, 18 g of polyethylene glycol 35 castor oil, 11 g of soybean lecithin, and 204.6 g of EPA-EE (a content of EPA in the raw material was 97%) were weighed, sealed, heated to about 70° C., and mixed to prepare a self-emulsifying composition (as Example 1 of patent document CN201680006802)

### 2. EFFECT EVALUATION

The “drug” involved herein refers to a component providing EPA.

### Example 2.1. In-Vitro Release of Lipid Formulations Rich in EPA-EE

Simulated fasting state intestinal liquid: 100 mM Tris, 300 mM NaCl, 10 mM CaCl₂), 10 mM sodium taurocholate, 2.5 mM phospholipid (soybean phospholipid S100 from Lipoid, Germany), and purified water in a corresponding proportion were stirred until dissolved and mixed uniformly. The pH of the resulting solution was adjusted to 7.5±0.05 using 0.5 g/mL maleic acid water solution. 100 mg of porcine pancreatic lipase was added to 1 mL of the above prepared solution to obtain an in-vitro simulated fasting state digestion buffer.

Equal amounts of experimental group formulations (formulation 4-1, formulation 1-6, formulation 1-11, formulation 2-6, formulation 3-6, and formulation 4-6) and control group formulations (formulation D1 in control example 1 and formulation D2 in control example 2) were respectively filled in dialysis bags. The bags were then sealed at both ends and placed in beakers containing 200 mL of the simulated intestinal liquid, and maintained at a temperature of 37° C. at a rotation speed of 100 rpm. 10 ml (V_{sampled}) of the intestinal liquid was sampled at intervals (0.25 h, 0.5 h, 1 h, 2 h, and 4 h), and replenished in time with the corresponding amount and temperature of dissolution medium. The in-vitro release was analyzed by gas chromatography after methyl esterification.

The experimental parameters for the methyl esterification were as follows.

Step 1: A clean glass test tube with a lid was filled with 100 μL of 300 μg/mL methyl heneicosanoate (an internal standard), dried with N2, then added with 100 μL of the liquid to be tested, 2 mL of 0.5 mol/L KOH-MeOH solution, and 0.5 mL of isooctane solution of BHT. The tube was sealed with the lid, vortexed for 60 sec to mix uniformly, and then left to stand for 10 to 15 min. The isooctane layer was collected and transferred to a clean injection vial containing a small amount of anhydrous sodium sulfate.

Step 2: 2 mL of 5% H₂SO₄-MeOH solution was added to the lower solution in step 1. After the surface of the solution was flushed with N2, the tube was sealed, vortexed to mix uniformly, reacted in water bath at 70° C. for 30 min, then taken out and cooled to about 40° C., added with 0.5 mL of isooctane, vortexed for 30 sec, then added with 0.5 mL of saturated sodium chloride solution, and vortexed for 15 sec. The isooctane layer was collected, combined with the organic layer from step 1, dried with anhydrous sodium sulfate, and transferred to an injection vial with an injection sleeve as a test solution.

The instrument for gas chromatography was an Agilent 7890A gas chromatograph instrument. The parameters of the gas chromatography test were as follows: (88%-cyanopropyl) aryl-polysiloxane capillary column (60 m×0.25 mm×0.2 μm); the temperature was increased by program from 170° C. at 0 min to 240° C. at the rate of 3.5° C./min; the temperature was kept at 240° C. for 10 min; the injector temperature was 250° C., and the detector temperature was 270° C.; the carrier gas was helium at a flow rate of 1.0 mL/min; the split ratio was 10:1; the injection volume was 1 μL.

The drug release percentage at a time point (t) was calculated by following formula:

$$Accumulated release ( \% ) = \frac{\begin{matrix}{V}_{total of system} \times {C}_{t test concentration} + \\ {\sum }_{0}^{t} ( {V}_{sampled} \times {C}_{t - 1 test concentration} )\end{matrix}}{{M}_{total}} \times 100 \%$$

Wherein V_{total of system} refers to 200 mL, V_{sampled} refers to 10 mL, C_{t test concentration} refers to the detected concentration of the drug at the time point t, Cₜ₋₁ test concentration refers to the detected concentration of the drug at the previous time point, and Mₜₒₜₐₗ refers to the total amount of EPA-EE contained in the formulation.

The drug release percentages at different time points are shown in Table 5. The simulated fasting state intestinal liquid contained fewer digestive enzymes. When EPA-EE was encapsulated in capsules (formulation D2), its release in the fasting state intestinal liquid was slow, only 49.5% after 2 h, and it cannot be completely released after 4 h. In contrast, when EPA-EE was administered in the emulsion form (all experimental groups), its release in the fasting state intestinal liquid was rapid, exceeding 80% after 1 h. This is correlated with the larger specific surface area provided by the nano-emulsion. The rapid release of EPA-EE in the intestinal environment facilitates quick absorption into blood after oral administration, allowing the compound to exert its effects more efficiently. In addition, the different emulsifiers and auxiliary materials in Table 3 all ensure good release of the nano-lipid formulation in the intestinal environment.

TABLE 5
In-vitro release of EPA in simulated intestinal digestive liquid (n = 3)

| Group | 0.25 h | 0.5 h | 1 h | 2 h | 4 h |
|---|---|---|---|---|---|
| Formulation 1-1 | 30.6 ± 3.3 | 69.5 ± 2.5 | 85.9 ± 3.8 | 88.7 ± 3.5 | 92.2 ± 3.3 |
| Formulation 1-6 | 36.6 ± 3.7 | 75.8 ± 3.1 | 89.1 ± 3.2 | 91.5 ± 3.6 | 92.6 ± 3.3 |
| Formulation 1-11 | 38.5 ± 2.7 | 77.4 ± 4.1 | 87.9 ± 3.5 | 92.5 ± 3.5 | 93.2 ± 3.3 |
| Formulation 2-6 | 35.5 ± 2.1 | 78.1 ± 3.1 | 86.9 ± 2.5 | 88.5 ± 2.5 | 89.2 ± 2.3 |
| Formulation 3-6 | 39.4 ± 3.9 | 75.5 ± 3.8 | 83.5 ± 3.2 | 85.5 ± 3.6 | 87.2 ± 3.9 |
| Formulation 4-6 | 31.0 ± 3.5 | 80.7 ± 3.1 | 85.7 ± 1.7 | 87 ± 3.65 | 90.2 ± 3.1 |
| Formulation D1 | 45.7 ± 3.1 | 77.5 ± 3.1 | 83.7 ± 5.1 | 84.6 ± 3.0 | 85.0 ± 1.8 |
| Formulation D2 | 24.5 ± 5.6 | 33.7 ± 2.5 | 40.5 ± 4.0 | 49.5 ± 4.5 | 62.2 ± 2.2 |

### Example 2.2. Pharmacokinetic Study on Different EPA-EE Nano-Lipid Formulations in Rats

Fifty-four Sprague-Dawley (SD) male rats, each weighing 200 g±20 g, six in each group, were respectively intragastrically administered with the experimental group formulations (formulation 4-1, formulation 1-6, formulation 1-11, formulation 2-6, formulation 3-6, and formulation 4-6) and the control group formulations (formulation D1 in control example 1 and formulation D2 in control example 2). The converted dosage of EPA-EE was 400 mg/kg. Blood samples of 0.5 mL were collected at the following time points after the oral administration: 0.5 h, 1 h, 2 h, 3 h, 4 h, 6 h, 8 h, 10 h, 12 h, and 24 h. The blood samples were placed in centrifuge tubes containing 1% (w/v) sodium heparin. The plasma was separated by centrifugation at 3000 rpm for 10 min at a temperature below 4° C., and the supernatant was taken and stored at −20° C. until the drug concentration was measured.

The content of EPA in plasma was detected by gas chromatography, and the results were analyzed statistically using Graphpad Prism software. A T-test was used to compare the results between two groups, while analysis of variance and multiple comparisons were performed for multiple groups. The experimental data are expressed as mean±SD, where SD stands for standard deviation.

The instrument and parameters for gas chromatography were the same as in Example 2.1.

TABLE 6
Pharmacokinetic evaluation of formulations in various groups

| | | Cₘₐₓ | AUC_{0~24 h} | Maintenance time | Maintenance time |
|---|---|---|---|---|---|
| Group | Tₘₐₓ (h) | (μg/mL) | (μg/mL · h) | for 200 μg/mL (h) | for 100 μg/mL (h) |
| Formulation 4-1 | 2 | 570.9 ± 34.8 | 2576.3 | 2.13 | 9.04 |
| Formulation 5-1 | 2 | 762.2 ± 34.8 | 3092.5 | 2.52 | 9.38 |
| Formulation 6-1 | 2 | 1429.5 ± 48.7 | 4209.5 | 3.45 | 13.01 |
| Formulation 6-2 | 2 | 1223.2 ± 31.6 | 3892.5 | 3.29 | 12.23 |
| Formulation 6-3 | 2 | 812.1 ± 24.4 | 3039.4 | 2.85 | 9.54 |
| Formulation 6-4 | 2 | 1223.2 ± 31.6 | 3892.5 | 3.29 | 12.23 |
| Formulation 6-6 | 2 | 1485.5 ± 48.7 | 4809.5 | 4.05 | 14.21 |
| Formulation D1 | 2 | 188.6 ± 21.2 | 1205.5 | 0.00 | 6.98 |
| Formulation D2 | 4 | 206.2 ± 75.2 | 1056.2 | 0.51 | 4.12 |

After administering nine formulations via oral gavage to SD rats, the contents of EPA in the blood were detected at the sampling time points and the results are shown in Table 6. According to the results, the time to reach peak plasma drug concentration of the experimental groups and control group 1 (formulation D1) was 2 hours earlier than that of control group 2 (formulation D2, the capsule group), which indicates that the nano-lipid formulation provided in the present application has a significant advantage in promoting absorption. Moreover, in formulations D1, 4-1, 5-1, and 6-1, the peak drug concentration and bioavailability were increased with the EPA-EE content, indicating that the EPA-EE nano-lipid formulation with a high purity of EPA (experimental groups) is more superior. In all experimental groups, the EPA concentration was maintained above 200 μg/mL for over 2.0 h and maintained above 100 μg/mL for over 9 h.

Different types of stabilizers were adopted in formulations 6-1, 6-2, 6-3, and 6-4. For the emulsions with the stabilizers TPGS, DSPE-PEG, and S40, the EPA concentration was maintained above 200 g/mL for over 3 h, and was maintained above 100 μg/mL for over 10 h, However, formulation 6-4 using F68 as the stabilizer did not show significant stabilizing effects. This indicates that the stabilizers having a terminal group provided by a lipophilic PEG chain are more effective in maintaining the blood drug concentration, leading to a higher bioavailability (see data of AUC0-24 h). In addition, formulation 6-6, which further included taurine on basis of formulation 6-1, can promote the lipoprotein binding and facilitate insertion of EPA into lipoproteins to maintain the concentration, and thus demonstrated the best result.

### Example 2.3. Investigation on EPA Content in Lipoproteins in Rats with Different EPA-EE Nano-Lipid Formulations

Fifty-four Sprague-Dawley (SD) male rats, each weighing 200±20 g, six in each group, were fed freely with a basal diet for one week to adapt to the environment. After that, the rats were randomly divided into a blank control group and a model group. The blank group was fed a basal diet while the model group was fed a high-fat diet for two weeks to complete the modeling. Then the rats were respectively intragastrically administered with the experimental group formulations (formulation 4-1, formulation 5-1, formulation 6-1, formulation 6-2, formulation 6-3, formulation 6-4, formulation 6-5, and formulation 6-6) and the control group formulations (formulation D1 in control example 1 and formulation D2 in control example 2). The converted dosage of EPA oil phase was 400 mg/kg. The rats were intragastrically administered once a day, with free access to food and water, until the end of the 11th week (8 weeks of drug administration).

After the drug administration, blood was collected from the heart of each rat in each group under anesthesia. The blood was placed into centrifuge tubes containing 1% (w/v) sodium heparin, and centrifuged at 3000 rpm for 10 min at a low temperature to separate the plasma. Apolipoproteins were separated from the plasma by iodixanol density gradient centrifugation. The lipids were extracted by acid/methanol/chloroform extraction, centrifuged, purified by isohexane and solid-phase extraction, and confirmed to be fully hydrolyzed and transmethylated of lipids (acid/methanol, 50° C. overnight). EPA was fully titrated, and the EPA concentration was measured using a validated liquid chromatography/tandem mass spectrometry method. The test results were analyzed statistically using Graphpad Prism software. A T-test was used to compare the results between two groups, while analysis of variance and multiple comparisons were performed for multiple groups. The experimental data are expressed as mean±SD, where SD stands for standard deviation, and P<0.05 was considered statistically significant.

Method for testing EPA content: The full titration of EPA was based on the formation of methyl ester of EPA during transmethylation. For unesterified EPA, a solution of Lovan inhibitor (containing 0.5 g sodium fluoride, 1.0 g L-ascorbic acid, and 0.25 g 5-methylisoxazole-3-carboxylic acid per 10 mL water) was added to each 1 mL of the plasma sample to prevent degradation. The lipids were extracted with methanol/chloroform (without hydrolysis or methylation), centrifuged to separate, and then purified by protein precipitation and solid-phase extraction. The EPA concentration was measured using a validated liquid chromatography/tandem mass spectrometry method (Charles River Laboratories Ltd, Elphinstone Research Center, Tranent, Scotland, UK). The analyte was separated by a Perkin Elmer liquid chromatography system (Perkin Elmer, Beaconsfield, Cheshire, UK) utilizing an ascesis R Express C18 column: 2.7 mm (Sigma-Aldrich Co. Ltd, Poole, UK) at a flow rate of 1 mL/min, a column temperature of 60° C., and mobile phases of 60%/40% (A/B) to 100% A. The mobile phase A was acetonitrile/acetic acid (100/0.5, v/v) and the mobile phase B was water/acetic acid (100/0.5, v/v).

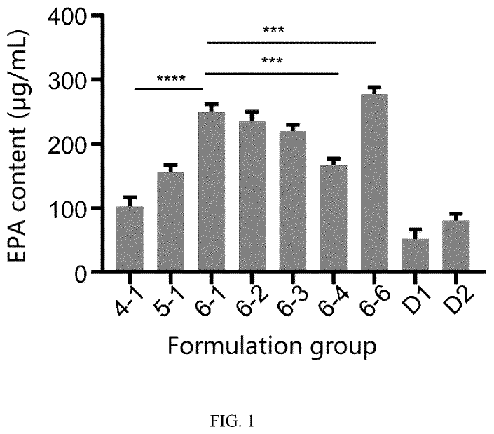

The experimental results are shown in FIG. 1. After oral gavage of the seven formulations in SD rats, significant differences can be observed in the EPA content in low-density lipoprotein. The purity of EPA in control group 1 (formulation D1) was low (about 20%), and thus the content of EPA in low-density lipoprotein was low, despite the lipid formulation promoting absorption. In control group 2 (formulation D2), the capsule formulation exhibited slow absorption and fast metabolism, resulting in low EPA content in low-density lipoprotein. In formulation groups 4-1, 5-1, and 6-1, which were nano-emulsions containing more than 60% EPA-EE, the use of different emulsifiers showed no significant effect on the binding of EPA to low-density lipoprotein. With the effective stabilizers, such as in formulation groups 6-1, 6-2, and 6-3, the high EPA concentrations in blood were maintained, prolonging the residence time of EPA in blood and making it not easily to be cleared, creating conditions for the accumulation of EPA in low-density lipoprotein. In addition, formulation group 6-6 contains the auxiliary material that competitively binds with the lipoprotein, promoting the active enrichment of EPA in the lipoprotein.

### Example 2.4. Investigation on Blood Lipid Lowering Effect of Different EPA-EE Nano-Lipid Formulations

SD male rats (Shanghai Laboratory Animal Research Center), each weighing 200±20 g, were fed freely with a basal diet for one week to adapt to the environment. After that, the rats were randomly divided into a blank control group and a model group. The blank control group was fed a basal diet while the model group was fed a high-fat diet for two weeks to complete the modeling.

After the modeling was completed, the rats in the blank control group and the model group were intragastrically administered with deionized water every day, and in the other groups were respectively administered with the experimental group formulations (formulation 3-1, formulation 3-2, formulation 3-6, formulation 6-1, formulation 6-2, formulation 6-6, and formulation 6-7) and the control group formulations (formulation D1 in control example 1 and formulation D2 in control example 2) for 28 days, with converted dosage of EPA-EE of 400 mg/kg. On the 14^{th} day and the 28^{th} day of the drug administration, the rats were fasted for 4 h, and whole blood samples (not less than 0.5 mL) were collected from the rats under anesthesia. The whole blood samples were centrifuged at 4000 rpm for 15 min at 4° C., and the supernatant serum was taken. The absorbance at the wavelength of 510 nm was measured using an ultraviolet-visible spectrophotometer. The total cholesterol (TC) in the serum was determined by using the CHOD-PAP method, and the triglyceride (TG) in the serum was determined by using the GPO-PAP method.

CHOD-PAP method: A total cholesterol assay kit (Jiangsu Innova Medical Technology Co., Ltd.) was used. The working solution was prepared by mixing an enzyme reagent with a dilution solution at a ratio of 1:4. The centrifuge tubes for the blank group, the standard group, and the test group were filled with the following reagents in sequence: 10 μL of distilled water, 10 μL of a standard solution (cholesterol solution at different concentrations), 10 μL of a serum standard solution, and 10 μL of the enzyme working solution. After mixing uniformly, the centrifuge tubes were incubated in a water bath at 37° C. for 15 min. After calibration with the blank, the absorbance of each tube at the wavelength of 510 nm was read, and the concentration of total cholesterol in the serum was calculated using the standard curve.

GPO-PAP method: A triglyceride assay kit (Beijing Regen Biotechnology Co., Ltd.) was used. The centrifuge tubes for the blank group, the standard group, and the test group were filled with the following reagents in sequence: 10 μL of distilled water, 10 μL of a standard solution (triglyceride solution of different concentrations), 10 μL of a serum standard solution, and 10 μL of the enzyme working solution standard solution. After mixing uniformly, the centrifuge tubes were incubated in a water bath at 37° C. for 15 min. After calibration with the blank, the absorbance of each tube at the wavelength of 510 nm was read, and the concentration of triglyceride in the serum was calculated according to the following formula: TG(mmol/L)={(absorbance of test tube−absorbance of blank)/(absorbance of standard tube−absorbance of blank)}×1.7 mmol/L.

TABLE 7
Lipid-lowering effects of different groups (TC and TG levels)

| | 14th day | | 28th day | |
|---|---|---|---|---|
| Formulation | TC (mmol/L) | TG (mmol/L) | TC (mmol/L) | TG (mmol/L) |
| Blank group | 1.33 ± 0.11 | 0.93 ± 0.23 | 1.35 ± 0.14 | 0.93 ± 0.12 |
| Model group | 2.89 ± 0.15 | 4.51 ± 0.26 | 2.94 ± 0.09 | 4.98 ± 0.73 |
| Formulation 3-1 | 2.69 ± 0.31 | 3.23 ± 0.20 | 1.89 ± 0.08 | 2.86 ± 1.16 |
| Formulation 3-2 | 2.61 ± 0.27 | 3.07 ± 0.32 | 1.73 ± 0.22 | 2.66 ± 0.29 |
| Formulation 3-6 | 2.76 ± 0.17 | 3.32 ± 0.26 | 1.93 ± 0.17 | 2.81 ± 0.31 |
| Formulation 6-1 | 2.25 ± 0.28 | 2.78 ± 0.40 | 1.62 ± 0.14 | 1.78 ± 0.86 |
| Formulation 6-2 | 2.36 ± 0.21 | 2.99 ± 0.41 | 1.79 ± 0.40 | 1.90 ± 0.64 |
| Formulation 6-6 | 2.02 ± 0.38 | 3.43 ± 0.34 | 1.51 ± 0.14 | 1.34 ± 0.86 |
| Formulation 6-7 | 2.06 ± 0.28 | 2.40 ± 0.34 | 1.44 ± 0.14 | 1.56 ± 0.86 |
| Formulation D1 | 2.76 ± 0.31 | 3.43 ± 0.20 | 1.91 ± 0.08 | 3.05 ± 1.16 |
| Formulation D2 | 2.63 ± 0.18 | 4.45 ± 0.28 | 2.19 ± 0.08 | 3.89 ± 0.46 |

The experimental results are shown in Table 7. The EPA-EE nano-lipid compositions demonstrate good blood lipid-lowering effects. The phospholipid emulsifiers used in formulations 3-1, 3-2, and 3-6 have different iodine values, and formulation 3-2 with the higher iodine value can exert the best blood lipid-regulating effect, resulting in lower levels of TC and TG in the rat blood. In addition, the formulations 6-1 and 6-2 containing more effective stabilizers exhibit more significant blood lipid-regulating effects compared to formulation 3-2. Furthermore, the formulations 6-6 and 6-7 containing the auxiliary material promoting the lipoprotein binding further amplified the blood lipid-lowering effects of the nano-lipid composition.

By observing the experimental animals during the experiment, except for the model group, all rats exhibited normal activities, with no abnormality in their physical appearances or feces. After four weeks of administration, there was no statistically significant difference in the body weight among the groups. These experimental results confirmed the safety of the nano-lipid formulations.

### Example 2.5. Pharmacodynamic Study on Plaque Reduction with Different EPA-EE Nano-Lipid Formulations

120 SPF-grade male ApoE^{-/-} mice (each weighing 18 g to 22 g), 6-8 weeks of age, were randomly housed in mouse cages and fed with a basal diet for one week to adapt to the environment. After one week, if no abnormality was observed, the rats were fed with a high-fat diet for 12 weeks, with free access to food and water, to develop an arterial plaque model. At the end of week 12, mice were randomly selected and processed for pathology analysis, and then H&E staining was performed to observe the presence or absence of plaques and the morphology of the plaques to confirm the successful modeling. Upon confirming the successful modeling, the remaining modeling mice were further sorted by weight and randomly assigned to 11 groups at the end of week 13, including: an atorvastatin group (PD, positive control group), experimental groups (formulation 2-1, formulation 3-1, formulation 3-6, formulation 6-1, formulation 6-2, formulation 6-6), formulation control groups (formulation D1, formulation D2), and a model group (M, given equal amounts of water). Additionally, 10 healthy mice, without modeling, were assigned to a control group (N).

After weighing the mice in each group, the drug intervention was started at week 13 by intragastric administration. The dosing regimen for each group of mice was as follows. The PD group was administered with 5 mg/kg/dose of atorvastatin; the experimental group and the formulation control group were administered with the formulations equivalent to 100 mg/kg/dose of EPA-EE; the model group (M) and the model control group (N) were administered with equal volumes of distilled water, which was the same as used in the preparation of the drug. The rats were intragastrically administered twice daily with free access to food and water, until the end of the 12th week (8 weeks of drug administration). Body weight was recorded once a week after the start of the drug intervention. Twenty-four hours after the final intervention, the mice in each group were weighed again, and then pathologically sampled. Subsequently, the mice were anesthetized with ether, and the mice's eyes were removed for blood collection. Following the blood collection, the mice were euthanized by cervical dislocation, the thoracic cavity was quickly opened, and the aorta was subjected to blunt dissection for visual inspection and photography. The aorta was fixed in 4% formaldehyde solution, sectioned, stained with H&E, observed and photographed using an optical microscope. Image ProPlus 6.0 computer image analysis system was used to analyze and calculate the vessel area and the plaque area, as well as the percentage of the plaque area relative to the vessel area.

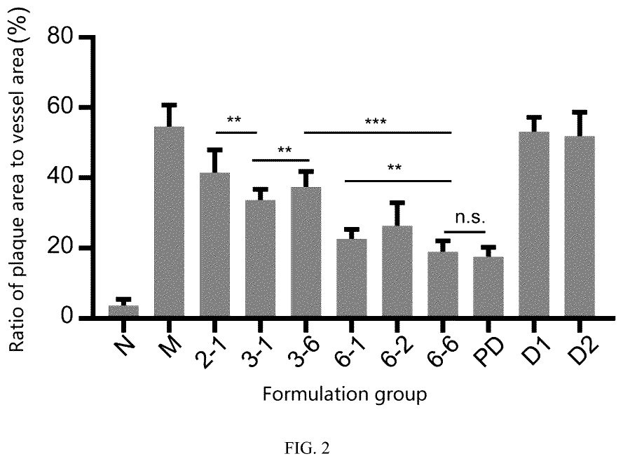

The results are shown in FIG. 2. Compared with the model group (M), EPA-EE encapsulated by the lipid formulations slowed the progression of arterial plaques, and the higher the purity of the raw material, the smaller the plaque area. Consistent with the blood lipid regulation results, better treatment effects can be achieved by the groups containing the emulsifier with the higher iodine value (formulation 3-1) and containing the derivative with the non-ionic polymer segment (formulations 6-1, 6-2, and 6-6). In particular, as containing the auxiliary material which promotes the binding of EPA-EE to low-density lipoprotein, formulation 6-6 demonstrated efficacy approaching that of the positive control group PD using the statin. The above experimental groups significantly slowed the development of arterial plaques in comparison with the control groups (D1 and D2).

### Example 2.6. Pharmacokinetic Study on Different EPA-EE Nano-Lipid Formulations in Beagles

Twenty-one male beagles, each weighing 8 kg to 12.5 kg, three in each group, were respectively intragastrically administered with the experimental group formulations (formulation 6-1, formulation 7-1, formulation 8-1, formulation 9-1, and formulation 9-6) and the control group formulations (formulation D2 in control example 1 and formulation D3 in control example 2). The converted dosage of EPA-EE was 120 mg/kg. Blood samples of 2 mL were collected at 0.5 h, 1 h, 2 h, 3 h, 4 h, 6 h, 8 h, 10 h, 12 h, 24 h, and 48 h after oral administration and placed in centrifuge tubes containing 1% (w/v) sodium heparin. The plasma was separated by centrifugation at 3000 rpm for 10 min at a temperature below 4° C., and the supernatant was taken and stored at −20° C. until the drug concentration was measured.

The total content of EPA in plasma was detected by gas chromatography, and the results were analyzed statistically using Graphpad Prism software. A T-test was used to compare the results between two groups, and analysis of variance and multiple comparisons were performed for multiple groups. The experimental data are expressed as mean±SD, where SD stands for the standard deviation.

TABLE 8
Pharmacokinetic evaluation of formulations in various groups

| | | Cₘₐₓ | AUC_{0~48 h} | Maintenance time | Maintenance time |
|---|---|---|---|---|---|
| Group | Tₘₐₓ (h) | (μg/mL) | (μg/mL · h) | for 200 μg/mL (h) | for 60 μg/mL (h) |
| Formulation 6-1 | 3 | 212.6 ± 49.6 | 1984.9 | 0.5 | 5.0 |
| Formulation 7-1 | 2 | 198.3 ± 58.2 | 1668.4 | 0 | 4.7 |
| Formulation 8-1 | 2 | 179.2 ± 55.8 | 1519.5 | 0 | 4.5 |
| Formulation 9-1 | 2 | 251.5 ± 61.6 | 2012.7 | 1.2 | 6.8 |
| Formulation 9-6 | 2 | 246.8 ± 80.6 | 1992.6 | 0.9 | 6.9 |
| Formulation D2 | 4 | 29.8 ± 7.6 | 357.6 | 0 | 0 |
| Formulation D3 | 2 | 171.5 ± 54.6 | 1475.5 | 0 | 4.6 |

After administering seven formulations via oral gavage to beagles, the contents of EPA in the blood were detected at the sampling time points and the results are shown in Table 8. According to the results, the time to reach peak plasma drug concentration of the experimental groups and control group 2 (formulation D3) was 2 hours earlier than that of control group 1 (formulation D2, the capsule group), which indicates that the nano-lipid formulation provided in the present application has a significant advantage in promoting absorption. In the experimental groups, the EPA concentration was maintained above 60 μg/mL for over 4 h (≥4 h). At the same dosage, the Cₘₐₓ and AUC of the EPA-EE nano-lipid formulation in the present application were higher than those of control group 2 (formulation D3), suggesting that the formulation in the present application has a higher oral absorption and bioavailability.

### Example 2.7. Study on Viscosity and Lipid Hydrolysis Stability of Different EPA-EE Nano-Lipid Formulations

2.7.1 Viscosity comparison of formulations: The viscosity formulation 7-1, formulation 8-1, formulation 9-1, formulation 9-6, formulation D2, and formulation D3 were measured by using an NDJ-1 rotational viscometer, and the results are shown in Table 9.

TABLE 9
Results of viscosity of different formulations

| Group | Viscosity (mPa · s) |
|---|---|
| Formulation 7-1 | 2.5 |
| Formulation 8-1 | 2.6 |
| Formulation 9-1 | 2.4 |
| Formulation 9-6 | 2.5 |
| Formulation D2 | 63.2 |
| Formulation D3 | 58.9 |

The measurement results show that the formulations with EPA-EE prepared into an emulsion have a significantly decreased viscosity as compared to formulations D2 and D3.

2.7.2 Hydrolysis stability test: The capsule content of formulation D2 and formulation D3 were diluted with water to form water solutions of 100 mg/ml, and then placed at 60° C. for 30 days together with formulations 7-1, 8-1, and 9-1. After the 30 days, the EPA-EE contents in the water solutions of the formulations were measured by using gas chromatography. The results are shown in Table 10.

TABLE 10
Stability of water solutions of different formulations

| | Change of the EPA-EE content |
|---|---|
| Group | in 30 days |
| Formulation 7-1 | −1.1% |
| Formulation 8-1 | −0.5% |
| Formulation 9-1 | −0.6% |
| Formulation D2 | −31.2% |
| Formulation D3 | −12.5% |

The experimental results show that the EPA-EE content in formulation D2 decreased significantly, suggesting that EPA-EE is easy to hydrolyze in water. The self-emulsifying formulation of formulation D2 also exhibited significant hydrolysis in water. However, the EPA-EE content in formulation 7-1, formulation 8-1, and formulation 9-1 were substantially unchanged, suggesting that the formulation in the present application can effectively improve the chemical stability of EPA-EE and reduce the hydrolysis of EPA-EE.

The inventors of the present application have discovered through experiments that, even on the condition that the first auxiliary material was omitted, any one of the pharmaceutical compositions and the EPA-EE nano-lipid formulations containing the first auxiliary material in Tables 3 and 4 can still achieve a average droplet size less than or equal to 500 nm (further 100 nm to 300 nm) and can still significantly improve oral bioavailability (compared to the above comparative examples).

The technical features of the above-mentioned embodiments and examples can be combined arbitrarily. In order to make the description concise, not all possible combinations of the technical features of the above-mentioned embodiments and examples are described. However, as long as there is no contradiction in the combination of these technical features, the combinations should be considered as in the scope of the present application.

The above-described embodiments are only several implementations of the present application to facilitate a detailed understanding of the technical solutions of the present application, but they are not to be construed as limitations on the scope of protection of the invention patent. It should be noted that, for a person of ordinary skill in the art, several variations and improvements can be made without departing from the conception of the present application, all of which fall within the scope of protection of the present application. It should also be understood that after reading the above teachings of the present application, a person skilled in the art may make various changes or modifications to the present application, and the equivalent forms obtained will also fall within the scope of protection of the present application. It should also be understood that the technical solutions obtained by the person skilled in the art through logical analysis, reasoning, or limited experimentation on the basis of the technical solutions provided in this application are within the scope of protection of the claims appended to this application. Therefore, the scope of protection of the patent application shall be subject to the contents of the appended claims, and the specification and the accompanying drawings may be used to explain the contents of the claims.

1. An eicosapentaenoic acid ethyl ester (EPA-EE) nano-lipid composition comprising following components in parts by mass: 1 to 30 parts of an EPA-EE raw material, 0.1 to 10 parts of a first emulsifier, 0 to 10 parts of a second emulsifier, 0 to 5 parts of a stabilizer, 0 to 5 parts of a first auxiliary material, and 0 to 15 parts of a second auxiliary material,
wherein:
a mass content of EPA-EE in the EPA-EE raw material is greater than or equal to 60%;
the first emulsifier is a highly unsaturated phospholipid with an iodine value greater than or equal to 70;
a mass proportion of phosphatidylcholine in the highly unsaturated phospholipid is greater than or equal to 50%;
a composition of the second emulsifier is different from that of the first emulsifier, and the second emulsifier is selected from the group consisting of a food-acceptable raw material, a food-acceptable auxiliary material, a pharmaceutically acceptable raw material, a pharmaceutically acceptable auxiliary material, and any combination thereof;
the stabilizer is a non-ionic polymer;
the first auxiliary material is an auxiliary material promoting EPA to bind with a lipoprotein;
the second auxiliary material is selected from the group consisting of a food-acceptable raw material, a food-acceptable auxiliary material, a pharmaceutically acceptable raw material, a pharmaceutically acceptable auxiliary material, and any combination thereof, and the second auxiliary material is different from any one of the first emulsifier, the second emulsifier, the stabilizer, and the first auxiliary material.

2. The EPA-EE nano-lipid composition of claim 1, further comprising water, wherein:
an amount of water in the EPA-EE nano-lipid composition is greater than or equal to 65 parts by mass;
a total amount of the EPA-EE nano-lipid composition is 100 parts by mass.

3. The EPA-EE nano-lipid composition of claim 2, comprising following components in weight percentages based on a total weight of the EPA-EE nano-lipid composition:
the EPA-EE raw material 1% to 30% (w/w);
the first emulsifier 0.1% to 10% (w/w);
the second emulsifier 0% to 10% (w/w);
the stabilizer 0 to 5% (w/w);
the first auxiliary material 0 to 5% (w/w);
the second auxiliary material 0 to 15% (w/w); and
water;
wherein:
the weight percentage of water in the EPA-EE nano-lipid composition is at least 65% (w/w).

4. The EPA-EE nano-lipid composition of claim 3, satisfying any one or a combination of following features:
based on the total weight of the EPA-EE nano-lipid composition, a content of the stabilizer is in a range from 0.01% to 5%;
based on the total weight of the EPA-EE nano-lipid composition, a content of the first auxiliary material is in a range from 0.01% to 5% (w/w).

5. The EPA-EE nano-lipid composition of claim 3, satisfying any one or any combination of following features:
based on the total weight of the EPA-EE nano-lipid composition, a weight proportion of the second emulsifier is in a range from 0.1% to 10%;
based on the total weight of the EPA-EE nano-lipid composition, a weight proportion of the stabilizer is in a range from 0.1% to 5%;
based on the total weight of the EPA-EE nano-lipid composition, a weight proportion of the first auxiliary material is in a range from 0.1% to 5%;
based on the total weight of the EPA-EE nano-lipid composition, a weight proportion of the second auxiliary material is in a range from 0.01% to 15%.

6. The EPA-EE nano-lipid composition of claim 3, comprising following components in weight percentages based on the total weight of the EPA-EE nano-lipid composition:
the EPA-EE raw material 40 h to 200 (w/w);
the first emulsifier 0.5% to 5% (w/w);
the second emulsifier 0.5% to 5% (w/w);
the stabilizer 0.1% to 3% (w/w);
the first auxiliary material 0.1% to 3% (w/w);
the second auxiliary material 0.01% to 10% (w/w); and
water.

7. The EPA-EE nano-lipid composition of claim 3, satisfying any one or any combination of following features:
the EPA-EE raw material is an ethyl esterification product of one or more oils selected from the group consisting of a deep-sea fish oil, a seaweed oil, a krill oil, and any combination thereof,
the mass content of EPA-EE in the EPA-EE raw material is greater than or equal to 70%;
the iodine value of the highly unsaturated phospholipid is greater than or equal to 80;
the mass proportion of phosphatidylcholine in the highly unsaturated phospholipid is greater than or equal to 60%;
the first emulsifier is selected from the group consisting of soybean phospholipid, sunflower seed phospholipid, polyene phosphatidylcholine, and any combination thereof;
the stabilizer is an amphiphilic non-ionic polymer, and the stabilizer is selected from the group consisting of a vitamin lipid polymer derivative, a phospholipid polymer derivative, a fatty acid ester polymer derivative, and a polyoxyethylene-polyoxypropylene ether block copolymer, and any combination thereof;
a molecular weight of a PEG unit in the phospholipid polymer derivative is in a range from 400 Da to 6000 Da the molecular weight refers to a number-average molecular weight or a weight-average molecular weight;
a molecular weight of an PEG unit in the fatty acid ester polymer derivative is in a range from 200 Da to 4000 Da; the molecular weight refers to a number-average molecular weight or a weight-average molecular weight;
the first auxiliary material is selected from the group consisting of an amino acid with a side chain having a negatively charged group, an amino acid derivative with a negatively charged group, and a small peptide with a side chain having a negatively charged group, and any combination thereof,
the second auxiliary material is selected from the group consisting of an antioxidant, a base oil, a co-emulsifier, a flavoring agent, an interfacial membrane stabilizer, a thickener, a pH adjuster, and any combination thereof,
the EPA-EE nano-lipid composition is in form of a submicron emulsion containing droplets with an average particle size less than or equal to 500 nm.

8. The EPA-EE nano-lipid composition of claim 7, satisfying any one of or any combination of following features:
the iodine value of the first emulsifier is greater than 90, and the first emulsifier is selected from the group consisting of soybean phospholipid, sunflower seed phospholipid, polyene phosphatidylcholine, and any combination thereof,
the second emulsifier is different from the first emulsifier and is selected from the group consisting of phospholipid, sucrose ester, citric and fatty acid esters of glycerol, fatty acid glyceride, glyceryl monolinoleate, glycerol monostearate, polysorbate, sorbitan fatty acid ester, polyoxyethylene fatty acid ester, Span, alginate, sodium oleate, caseinate, and any combination thereof;
the stabilizer comprises a terminal group provided by the PEG unit, and the terminal group is OH or methoxy;
the vitamin lipid polymer derivative is selected from the group consisting of d-α-tocopheryl polyethylene glycol 200 succinate, d-α-tocopheryl polyethylene glycol 400 succinate, d-α-tocopheryl polyethylene glycol 1000 succinate, d-α-tocopheryl polyethylene glycol 1500 succinate, d-α-tocopheryl polyethylene glycol 2000 succinate, d-α-tocopheryl polyethylene glycol 4000 succinate, and any combination thereof;
the phospholipid polymer derivative is selected from the group consisting of distearoylphosphatidylethanolamine-polyethylene glycol 2000, distearoylphosphatidylethanolamine-polyethylene glycol 5000, dipalmitoylphosphatidylethanolamine-methoxypolyethylene glycol 2000, dipalmitoylphosphatidylethanolamine-methoxypolyethylene glycol 5000, soybean phosphatidylethanolamine-polyethylene glycol monomethyl ether 2000, 1,2-di-myristoyl-rac-glycero-3-methoxypolyethylene glycol 2000, dilauroylphosphatidylethanolamine-polyethylene glycol 2000, dioleoylphosphatidylethanolamine-polyethylene glycol, and any combination thereof;
the fatty acid ester polymer derivative is selected from the group consisting of polyethylene glycol 400 oleate, polyethylene glycol 600 oleate, polyethylene glycol 4000 oleate, polyethylene glycol 6000 oleate, polyethylene glycol 400 dioleate, polyethylene glycol 600 dioleate, polyethylene glycol 200 laurate, polyethylene glycol 200 dilaurate, polyethylene glycol 400 laurate, polyethylene glycol 400 dilaurate, polyethylene glycol 400 stearate, polyethylene glycol 400 distearate, and any combination thereof;
the polyoxyethylene-polyoxypropylene ether block copolymer is selected the group consisting of Pluronic L65, Pluronic F68, and a combination thereof,
in the first auxiliary material, the amino acid with the side chain having the negatively charged group is selected from the group consisting of aspartic acid, glutamic acid, taurine, and any combination thereof,
in the first auxiliary material, the amino acid derivative with the side chain having the negatively charged group is selected from the group consisting of phosphatidylserine, dihexadecyl-glutamate-glutamine, dihexadecyl-glutamate-glutamic acid, dihexadecyl-glutamate-asparagine, and any combination thereof,
in the first auxiliary material, the small peptide with the side chain having the negatively charged group is glutathione;
in the second auxiliary material, the antioxidant is selected from the group consisting of vitamin E, α-tocopherol, β-tocopherol, γ-tocopherol, mixed tocopherols, α-tocopheryl acetate, β-tocopheryl acetate, γ-tocopheryl acetate, mixed tocopheryl acetates, ascorbic acid, ascorbyl palmitate, ascorbyl stearate, ascorbyl myristate, sodium ascorbate, butylated hydroxyanisole, dibutylated hydroxytoluene, propylgallate, tert-butylhydroquinone, and any combination thereof,
in the second auxiliary material, the base oil is selected from the group consisting of soybean oil, olive oil, jojoba oil, sweet almond oil, grapeseed oil, corn oil, walnut oil, sea buckthorn oil, olive oil, coix seed oil, grapeseed oil, ginger oil, coconut oil, camellia oil, rose oil, peppermint oil, lemon oil, medium-chain triglycerides, and any combination thereof;
the EPA-EE nano-lipid composition is in form of a submicron emulsion containing droplets with an average particle size in a range from 100 nm to 300 nm.

9. The EPA-EE nano-lipid composition of claim 1, satisfying any one or any combination of following features:
the first emulsifier is selected from the group consisting of soybean phospholipid S75, soybean phospholipid S100, sunflower seed phospholipid H100, polyene phosphatidylcholine, and any combination thereof;
the second emulsifier is selected from the group consisting of egg yolk lecithin E80, polysorbate 80, sorbitan oleate 80, and any combination thereof;
the stabilizer is selected from the group consisting of TPGS, DSPE-PEG, S40, and any combination thereof;
the first auxiliary material is selected from the group consisting of phosphatidylserine, sodium glutamate, taurine, and any combination thereof;
the second auxiliary material comprises an antioxidant and a base oil.

10. The EPA-EE nano-lipid composition of claim 1, comprising following components in parts by mass: 50 to 500 parts by mass of the EPA-EE raw material, 10 to 100 parts by mass of the first emulsifier, 0 to 100 parts by mass of the second emulsifier, 0 to 1.2 parts by mass of α-tocopherol, 0 to 60 parts by mass of a base oil, and water; and
further satisfying one or more of following features:
a total amount of the EPA-EE nano-lipid composition is 900 to 1100 parts by mass;
the EPA-EE raw material takes 100 to 400;
the first emulsifier takes 10 to 100 parts by mass;
the first emulsifier comprises soybean phospholipid, sunflower seed phospholipid, polyene phosphatidylcholine, or a combination thereof
the second emulsifier takes 10 to 100 parts by mass,
the second emulsifier comprises egg yolk lecithin, polysorbate, sorbitan oleate 80, or a combination thereof;
the α-tocopherol takes 0.1 to 1 part by mass;
the base oil takes 30 to 50 parts by mass; and
the base oil comprises corn oil, olive oil, or a combination thereof.

11. The EPA-EE nano-lipid composition of claim 1, being in 1000 parts by mass and selected from the group consisting of following compositions:
[Composition 1-1]: 100 parts by mass of EPA-EE 60, 10 parts by mass of soybean phospholipid Lipoid S75, and a balance of water;
[Composition 1-2]: 100 parts by mass of EPA-EE 60, 10 parts by mass of soybean phospholipid Lipoid S100, and a balance of water;
[Composition 1-3]: 100 parts by mass of EPA-EE 60, 10 parts by mass of sunflower seed phospholipid Lipoid H100, and a balance of water;
[Composition 1-4]: 100 parts by mass of EPA-EE 60, 10 parts by mass of polyene phosphatidylcholine, and a balance of water;
[Composition 1-5]: 100 parts by mass of EPA-EE 60, 10 parts by mass of soybean phospholipid Lipoid S75, and a balance of water;
[Composition 1-6]: 200 parts by mass of EPA-EE 60, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and a balance of water;
[Compositions 1-7]: 200 parts by mass of EPA-EE 60, 50 parts by mass of soybean phospholipid Lipoid S100, 10 parts by mass of polysorbate 80, 0.5 parts by mass of α-tocopherol, 30 parts by mass of corn oil, and a balance of water;
[Compositions 1-8]: 200 parts by mass of EPA-EE 60, 50 parts by mass of sunflower seed phospholipid Lipoid H100, 10 parts by mass of polysorbate 80, 0.5 parts by mass of α-tocopherol, 30 parts by mass of corn oil, and a balance of water;
[Compositions 1-9]: 200 parts by mass of EPA-EE 60, 50 parts by mass of polyene phosphatidylcholine, 10 parts by mass of sorbitan oleate 80, 0.5 parts by mass of α-tocopherol, 30 parts by mass of olive oil, and a balance of water;
[Compositions 1-10]: 200 parts by mass of EPA-EE 60, 50 parts by mass of soybean phospholipid Lipoid S75, 10 parts by mass of sorbitan oleate 80, 0.5 parts by mass of α-tocopherol, 30 parts by mass of olive oil, and a balance of water;
[Compositions 1-11]: 300 parts by mass of EPA-EE 60, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and a balance of water;
[Compositions 1-12]: 300 parts by mass of EPA-EE 60, 100 parts by mass of soybean phospholipid Lipoid S100, 20 parts by mass of polysorbate 80, 1 part by mass of α-tocopherol, 50 parts by mass of corn oil, and a balance of water;
[Compositions 1-13]: 300 parts by mass of EPA-EE 60, 100 parts by mass of sunflower seed phospholipid Lipoid H100, 20 parts by mass of polysorbate 80, 1 part by mass of α-tocopherol, 50 parts by mass of corn oil, and a balance of water;
[Compositions 1-14]: 300 parts by mass of EPA-EE 60, 100 parts by mass of polyene phosphatidylcholine, 20 parts by mass of sorbitan oleate 80, 1 part by mass of α-tocopherol, 50 parts by mass of olive oil, and a balance of water;
[Compositions 1-15]: 300 parts by mass of EPA-EE 60, 100 parts by mass of soybean phospholipid Lipoid S75, 20 parts by mass of sorbitan oleate 80, 1 part by mass of α-tocopherol, 50 parts by mass of olive oil, and a balance of water;
[Composition 2-1]: 100 parts by mass of EPA-EE 80, 10 parts by mass of soybean phospholipid Lipoid S75, and a balance of water;
[Composition 2-2]: 100 parts by mass of EPA-EE 80, 10 parts by mass of soybean phospholipid Lipoid S100, and a balance of water;
[Composition 2-3]: 100 parts by mass of EPA-EE 80, 10 parts by mass of sunflower seed phospholipid Lipoid H100, and a balance of water;
[Composition 2-4]: 100 parts by mass of EPA-EE 80, 10 parts by mass of polyene phosphatidylcholine, and a balance of water;
[Composition 2-5]: 100 parts by mass of EPA-EE 80, 10 parts by mass of soybean phospholipid Lipoid S75, and a balance of water;
[Composition 2-6]: 200 parts by mass of EPA-EE 80, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and a balance of water;
[Compositions 2-7]: 200 parts by mass of EPA-EE 80, 50 parts by mass of soybean phospholipid Lipoid S100, 10 parts by mass of polysorbate 80, 0.5 parts by mass of α-tocopherol, 30 parts by mass of corn oil, and a balance of water;
[Compositions 2-8]: 200 parts by mass of EPA-EE 80, 50 parts by mass of sunflower seed phospholipid Lipoid H100, 10 parts by mass of polysorbate 80, 0.5 parts by mass of α-tocopherol, 30 parts by mass of corn oil, and a balance of water;
[Compositions 2-9]: 200 parts by mass of EPA-EE 80, 50 parts by mass of polyene phosphatidylcholine, 10 parts by mass of sorbitan oleate 80, 0.5 parts by mass of α-tocopherol, 30 parts by mass of olive oil, and a balance of water;
[Compositions 2-10]: 200 parts by mass of EPA-EE 80, 50 parts by mass of soybean phospholipid Lipoid S75, 10 parts by mass of sorbitan oleate 80, 0.5 parts by mass of α-tocopherol, 30 parts by mass of olive oil, and a balance of water;
[Compositions 2-11]: 300 parts by mass of EPA-EE 80, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and a balance of water;
[Compositions 2-12]: 300 parts by mass of EPA-EE 80, 100 parts by mass of soybean phospholipid Lipoid S100, 20 parts by mass of polysorbate 80, 1 part by mass of α-tocopherol, 50 parts by mass of corn oil, and a balance of water;
[Composition 2-13]: 300 parts by mass of EPA-EE 80, 100 parts by mass of sunflower seed phospholipid Lipoid H100, 20 parts by mass of polysorbate 80, 1 part by mass of α-tocopherol, 50 parts by mass of corn oil, and a balance of water;
[Composition 2-14]: 300 parts by mass of EPA-EE 80, 100 parts by mass of polyene phosphatidylcholine, 20 parts by mass of sorbitan oleate 80, 1 part by mass of α-tocopherol, 50 parts by mass of olive oil, and a balance of water;
[Composition 2-15]: 300 parts by mass of EPA-EE 80, 100 parts by mass of soybean phospholipid Lipoid S75, 20 parts by mass of sorbitan oleate 80, 1 part by mass of α-tocopherol, 50 parts by mass of olive oil, and a balance of water;
[Composition 3-1]: 100 parts by mass of EPA-EE 97, 10 parts by mass of soybean phospholipid Lipoid S75, and a balance of water;
[Composition 3-2]: 100 parts by mass of EPA-EE 97, 10 parts by mass of soybean phospholipid Lipoid S100, and a balance of water;
[Composition 3-3]: 100 parts by mass of EPA-EE 97, 10 parts by mass of sunflower seed phospholipid Lipoid H100, and a balance of water;
[Composition 3-4]: 100 parts by mass of EPA-EE 97, 10 parts by mass of polyene phosphatidylcholine, and a balance of water;
[Composition 3-5]: 100 parts by mass of EPA-EE 97, 10 parts by mass of soybean phospholipid Lipoid S75, and a balance of water;
[Composition 3-6]: 200 parts by mass of EPA-EE 97, 50 parts by mass of soybean phospholipid Lipoid S75, 10 parts by mass of egg yolk lecithin E80, 0.5 parts by mass of α-tocopherol, 30 parts by mass of a mixture of corn oil and olive oil in equal masses, and a balance of water;
[Compositions 3-7]: 200 parts by mass of EPA-EE 97, 50 parts by mass of soybean phospholipid Lipoid S100, 10 parts by mass of polysorbate 80, 0.5 parts by mass of α-tocopherol, 30 parts by mass of corn oil, and a balance of water;
[Compositions 3-8]: 200 parts by mass of EPA-EE 97, 50 parts by mass of sunflower seed phospholipid Lipoid H100, 10 parts by mass of polysorbate 80, 0.5 parts by mass of α-tocopherol, 30 parts by mass of corn oil, and a balance of water;
[Compositions 3-9]: 200 parts by mass of EPA-EE 97, 50 parts by mass of polyene phosphatidylcholine, 10 parts by mass of sorbitan oleate 80, 0.5 parts by mass of α-tocopherol, 30 parts by mass of olive oil, and a balance of water;
[Compositions 3-10]: 200 parts by mass of EPA-EE 97, 50 parts by mass of soybean phospholipid Lipoid S75, 10 parts by mass of sorbitan oleate 80, 0.5 parts by mass of α-tocopherol, 30 parts by mass of olive oil, and a balance of water;
[Compositions 3-11]: 300 parts by mass of EPA-EE 97, 100 parts by mass of soybean phospholipid Lipoid S75, 20 parts by mass of egg yolk lecithin E80, 1 part by mass of α-tocopherol, 50 parts by mass of a mixture of corn oil and olive oil in equal masses, and a balance of water;
[Composition 3-12]: 300 parts by mass of EPA-EE 97, 100 parts by mass of soybean phospholipid Lipoid S100, 20 parts by mass of polysorbate 80, 1 part by mass of α-tocopherol, 50 parts by mass of corn oil, and a balance of water;
[Composition 3-13]: 300 parts by mass of EPA-EE 97, 100 parts by mass of sunflower seed phospholipid Lipoid H100, 20 parts by mass of polysorbate 80, 1 part by mass of α-tocopherol, 50 parts by mass of corn oil, and a balance of water;
[Composition 3-14]: 300 parts by mass of EPA-EE 97, 100 parts by mass of polyene phosphatidylcholine, 20 parts by mass of sorbitan oleate 80, 1 part by mass of α-tocopherol, 50 parts by mass of olive oil, and a balance of water;
[Compositions 3-15]: 300 parts by mass of EPA-EE 97, 100 parts by mass of soybean phospholipid Lipoid S75, 20 parts by mass of sorbitan oleate 80, 1 part by mass of α-tocopherol, 50 parts by mass of olive oil, and a balance of water.

12. The EPA-EE nano-lipid composition of claim 1, comprising following components in parts by mass: 50 to 500 parts by mass of the EPA-EE raw material, 10 to 100 parts by mass of the first emulsifier, 0 to 60 parts by mass of the stabilizer, 0 to 50 parts by mass of the first auxiliary material, 20 to 40 parts by mass of the second auxiliary material, and water; and
further satisfying one or more of following features:
the total weight of the EPA-EE nano-lipid composition is 900 to 1100 parts by mass;
the EPA-EE raw material takes 100 to 400 parts by mass;
the first emulsifier takes 10 to 50 parts by mass;
the first emulsifier comprises soybean phospholipid;
the stabilizer takes 10 to 50 parts by mass;
the stabilizer comprises TPGS, DSPE-PEG, S40, or any combination thereof;
the first auxiliary material takes 10 to 30 parts by mass;
the first auxiliary material comprises phosphatidylserine, sodium glutamate, taurine, or any combination thereof;
the second auxiliary material is a combination of an antioxidant and a base oil.

13. The EPA-EE nano-lipid composition of claim 1, being in 1000 parts by mass and selected from the group consisting of following compositions in combination with the second auxiliary material, wherein the second auxiliary material is a combination of α-tocopherol in 1 part by mass and olive oil in 30 parts by mass, or is a combination of α-tocopherol in 2 parts by mass and a medium-chain triglyceride in 30 parts by mass:
[A1]: 100 parts by mass of EPA-EE 60, 10 parts by mass of soybean phospholipid Lipoid S75, 10 parts by mass of TPGS, and a balance of water;
[A2]: 100 parts by mass of EPA-EE 60, 10 parts by mass of DSPE-PEG, 10 parts by mass of sodium glutamate, and a balance of water;
[A3]: 100 parts by mass of EPA-EE 60, 10 parts by mass of S40, 10 parts by mass of phosphatidylserine, and a balance of water;
[A4]: 100 parts by mass of EPA-EE 60, 10 parts by mass of S40, 10 parts by mass of taurine, and a balance of water;
[A5]: 100 parts by mass of EPA-EE 60, 10 parts by mass of a mixture of TPGS and S40 in equal masses, 10 parts by mass of a mixture of taurine and sodium glutamate in equal masses, and a balance of water;
[A6]: 100 parts by mass of EPA-EE 60, 10 parts by mass of soybean phospholipid Lipoid S75, 10 parts by mass of TPGS, 10 parts by mass of phosphatidylserine, and a balance of water;
[A7]: 100 parts by mass of EPA-EE 60, 10 parts by mass of soybean phospholipid Lipoid S75, 10 parts by mass of DSPE-PEG, 10 parts by mass of sodium glutamate, and a balance of water;
[A8]: 100 parts by mass of EPA-EE 60, 10 parts by mass of soybean phospholipid Lipoid S75, 10 parts by mass of S40, 10 parts by mass of phosphatidylserine, and a balance of water;
[A9]: 100 parts by mass of EPA-EE 60, 10 parts by mass of soybean phospholipid Lipoid S75, 10 parts by mass of S40, 10 parts by mass of taurine, and a balance of water;
[A10]: 100 parts by mass of EPA-EE 60, 10 parts by mass of soybean phospholipid Lipoid S75, 10 parts by mass of a mixture of TPGS and S40 in equal masses, 10 parts by mass of a mixture of taurine and sodium glutamate in equal masses, and a balance of water;
[A11]: 200 parts by mass of EPA-EE 60, 100 parts by mass of soybean phospholipid Lipoid S75, 20 parts by mass of TPGS, 20 parts by mass of phosphatidylserine, and a balance of water;
[A12]: 200 parts by mass of EPA-EE 60, 100 parts by mass of soybean phospholipid Lipoid S75, 20 parts by mass of DSPE-PEG, 20 parts by mass of sodium glutamate, and a balance of water;
[A13]: 200 parts by mass of EPA-EE 60, 100 parts by mass of soybean phospholipid Lipoid S75, 20 parts by mass of S40, 20 parts by mass of phosphatidylserine, and a balance of water;
[A14]: 200 parts by mass of EPA-EE 60, 100 parts by mass of soybean phospholipid Lipoid S75, 20 parts by mass of S40, 20 parts by mass of taurine, and a balance of water;
[A15]200 parts by mass of EPA-EE 60, 100 parts by mass of soybean phospholipid Lipoid S75, 20 parts by mass of a mixture of TPGS and S40 in equal masses, 20 parts by mass of a mixture of taurine and sodium glutamate in equal masses, and a balance of water;
[B1]: 100 parts by mass of EPA-EE 80, 10 parts by mass of soybean phospholipid Lipoid S75, 10 parts by mass of TPGS, and a balance of water;
[B2]: 100 parts by mass of EPA-EE 80, 10 parts by mass of DSPE-PEG, 10 parts by mass of sodium glutamate, and a balance of water;
[B3]: 100 parts by mass of EPA-EE 80, 10 parts by mass of S40, 10 parts by mass of phosphatidylserine, and a balance of water;
[B4]: 100 parts by mass of EPA-EE 80, 10 parts by mass of S40, 10 parts by mass of taurine, and a balance of water;
[B5]: 100 parts by mass of EPA-EE 80, 10 parts by mass of a mixture of TPGS and S40 in equal masses, 10 parts by mass of a mixture of taurine and sodium glutamate in equal masses, and a balance of water;
[B6]: 100 parts by mass of EPA-EE 80, 10 parts by mass of soybean phospholipid Lipoid S75, 10 parts by mass of TPGS, 10 parts by mass of phosphatidylserine, and a balance of water;
[B7]: 100 parts by mass of EPA-EE 80, 10 parts by mass of soybean phospholipid Lipoid S75, 10 parts by mass of DSPE-PEG, 10 parts by mass of sodium glutamate, and a balance of water;
[B8]: 100 parts by mass of EPA-EE 80, 10 parts by mass of soybean phospholipid Lipoid S75, 10 parts by mass of S40, 10 parts by mass of phosphatidylserine, and a balance of water;
[B9]: 100 parts by mass of EPA-EE 80, 10 parts by mass of soybean phospholipid Lipoid S75, 10 parts by mass of S40, 10 parts by mass of taurine, and a balance of water;
[B10]: 100 parts by mass of EPA-EE 80, 10 parts by mass of soybean phospholipid Lipoid S75, 10 parts by mass of a mixture of TPGS and S40 in equal masses, 10 parts by mass of a mixture of taurine and sodium glutamate in equal masses, and a balance of water;
[B11]: 200 parts by mass of EPA-EE 80, 100 parts by mass of soybean phospholipid Lipoid S75, 20 parts by mass of TPGS, 20 parts by mass of phosphatidylserine, and a balance of water;
[B12]: 200 parts by mass of EPA-EE 80, 100 parts by mass of soybean phospholipid Lipoid S75, 20 parts by mass of DSPE-PEG, 20 parts by mass of sodium glutamate, and a balance of water;
[B13]: 200 parts by mass of EPA-EE 80, 100 parts by mass of soybean phospholipid Lipoid S75, 20 parts by mass of S40, 20 parts by mass of phosphatidylserine, and a balance of water;
[B14]: 200 parts by mass of EPA-EE 80, 100 parts by mass of soybean phospholipid Lipoid S75, 20 parts by mass of S40, 20 parts by mass of taurine, and a balance of water;
[B15]: 200 parts by mass of EPA-EE 80, 100 parts by mass of soybean phospholipid Lipoid S75, 20 parts by mass of a mixture of TPGS and S40 in equal masses, 20 parts by mass of a mixture of taurine and sodium glutamate in equal masses, and a balance of water;
[C1]: 100 parts by mass of EPA-EE 97, 10 parts by mass of soybean phospholipid Lipoid S75, 10 parts by mass of TPGS, and a balance of water;
[C2]: 100 parts by mass of EPA-EE 97, 10 parts by mass of DSPE-PEG, 10 parts by mass of sodium glutamate, and a balance of water;
[C3]: 100 parts by mass of EPA-EE 97, 10 parts by mass of S40, 10 parts by mass of phosphatidylserine, and a balance of water;
[C4]: 100 parts by mass of EPA-EE 97, 10 parts by mass of S40, 10 parts by mass of taurine, and a balance of water;
[C5]: 100 parts by mass of EPA-EE 97, 10 parts by mass of a mixture of TPGS and S40 in equal masses, 10 parts by mass of a mixture of taurine and sodium glutamate in equal masses, and a balance of water;
[C6]: 100 parts by mass of EPA-EE 97, 10 parts by mass of soybean phospholipid Lipoid S75, 10 parts by mass of TPGS, 10 parts by mass of phosphatidylserine, and a balance of water;
[C7]: 100 parts by mass of EPA-EE 97, 10 parts by mass of soybean phospholipid Lipoid S75, 10 parts by mass of DSPE-PEG, 10 parts by mass of sodium glutamate, and a balance of water;
[C8]: 100 parts by mass of EPA-EE 97, 10 parts by mass of soybean phospholipid Lipoid S75, 10 parts by mass of S40, 10 parts by mass of phosphatidylserine, and a balance of water;
[C9]: 100 parts by mass of EPA-EE 97, 10 parts by mass of soybean phospholipid Lipoid S75, 10 parts by mass of S40, 10 parts by mass of taurine, and a balance of water;
[C10]: 100 parts by mass of EPA-EE 97, 10 parts by mass of soybean phospholipid Lipoid S75, 10 parts by mass of a mixture of TPGS and S40 in equal masses, 10 parts by mass of a mixture of taurine and sodium glutamate in equal masses, and a balance of water;
[C11]: 200 parts by mass of EPA-EE 97, 100 parts by mass of soybean phospholipid Lipoid S75, 20 parts by mass of TPGS, 20 parts by mass of phosphatidylserine, and a balance of water;
[C12]: 200 parts by mass of EPA-EE 97, 100 parts by mass of soybean phospholipid Lipoid S75, 20 parts by mass of DSPE-PEG, 20 parts by mass of sodium glutamate, and a balance of water;
[C13]: 200 parts by mass of EPA-EE 97, 100 parts by mass of soybean phospholipid Lipoid S75, 20 parts by mass of S40, 20 parts by mass of phosphatidylserine, and a balance of water;
[C14]: 200 parts by mass of EPA-EE 97, 100 parts by mass of soybean phospholipid Lipoid S75, 20 parts by mass of S40, 20 parts by mass of taurine, and a balance of water;
[C15]: 200 parts by mass of EPA-EE 97, 100 parts by mass of soybean phospholipid Lipoid S75, 20 parts by mass of a mixture of TPGS and S40 in equal masses, 20 parts by mass of a mixture of taurine and sodium glutamate in equal masses, and a balance of water.

14. An EPA-EE nano-lipid formulation, comprising the EPA-EE nano-lipid composition of om claim 1.

15. The EPA-EE nano-lipid formulation of claim 14, being an oral formulation.

16. A method for preparing the EPA-EE nano-lipid formulation of claim 14, comprising following steps:
mixing oil phase components comprising the EPA-EE raw material under a heating condition to prepare an oil phase matrix;
dissolving aqueous phase components into an aqueous solvent to prepare an aqueous phase matrix, or adopting water as the aqueous phase matrix;
mixing and shear-stirring the oil phase matrix and the aqueous phase matrix to prepare an oil-in-water primary emulsion; and
subjecting the oil-in-water primary emulsion to high pressure homogenization to prepare a submicron emulsion.

17. (canceled)

18. A method for preventing or treating a cardiovascular disease, comprising administering to a subject a therapeutically effective amount of the EPA-EE nano-lipid composition of claim 1.

19. The method of claim 18, satisfying any one or any combination of following features:
the cardiovascular disease is one or more events selected from the group consisting of hyperlipidemia, severe hypertriglyceridemia, extreme hypertriglyceridemia, atherosclerosis, occlusive atherosclerosis accompanied by ulceration and/or pain and coldness, carotid artery plaque, myocardial infarction, ischemic heart attack, ischemic attack, acute angina pectoris, hospitalization for acute angina pectoris, stroke, and hospitalization for a cardiovascular event;
the administering is by oral;
the subject is a patient suffering from the cardiovascular disease;
the subject is a mammal.

20-21. (canceled)

22. The EPA-EE nano-lipid composition of claim 1, wherein based on the total weight of the EPA-EE nano-lipid composition, a content of the stabilizer is in a range from 0.01% to 5%;
the stabilizer is an amphiphilic non-ionic polymer, and the stabilizer is selected from the group consisting of a vitamin lipid polymer derivative, a phospholipid polymer derivative, a fatty acid ester polymer derivative, and a polyoxyethylene-polyoxypropylene ether block copolymer, and any combination thereof,
the vitamin lipid polymer derivative is a vitamin E polyethylene glycol succinate;
the phospholipid polymer derivative is a polyethylene glycol-modified synthetic phospholipid;
the fatty acid ester polymer derivative is a polyethylene glycol-modified fatty acid ester;
a molecular weight of a PEG unit in the phospholipid polymer derivative is in a range from 400 Da to 6000 Da;
a molecular weight of an PEG unit in the fatty acid ester polymer derivative is in a range from 200 Da to 4000 Da;
the molecular weight refers to a number-average molecular weight or a weight-average molecular weight.

23. The EPA-EE nano-lipid composition of claim 1, wherein based on the total weight of the EPA-EE nano-lipid composition, a content of the first auxiliary material is in a range from 0.01% to 5% (w/w);
the first auxiliary material is selected from the group consisting of an amino acid with a side chain having a negatively charged group, an amino acid derivative with a negatively charged group, and a small peptide with a side chain having a negatively charged group, and any combination thereof,
the amino acid with the side chain having the negatively charged group is selected from the group consisting of aspartic acid, glutamic acid, taurine, and any combination thereof;
the amino acid derivative with the side chain having the negatively charged group is selected from the group consisting of phosphatidylserine, dihexadecyl-glutamate-glutamine, dihexadecyl-glutamate-glutamic acid, dihexadecyl-glutamate-asparagine, and any combination thereof;
the small peptide with the side chain having the negatively charged group is glutathione.
